(19) United States
(12) Patent Application Publication
(10) Pub. No.: US 2020/0102281 A1
(43) Pub. Date: Apr. 2, 2020

(54) FREE RADICAL- AND REACTIVE OXYGEN SPECIES-REACTING COMPOUNDS

(71) Applicant: Christopher Duke, San Diego, CA (US)

(72) Inventor: Christopher Duke, San Diego, CA (US)

(21) Appl. No.: 16/467,965

(22) PCT Filed: Nov. 29, 2017
(86) PCT No.: PCT/US17/63800

§ 371 Date: Jun. 8, 2019

Related U.S. Application Data
(60) Provisional application No. 62/428,137, filed on Nov. 30, 2016.
(60) Provisional application No. 62/542,404, filed on Aug. 8, 2017.
(60) Provisional application No. 62/558,520, filed on Sep. 14, 2017.
(60) Provisional application No. 62/566,706, filed on Oct. 2, 2017.

(51) Int. Cl. C07D 311/72; C07C 29/48; C07C 403/14; C07C 45/40; C07C 67/05; C08L 29/10; C08L 33/14

(52) U.S. Cl. CPC C07D 311/72; C07C 29/48; C07C 403/14; A61K 45/06; C07C 67/05; C08L 29/10; C08L 33/14; C07C 45/40

(57) ABSTRACT

Provided are compounds that generate a peroxide when they react with ozone in the presence of water. Additionally, alkyne compounds reactive with a free radical, a reactive oxygen species (ROS) or another reactive species are provided. Also provided are enol ether, enamine, and vinal thioester compounds reactive with a free radical, a strong acid, a reactive oxygen species (ROS) or another reactive species. Additionally provided are compounds reactive with a free radical, an ROS or another reactive species. The compounds comprise a conjugated moiety operably joined to an alkene moiety and a resonance-transmitting moiety, wherein the resonance-transmitting moiety transmits an electron through the conjugated moiety to the alkene moiety, which reacts with the free radical, an ROS or another reactive species. Also provided are methods of decomposing a free radical, an ROS or another reactive species. The methods comprise contacting the free radical or ROS with any of the above compounds. Also provided are methods of using any of the compounds described herein, and compositions comprising those compounds. Additionally provided are methods of producing the above compounds.

### CROSS-REFERENCE TO RELATED APPLICATIONS

This application claims the benefit of U.S. Provisional Application No. 62/428,137, filed Nov. 30, 2016; U.S. Provisional Application No. 62/542,404, filed Aug. 8, 2017; U.S. Provisional Application No. 62/558,520, filed Sep. 14, 2017; and U.S. Provisional Application No. 62/566,706, filed Oct. 2, 2017, all of which are incorporated by reference herein in their entirety.

### BACKGROUND OF THE INVENTION

### (1) Field of the Invention

The present application generally relates to compounds that react with free radicals and reactive oxygen species (ROS). More specifically, the application is directed to compounds having an enol ether, an enamine, a vinyl thioether moiety and/or conjugated moieties that react with, and decompose or degrade free radicals, reactive oxygen species, and other reactive species, including compounds that generate peroxides by ozonolysis, and methods and compositions that employ those compounds.

### (2) Description of the Related Art

Compounds having free radicals, reactive oxygen species and other reactive chemical species (e.g., reactive nitrogen species, reactive sulfur species, and reactive phosphorous species) are common in the environment and in biological systems, where they contribute to multiple diseases and deleterious conditions. See, e.g., Arulselvan et al., 2016; Panth et al., 2016; Ullah et al., 2015; Lalkovicova and Danielisova, 2016; Finosh et al., 2013; Braverman and Moser, 2012.

Non-limiting examples of free radicals are superoxide, hydrogen peroxide, hydroxyl radical, nitric oxide, peroxynitrite, hypochlorous acid, organic radicals, peroxy radical, alkoxy radical, thiyl radical, sulfonyl radical, and thiyl peroxyl radical; Nonlimiting examples of ROS are singlet oxygen, dioxygen, triplet oxygen, ozone (including atmospheric ozone), nitrogen oxides, ozonide, dioxygenyl cation, atomic oxygen, sulfur oxides, ammonia, carbon monoxide, peroxides including (but not limited to) hydrogen peroxide, organic hydroperoxides, peroxide ion, organic peroxides, peracids, peroxysulfuric acid, peroxymonosulfur acid, peroxydisulfuric acid, peroxyphosphoric acid, meta-chloroperoxybenzoic acid, peresters, peracetic acid, performic acid, and nitrosoperoxycarbonate anion; nitrocarbonate anion, dinitrogen dioxide, nitronium, atomic oxygen, and hydroxyl anion.

Ozone (O₃) is a triatomic molecule composed of three oxygen atoms. It is formed from diatomic oxygen (O₂) by the action of sunlight, ultraviolet light or an electrical discharge. Scheme 1 illustrates the resonance structures of ozone.

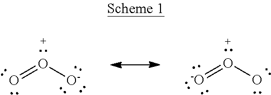

Ozone is formed in the atmosphere by the action of sunlight, ultraviolet light or an electrical discharge such as lightning on oxygen in the air. Nitrogen oxide (NOx) air pollutants in the atmosphere also react with volatile organic compounds in the air to form ozone in sunlight. It is also formed when an electrical apparatus produces sparks in the air. The generated ozone can react with other things, such as plants or rubber to produce more volatile organic compounds. These volatile compounds are released into the air and ultimately produce more ozone.

Ozone reacts with alkenes and alkynes to form organic compounds in a process known as ozonolysis. The multiple bonds in these compounds are oxidized by the action of ozone to provide compounds in which the double bonds form a carbonyl group. The outcome of the reaction depends on the type of multiple bonds being oxidized. For example, alkenes can be oxidized by ozone to form aldehydes, ketones, carboxylic acids, esters, amides, enones, acyl halides, imides, acid anhydrides, 1,3-dicarbonyls, carbamates, carbazides, carbazones, carboxylates, cyclic imides, formates, furazones, hydrazines, hydroxamates, isocyanates, lactams, lactones, semicarbazones, ureas, thioesters, thiocarbamates, dithiocarbamates, etc. Often, two aldehydes and/or ketones are produced when the olefinic compound is appropriately substituted. Scheme 2 illustrates an ozonolysis reaction between a carbon-carbon double bond and ozone. The reaction provides two carbonyl containing compounds depending upon the R substituents.

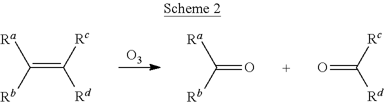

Ozone in the air may be toxic to human beings and animals. According to Occupational Safety and Health Administration (OSHA), the permissible maximal average concentration of ozone in the air should be no more than 0.1 ppm when breathing air. Many apparatuses for industrial use are manufactured in accordance with these standards. Ozone has a characteristic odor, which is noticeable even at concentrations as low as 0.01 to 0.02 ppm. When the concentration of ozone increases to about 0.05 ppm, it has an unpleasant odor; and when the concentration exceeds 0.1 ppm, it is irritating to the mucous membranes of the eyes and respiratory organs. Ozone is also a powerful oxidizing agent which oxidizes and deteriorates organic materials. Therefore, it is desirable that the concentration of ozone be kept as low as possible.

Ozone is used in industry for the sterilization, deodorization and decolorization of water and for the treatment of raw sewage. These applications often require the use of ozone in concentrations as high as 500-2500 ppm. For example, to sterilize water, 1 to 3 g of ozone is bubbled into 1 cubic meter of water. Most of the ozone blown into water is decomposed, however, some of the residual ozone can be discharged from the water into the air. Since the concentration of the discharged ozone in the air may be as high as 1 ppm, it is necessary to decompose the discharged ozone before it spreads into the air for the safety to human beings and for the protection of the environment.

Since ozone is toxic to human beings when its concentration in the air is high, various methods have been proposed to decrease its concentration. For example, filters made of activated carbon and filters containing various catalysts, such as metal oxides of manganese, copper, silver and cobalt, have been employed for decomposing ozone. If the density of the materials in these filters is high, the absorption of ozone and its decomposition efficiency is increased. However, the higher density of these materials slows the flow rate of the air through the filter. By contrast, if the density of the materials in the filter is decreased, the absorption of ozone and the ozone decomposition efficiency are decreased.

Various polymers and terpenoid compounds have also been used to control ozone levels. For example, a rubber olefin polymer containing double bond groups has been used for decomposing ozone generated from an electrophotographic copying machine. Terpenoid compounds capable of decomposing ozone, such as linalool, linalool ester, citral and the like, in various solutions and gels have also been used. In addition, paints containing a variety of organic materials have been proposed. However, the decomposition efficiency is not high enough for use in practice. Furthermore, the by-products formed after decomposition of the ozone has not been fully characterized in these cases. Therefore, it is unclear whether exposure to these by-products affect a person's health, and whether there are any negative environmental impacts.

Therefore, there remains a need in the art for new compounds, compositions and methods for removing and/or controlling ozone levels without having a negative impact on humans, animals and the environment, wherein the by-products formed after decomposition of the ozone is safe and fully characterized.

Ozone decomposing compounds are provided in PCT/US2015/044388, published as WO 2016/023015. In particular, ozone degrading polymers and small molecules are provided therein that, upon reaction with ozone, are converted into non-toxic compounds, e.g., sugars, benzaldehyde, citral, vanillin, raspberry ketone and camphor. Those compounds can be incorporated into various products, such as paints, air-filters, clothing, water-filters, agriculture, crop care and food care products, beauty products, surface and coating products, cleaning products, air care products, personal care products, home care products, flavor components, preservatives, antioxidants, cosmetics, lotions and shampoos.

Additional ozone degrading compounds are provided in PCT/US2016/052529, published as WO 2017/049305. That application provides additional small molecules, including inactive molecules that become active, useful compounds upon reaction with ozone. Examples of those active compounds are pharmaceuticals (where the inactive molecule serves as a prodrug activated by ozone), dyes, specific binding agents, biocides, clothing, air-filters, fertilizers, water-filters, agriculture, crop care and food care products, beauty products, surface and coating products, cleaning products, air care products, personal care products, home care products, cosmetics, lotions, shampoos, disinfectants, antioxidants, preservatives, and flavor components.

The present invention provides compounds having a free radical-, ROS (including ozone), and other reactive species-reactive double and triple bond that generate peroxides, have resonance structures that promote reaction with the free radical and reactive species.

### BRIEF SUMMARY OF THE INVENTION

The present invention is directed to alkenes and alkynes that react with free radicals, reactive oxygen species and other reactive species.

In some embodiments, compounds that generate a peroxide when they react with ozone in the presence of water are provided. The compounds comprise the structure I

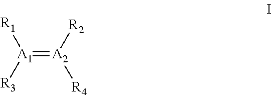

wherein A1 and A2 are independently C, O, N, S, Si or P and R1, R2, R3 and R4 are each a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl. Methods of generating a peroxide using these compounds is also provided.

Additionally, compounds reactive with a free radical, a reactive oxygen species (ROS) or another reactive species are provided, the compound comprises the structure LV, LVI or LVII

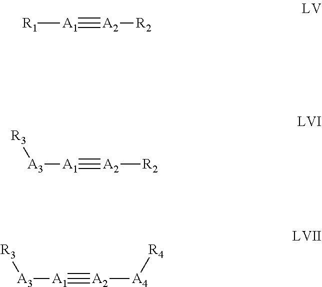

wherein A1, A2, A3, and A4 is each independently C, O, S, N or P and R₁, R2, R3, and R4 is each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

Also provided are compounds reactive with a free radical, a strong acid, a reactive oxygen species (ROS) or another reactive species. The compounds comprise the structure LXXXXII, LXXXIII, LXXXIV, LXXXV, or LXXXVI

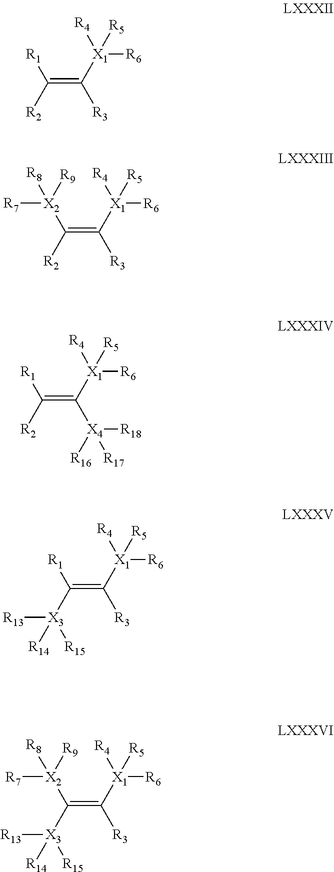

wherein

X₁, X₂, X₃ and X₄ are each independently O, N, P or S; and

R₁-R₁₈ are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl, provided R⁴ is not H or a lone pair of electrons.

Additionally provided are compounds reactive with a free radical, an ROS or another reactive species. The compounds comprise a conjugated moiety operably joined to an alkene moiety and a resonance-transmitting moiety, wherein the resonance-transmitting moiety transmits an electron through the conjugated moiety to the alkene moiety, which reacts with the free radical, an ROS or another reactive species.

Further provided are additional compounds reactive with a free radical, an ROS or another reactive species. The compounds comprise a conjugated moiety operably joined to an alkene moiety and, optionally, a resonance-transmitting moiety. In these compounds, the conjugated moiety or the resonance-transmitting moiety can be stimulated by an external energy source to excite an electron and/or transmit an electron through the conjugated moiety to the alkene moiety, which reacts with the free radical, strong acid, RNS or ROS.

Also provided are methods of decomposing a free radical, an ROS or another reactive species. The methods comprise contacting the free radical or ROS with any of the above compounds.

Additionally provided are methods of using any of the compounds described herein, and compositions comprising those compounds.

Further provided is a method of producing the compound CXLVIII

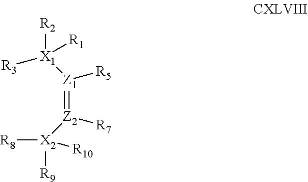

wherein

CXLVIII can be cis or trans, or a mixture thereof;

X₁ and X₂ are each independently O, P, N, C, Si or S;

Z₁ and Z₂ are each independently C, S, P, N or Si; and

R₁, R₂, R₃, R₄, R₅, R₆, R₇, R₅, R₉, and R₁₀ are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl,

the method comprising

combine compound CXLIX with CL in the presence of a first deprotonating agent or a base

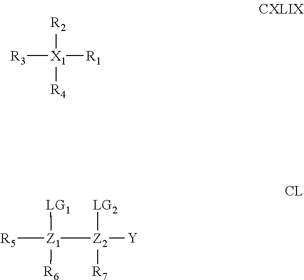

wherein

Y is a halogen, and

LG₁ and LG₂ are each independently a leaving group selected from the group consisting of a halogen, a tosylate, a mesylate, water, an alcohol, dinitrogen, a dialkyl ether, a perfluoroalkylsulfonate, a nitrate, a phosphate, an inorganic ester, an ester, a thioether, an amine, ammonia, a carboxylate, an aromatic, a substituted amine, an amide, an alkoxide, and a hydroxide,

to form Mixture 1;

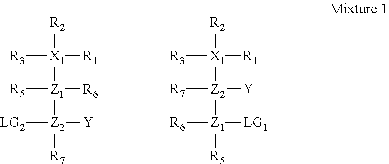

combine Mixture 1 with compound CLI in the presence of a second deprotonating agent or base to form Mixture 2;

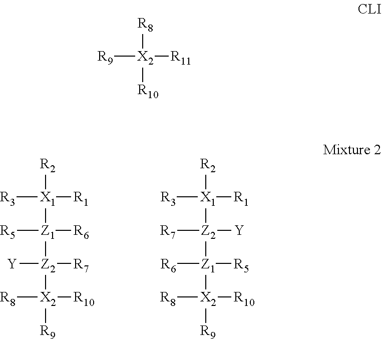

wherein R₁₁ is a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl,

and react Mixture 2 with a third deprotonating agent or base to form CXLVIII.

### BRIEF DESCRIPTION OF THE SEVERAL VIEWS OF THE DRAWINGS

FIG. 1 is an illustration of reactions that can occur with an exemplary invention chemotherapeutic prodrug.

FIG. 2 is an illustration that can occur with an exemplary invention antiviral prodrug.

FIG. 3 is an illustration of reactions that can occur with an exemplary invention composition that can serve as a food or skin care product.

FIG. 4 shows a generalized synthesis method for invention compounds.

FIG. 5 shows an embodiment of a synthesis method for invention compounds.

FIG. 6 shows an embodiment of a synthesis method for invention compounds.

### DETAILED DESCRIPTION OF THE INVENTION

As used herein, the singular forms “a”, “an” and “the” are intended to include the plural forms as well, unless the context clearly indicates otherwise. Additionally, the use of “or” is intended to include “and/or”, unless the context clearly indicates otherwise.

As used herein, “a free radical, an ROS or another reactive species” includes any of the following: organic peroxides, peracids, dioxygenyls, hypochlorite, reactive halogenated compounds, peroxy salts, alkoxides, reactive phosphorous oxides, peroxynitrite, nitric acid, sulfuric acid, phosphoric acid, nitrosoperoxycarbonate, carbonate radical, dinitrogen trioxide, nitrogen dioxide, hydroxyl ion, nitrous oxide, peroxynitrate, peroxynitrous acid, nitroxyl anion, nitrous acid, nitryl chloride, nitrosyl cation, hypochloric acid, hydrochloric acid, lipid peroxyl, peroxyl, peroxynitrite, alkyl peroxides, alkyl peroxynitrites, perhydroxyl radicals, diatomic oxygen, free electrons, sulfur dioxide, free radicals, superoxide, hydrogen peroxide, hydroxyl radical, nitric oxide, peroxynitrite, hypochlorous acid, persulfides, polysulfides, thiosulfates, organic radicals, peroxy radical, alkoxy radical, thiyl radical, sulfonyl radical, thiyl peroxyl radical, sulfur polycations, sulfides, oxoacids, oxoanions, sulfur trioxide, sulfites, pyrosulfuric acid, sodium dithionite, dithionite, oxyhalides, sulfuric acid derivatives, hydrogen sulfide, sulfurous acid, reactive oxygen species, reactive nitrogen species, reactive sulfur species, reactive phosphorous species, singlet oxygen, dioxygen, triplet oxygen, ozone (including atmospheric ozone), reactive nitrogen oxides, reactive sulfur oxides, ozonide, dioxygenyl cation, atomic oxygen, carbon monoxide, peroxides, organic hydroperoxides, nitrosoperoxycarbonate anion, nitrocarbonate anion, dinitrogen dioxide, nitronium, atomic oxygen, hydroxyl anion, or chlorine.

In all compounds provided herein, where relevant and not otherwise specified, X, Y, Z or n is an integer from 1 to 1,000,000 and R₁ is a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group (e.g., O, N, P, or S), conjugated or unconjugated groups, electron donating and/or electron withdrawing conjugated and/or unconjugated groups, a halogen, substituted or unsubstituted arylalkyl, or substituted or unsubstituted heteroarylalkyl. Where more than one R groups are present, two R groups can join together to form a ring structure.

### Generation of Peroxides

When any of the alkene compounds provided herein, and any of the compounds described in PCT Publications WO 2016/023015 and WO 2017/049305, are exposed to ozone in the presence of water, a peroxide is formed. Depending on the structure, the peroxide formed is hydrogen peroxide, an organic peroxide, an organic hydroperoxide, a peracid, a peroxide ion, superoxide, benzoyl peroxide, performic acid, peracetic acid, meta-chloroperoxybenzoid acid, peroxybenzoic acid, a peroxy acid, or R—O—O—R. Owing to their high reactivity, generally resulting in nontoxic or beneficial products (e.g., oxygen, carbon dioxide, water, acetate, glycolic acid, carboxylic acids, carboxylates, etc.), these products are very useful for, e.g., skin care and cleaning products, where the compound protects against skin damage by ozone, and the peroxide provides an antibacterial effect.

Without being bound by a particular mechanism, ozone reacts with unsaturated hydrocarbons with the cycloaddition of ozone to the C═C double bond to form a primary cyclic ozonide with a C—C single bond. Because of the large exothermicity of this reaction, rapid cleavage of this C—C bond and an O—O bond of the ozonide occurs to form a carbonyl molecule and a carbonyl oxide, commonly referred to as the Criegee intermediate. See, e.g., Wynalda and Murphy, 2010; Kalinowsky et al., 2014; Womack et al., 2015; Taatjes, 2017; Su et al., 2014; Nguyen et al., 2016; Liu et al., 2014; Hasson et al., 2001; Long et al., 2016.

In the presence of water (including liquid or gaseous water, e.g., atomospheric water vapor), the reaction follows one of the routes shown in Schemes 3 and 4 below:

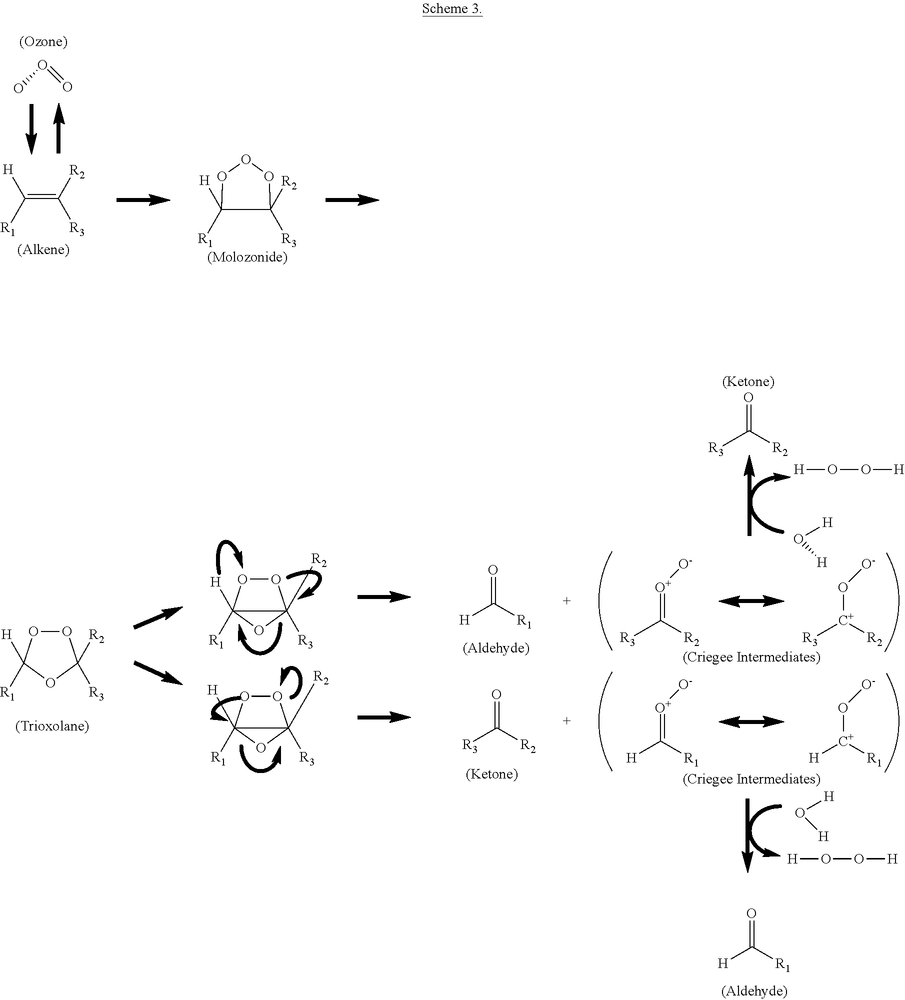

In Scheme 3, aldehydes and ketones are the end by-products from these reactions, but Criegee intermediates are also formed. Those intermediates two different resonance structures as shown apart, forming two products, two oxygen atoms from the ozone molecule go to one side while one oxygen goes to the other side. The structure above shows two pathways from the trioxolane group, where the electrons can transfer in two different ways. The Criegee intermediate reacts with a water molecule to form a hydrogen peroxide, or reacts with another molecule, which can form another type of peroxide, depending on the starting molecule. There are two ways to form the Criegee intermediates, and the aldehydes, and ketones. One mechanism is shown in Scheme 3, where the alkene forms a molozonide, and then a trioxolane, which then breaks apart into the aldehydes and ketones. The other mechanism is shown in Scheme 4 below, which skips the trioxolane.

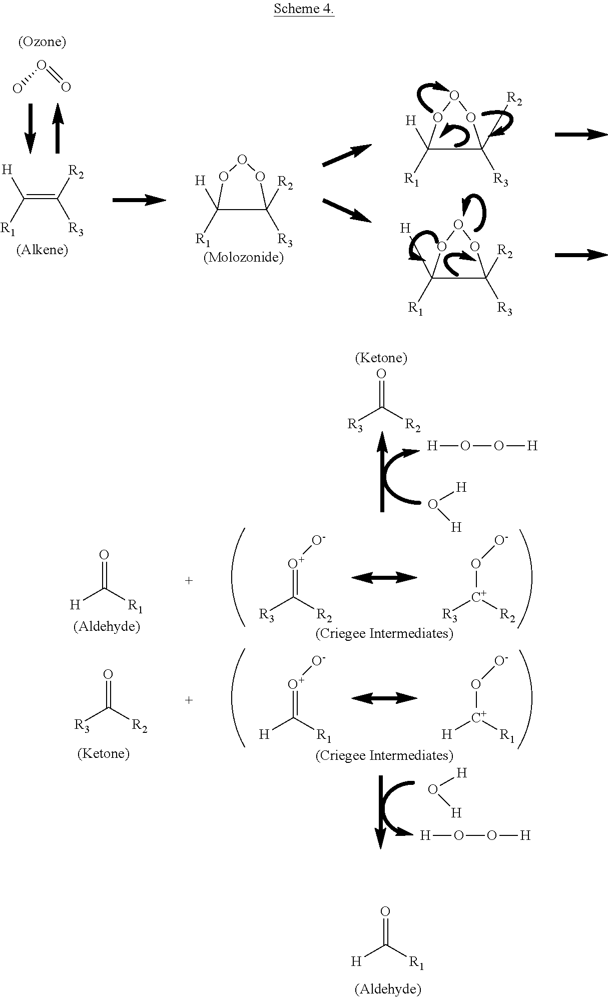

Criegee intermediates are more unstable than aldehydes and ketones, and have a much larger partial positive charge on their carbonyl carbon. Even when there is a double bond between the oxygen and carbonyl carbon in the Criegee intermediate, there is a positive charge on the oxonium oxygen which causes a large pull of electron density from the carbonyl carbon. This is result of the Criegee intermediate chemistry, and having an extra oxygen attached onto the carbonyl group. The result is a reactive Criegee intermediate that can act as both an electrophile and nucleophile, by either wanting to react with an electrophile, such as with a hydrogen on a water molecule, and with the partially positive charged carbonyl carbon wanting to react with a nucleophile, such as the oxygen atom in a water molecule.

To expand on Criegee intermediates and Scheme 3, the chemistry it has is very different than a traditional aldehyde or ketone, as a result of the more electrophilic properties of the carbonyl carbon, the nucleophilic properties of the end oxygen atom on the intermediate, and the resonance mechanisms that occurs on it. This allows the oxygen atom to potentially react with an electrophile, to form hydrogen peroxide, or another peroxide, while forming the aldehyde and ketone end byproduct molecule.

The high degree of positive charge present on a carbonyl carbon also allows it to be the target of a nucleophilic attack by a molecule, or a water molecule. When this attack occurs on an aldehyde or ketone, which should occur less than a Criegee intermediate because there is not as much of a partial positive charge on the carbonyl carbon of a ketone or aldehyde versus a Criegee intermediate, the water molecule will just simply fall off again, so the aldehyde or ketone will stay the same (see Scheme 5 below).

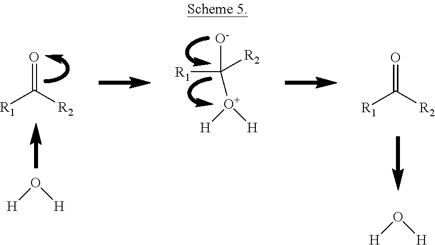

The chemistry is different with an oxygen, sulfur, nitrogen or phosphine on one of the carbons in the alkene. For example, Scheme 6 shows a Criegee intermediate during the reaction of ozone with an enol ether.

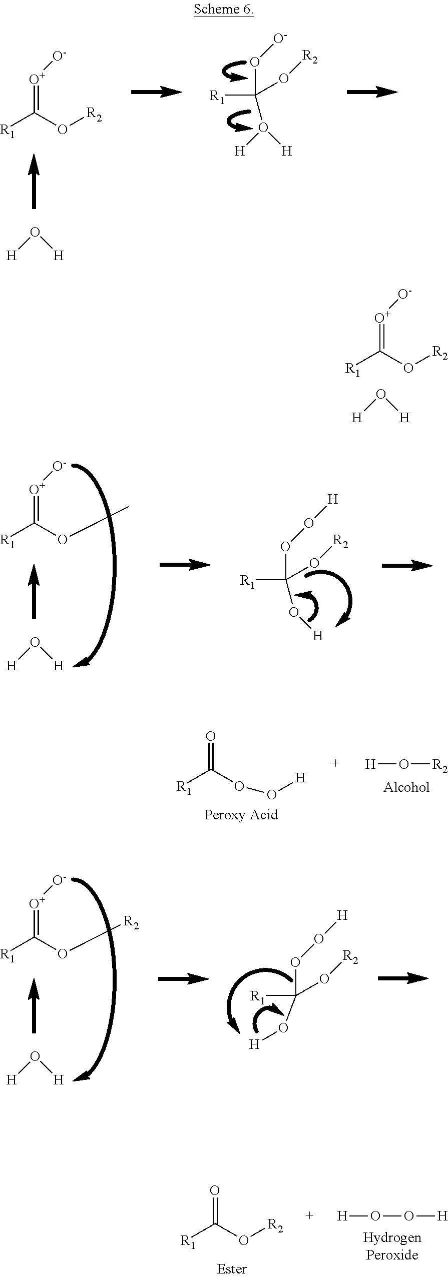

When an oxygen atom on a water molecule attacks the carbonyl carbon of the Criegee intermediate, the water molecule can either fall off again, or cause another alcohol group to fall off, such as the oxygen atom contained in the enol ether group. What happens in this case is the formation of a peroxy acid, in this case performic acid. This is demonstrated in Wynalda and Murphy, 2010, showing that reaction of ozone with plasmalogen glycerophosphoethanolamine lipids leads to the formation of performic acid. Performic acid is non-toxic, as a result of its safe byproducts upon degradation.

In light of the above, the mechanism of ozonolysis of an enol ether is provided as Scheme 7.

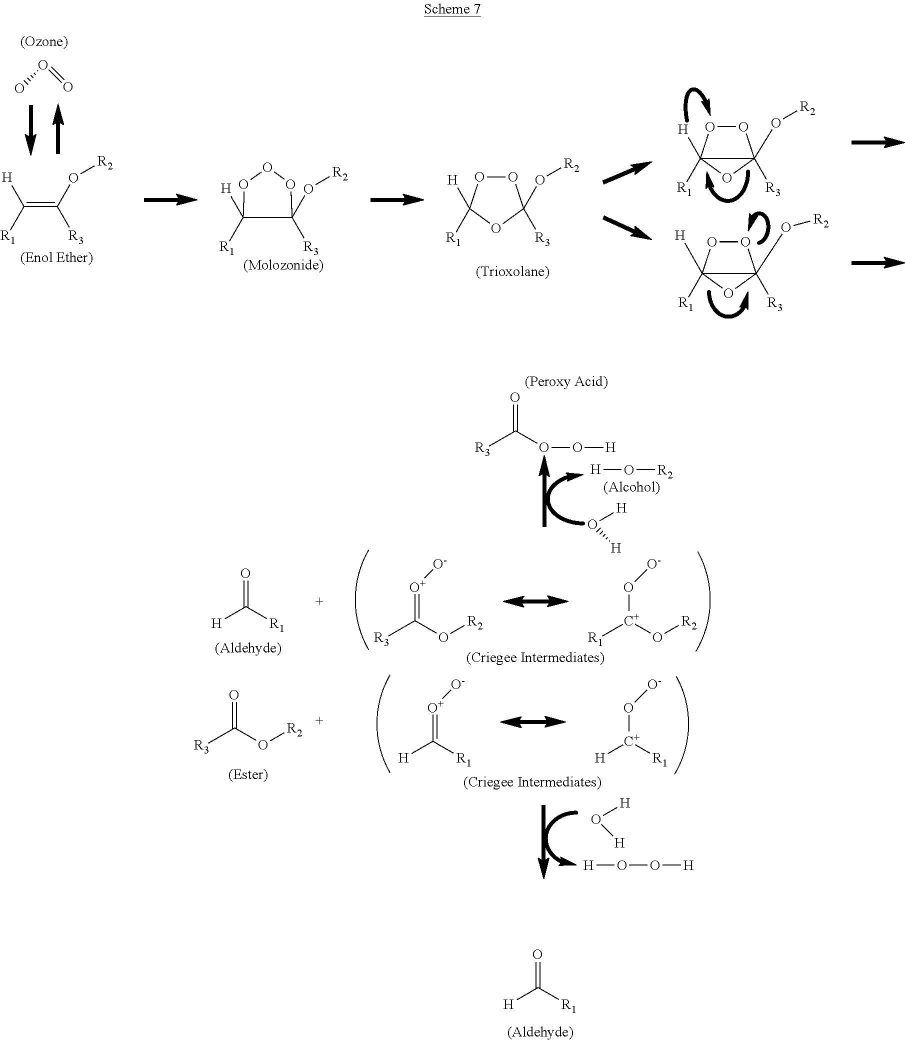

In this enol ether ozonolysis mechanism, an aldehyde is formed, leading to the production of an alcohol and a peroxy acid on reaction of the Criegee intermediate with water (top mechanism), or an ester is formed, leading to the production of an aldehyde with hydrogen peroxide (bottom mechanism). This mechanism would also hold if the enol ether oxygen is replaced with a nitrogen (enamine), sulfur (vinylthioether), a phosphine or another leaving group. Additionally, peroxide formation would also occur with a C═N, C═S, S═S, N═P, C═P, N═N, P═N, P═S, N═S, etc. bond replacing the C═C bond. Additionally, while Scheme 9 shows the trioxolane mechanism, that intermediate can be skipped, and the aldehyde or ester can be formed directly from the molozonide intermediate.

Analogous mechanisms also occur with dienol ethers, divinylthio ethers, dienamines, combinations, etc., with an exemplary dienol ether mechanism provided in Scheme 8.

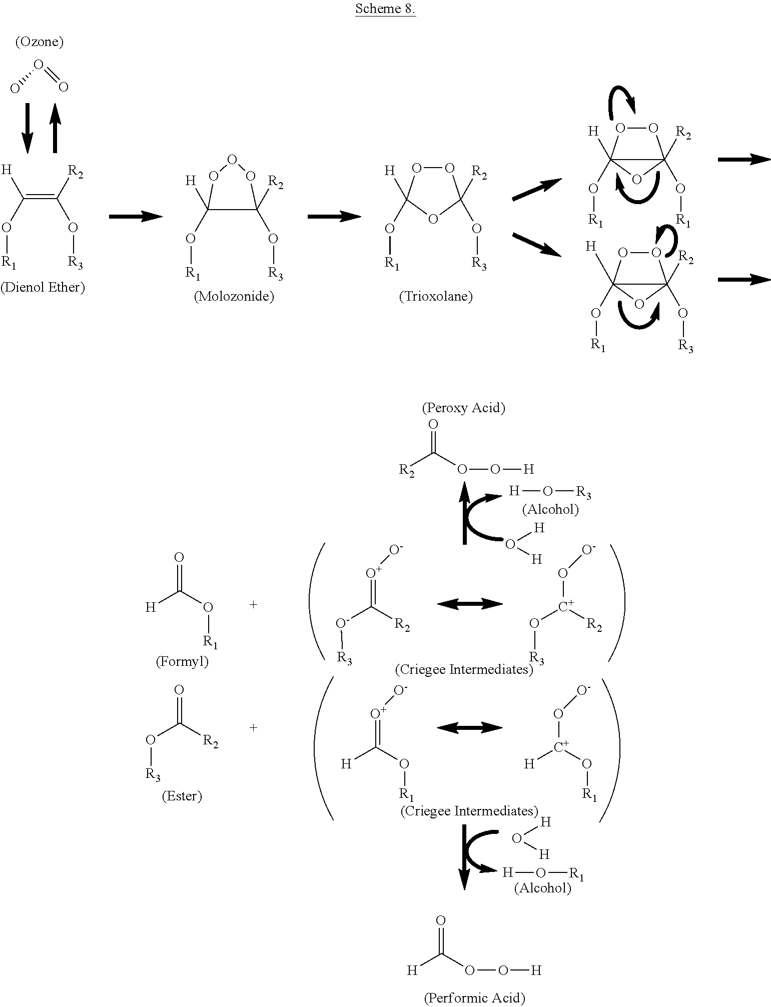

Under Scheme 8, a peroxy acid will be formed in both reactions.

Thus, more generally, the present invention provides a compound that generates a peroxide when it reacts with ozone in the presence of water, the compound comprising the structure I

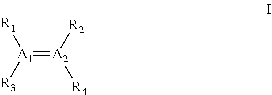

wherein

A₁ and A₂ are independently C, N, S, Si or P;

R₁, R₂, R₃, R₄ are each independently A₃-R₅, a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl;

A₃ is independently O, S, N or P; and

R₅ is a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl. In some of these embodiments, additional R groups bound to A₁ and/or A₂ are present.

In some embodiments, A₁ and A₂ are both C. In other embodiments, at least one of R₁, R₂, R₃, or R₄ is A₃-R₅. This encompasses the following compounds II-VII.

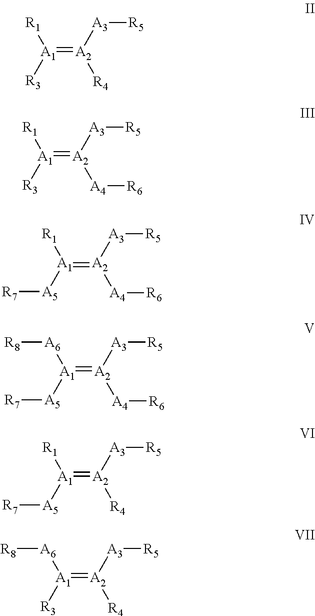

wherein A₄, A₅ and A₆ is each independently O, S, Si, N or P and R₆, R₇ and R₈ is each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

As discussed above, there are two possible products from any ozonolysis reaction, sometimes producing alternative peroxides. The possible product can be influenced by having one or more substituents coming off one of the carbons withdrawing electron density from it, whether through resonance or induction. This will further increase the partial positive charge on one of the carbons, which will help make that particular carbon form the aldehyde or ketone, instead of the unstable Criegee intermediate. Which carbon forms the Criegee intermediate can also be influenced through resonance, since that helps spread out a positively charged, or partial positively charged cation. Therefore, the carbon in an alkene that takes part in some form of resonance would form the Criegee intermediate rather than the other carbon.

Also, by simple induction, not through resonance, the carbon that forms the intermediate can be selected. For example, if there is an electronegative group, or groups, or electron-withdrawing groups attached onto one of the carbons, or part of its substituent group coming off the carbon, then through induction, and electron density being pulled from the carbon, it can be influenced to form the ketone or aldehyde. Furthermore, a carbon in an alkene that has only one alkyl group, and a hydrogen as well, will not have as much electron density being donated to it as a result of the hydrogen atom, so its substituents would not help stabilize the largely positively charged carbon atom in a Criegee intermediate as much as two alkyl groups which is attached to the other side of the alkene group. Therefore, the number of substituent groups on the carbon atoms in an alkene, and the type, whether a hydrogen atom, or a larger chain, or an electron-withdrawing or donating group, or an atom or group that takes part in resonance, will greatly determine which side the Criegee intermediate will form.

More specifically, in compounds II to VII above, A₁ and A₂ can influence where the Criegee intermediate will go. If they are different atoms, the one that is less electronegative, which does not pull electron density as much, would direct the more unstable Criegee intermediate towards it. Also, the atom's size can also play a part, for example, a S or a P are larger than an O, and the larger the atom, the more it is able to spread out charges across the atom, helping to stabilize the partial positive charge of the intermediate. Therefore, larger atoms, and less electronegative atoms would direct the Criegee intermediate towards it. Less electronegative atoms that have lone pairs, such as an nitrogen, also help stabilize charges better as well because they are able to donate electrons easier than more electronegative atoms, such as oxygen atoms. This resonance in between surrounding atoms will help stabilize as well. Some atoms also have extra electron shells that could influence as well and stabilize or destabilize.

A₃-A₆ in compounds II to VII can also help determine where the Criegee intermediate will go, particularly if they are O, S, N or P. These have lone pairs that can donate electron density towards the double bond, allowing the reaction to be sped up. Atoms that are larger, or less electronegative are better at stabilizing the Criegee intermediate as a result of being able to donate electron density easier, thus stabilizing the more partial positive charge formed from the Criegee intermediate. We thus do not need an electron-donating group adjacent to the double bond in order for the double bond to react faster, since resonance can donate electron density from far away, as discussed with the light and electrical embodiments described above.

R₁-R₄ in compounds II to VII also are strong determinants of the stability of the Criegee intermediates. For example, if they have resonating structures in them, such as an aromatic or phenyl group, this resonance would help stabilize the Criegee intermediate, which would influence the intermediate to go to that side of the byproduct after the cleavage reaction. If an aromatic ring that is attached directly to the double bond has an electron donating group attached to it, such as in the ortho or para positions, this will stabilize the Criegee intermediate considerably, and would be a main determinant of where the positive charge Criegee intermediate will want to go. An electron-withdrawing group on the aromatic, particularly on the ortho and para positions, will increase the partial positive charge on the atom in the double bond, making the Criegee intermediate unstable, thus causing the Criegee intermediate to go to the other side. An aromatic ring by itself without substituents attached to the phenyl group would help stabilize the Criegee intermediate as well. Also, electron donating and withdrawing groups, whether through resonance or induction, could not only influence the direction of the Criegee intermediate, but also speed up or decrease the speed of the reaction with ozone, and reactive oxygen species as well, both in the atmosphere and solution.

R₅-R₈ in compounds II to VII would not have as much influence on either the speed of the reaction, or the determinant of which side the Criegee intermediate will go to, since those groups are separated from the alkene by an electron donating group (A₃-A₆). However, they still influence the direction and speed nonetheless. For example, If one of those R groups takes part in resonance, and is connected to the electron donating group in resonance, this will cause, for example, an oxygen atom to want to form an oxonium ion with this group. This will cause a positive charge on the oxygen adjacent to the double bond, which will form a greater partial positive charge on the atom in the double bond, which would make a Criegee intermediate more unstable, and be directed to the other side. If there is an electron-withdrawing group on the resonance structure, such as in the ortho or para positions, this will considerably decrease the chances of a Criegee intermediate forming on its side since it will further increase the partial positive charge on the atom in the double bond, as a result of the oxonium group being extremely electron withdrawing. If there is an electron donating group on the Y resonance structure, then it could actually help influence the Criegee intermediate forming on its side since it might push the oxonium formation to go to the side of the double bond, which would also help speed up the reaction as well, besides helping to influence the Criegee formation on its side. If there is no resonance taking place between the R and the electron donating group, then induction will be a main determinant of which side the Criegee intermediate would form. If there is an electron-withdrawing property part of the alkyl chain in the R group, this would decrease the electron density of this alkyl group, which would slow down the formation of the oxonium group to the double bond. If there is just an alkyl group, without electron withdrawing properties, this would increase induction and electron density donation to the X group, and would increase the formation of the oxonium ion formation towards the double bond, helping to increase the stability of the Criegee intermediate, and thus the formation of the Criegee intermediate on its side.

In some embodiments, the compound that generates a peroxide upon reaction with ozone and water is an enol ether, an enamine, a vinyl thioether, a dienol ether, a dienamine, a divinyl ether, or a combination thereof. In some of those embodiments the compound comprises the structure VII, IX, or X

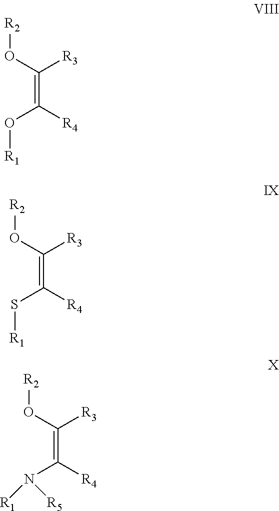

wherein R₁, R₂, R₃, R₄ and R₅ are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

Although the O—R₁, S—R₁ and N—R₁ moieties are shown in the cis configuration, can be also be trans or a mixture. Throughout this specification and claims, unless indicated otherwise, illustrations of a compound that shows such a cis, or conversely a trans, configuration encompass the other configuration, as well as cis-trans mixtures.

In various embodiments of these compounds, the compound comprises the structure XI, XII, XIII, or XIV

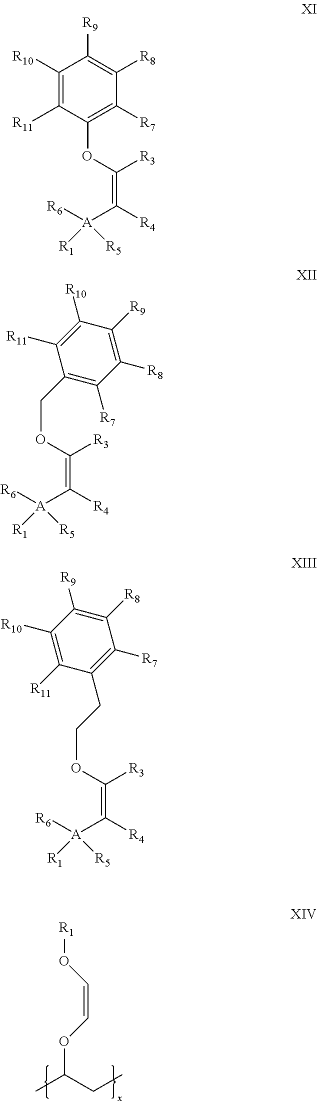

wherein A is O, N, S or P, and R₁, R₃, R₄, R₅, R₆, R₇, R₈ and R₉ are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

Some examples of these compounds comprise the structure XV, XVI, XVII, XVIII, XIX, XX, XXI, XXII, XXIII, or XXIV

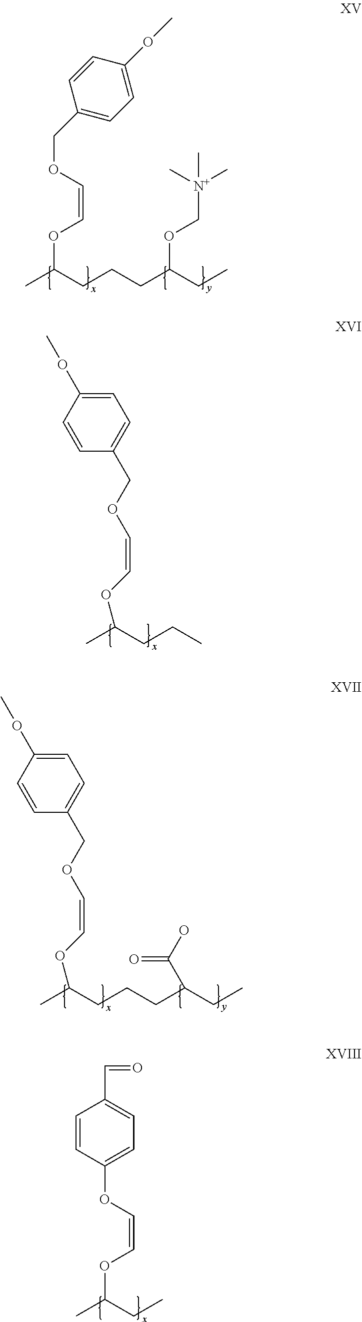

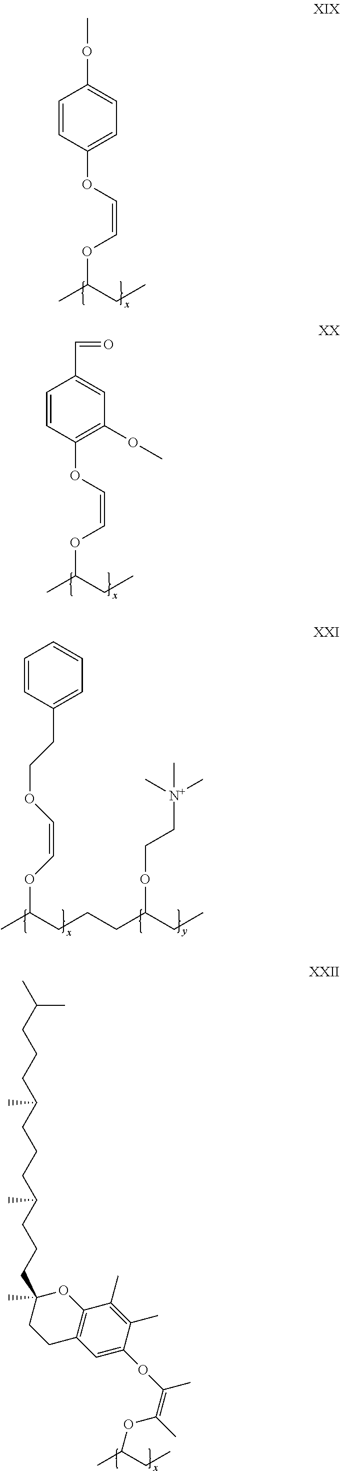

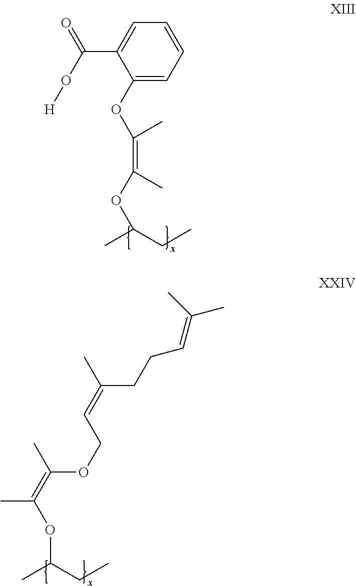

The quaternary ammonium moiety in the copolymer XV above is particularly useful in disinfectants, even without reacting with a free radical, ROS or other reactive species to form a peroxide, because that moiety is similar to other antimicrobial polymers and acts as an antimicrobial itself.

The following additional specific compounds XXV-LIV are provided.

Compound XXV forms a vanillin derivative and performic acid upon ozonolysis. This compound is particularly useful in skin treatment compositions since it protects the skin from ozone and the performic acid ozonolysis product is antibacterial. Additionally, the polymer ozonolysis product is non-toxic.

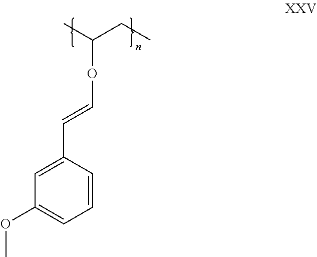

Compound XXVI forms form performic acid and citral and is particularly useful in skin treatment compositions, due to the antibacterial effects of percacetic acid, as well as a coating, e.g., in paint or on a filter, since it provides a pleasant fragrance upon reaction with ozone. Although there are multiple double bonds that are potentially susceptible to reaction with ozone, the double bond in the enol ether moiety is highly favored to be the ozonolysis target due to the increased electron density of that bond, as discussed in U.S. Provisional Application 62/428,137

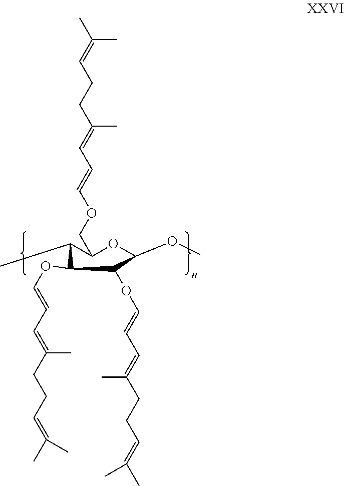

Compound XXVII forms retinyl acetate, peracetic acid, and retinol upon ozonolysis of the enol ether double bond, and is particularly useful in skin treatment compositions, especially for treatment of acne.

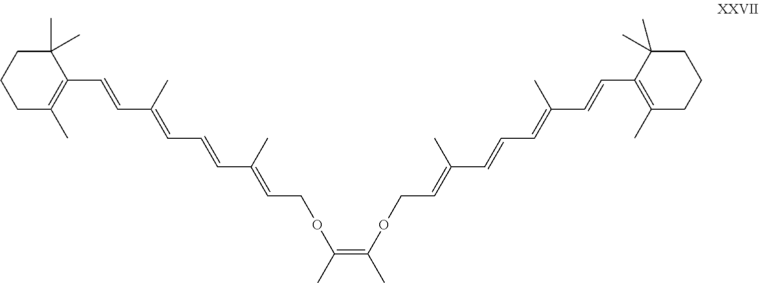 [SMILES: CC1=C(/C=C/C(C)=C/C=C/C(C)=C/CO/C(C)=C(/C)OC/C=C(C)/C=C/C=C(C)/C=C/C2=C(C)CCCC2(C)C)C(C)(C)CCC1]

Upon ozonolysis at the enol ether double bond (highly favored as the ozonolysis target, as discussed immediately above) of compound XXVIII, geranyl acetate, peracetic acid, and geranyl alcohol are formed. This compound is particularly useful as a skin treatment composition as well as a coating, e.g., in paint, in an air freshener, or on a filter.

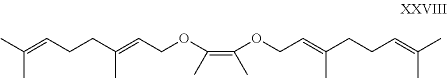 [SMILES: CC(C)=CCC/C(C)=C/CO/C(C)=C(/C)OC/C=C(\C)CCC=C(C)C]

Upon ozonolysis, compound XXIX ortho-phthalaldehyde and performic acid are produced. This compound is particularly useful in cleaning products or coatings since both products are disinfectants.

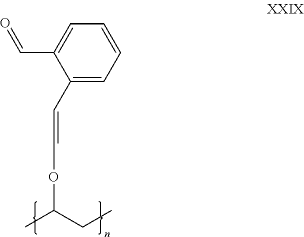

Compound XXX is another compound that is particularly useful in skin care compositions, since, upon ozonolysis, vitamin D and performic acid are formed.

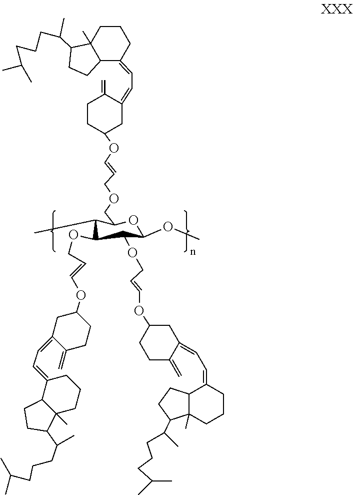

Compound XXXI produces performic acid and vanillin upon ozonolysis. This compound is particularly useful as a skin treatment composition as well as a coating, e.g., in paint, in air fresheners, or on a filter.

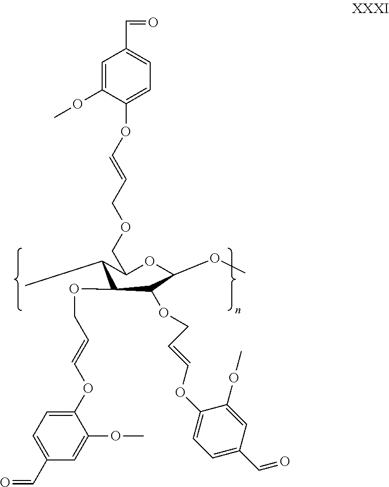

Compound XXXII produces vitamin B6 upon ozonolysis and is therefore particularly useful in skin treatment compositions. The electron-donating groups in the ortho and para positions increases the reaction rate with the double bond.

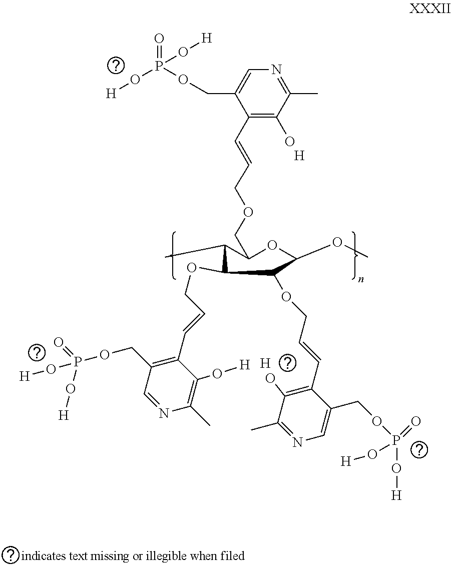

Compound XXXIII, upon ozonolysis, produces performic acid, glucose and cellulose, and is particularly useful as a food or food package coating since all of the products are non-toxic and the compound protects against ozone damage.

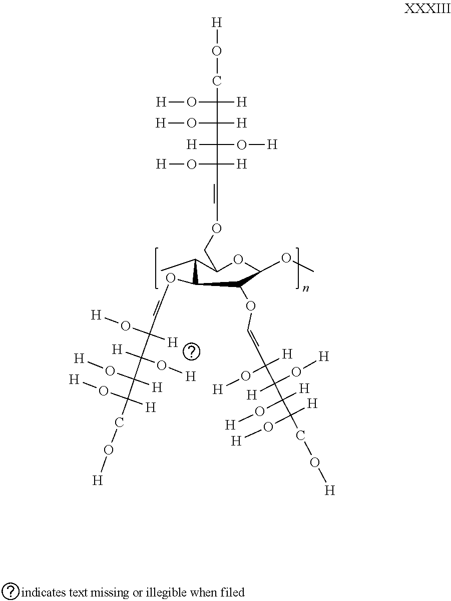

Compound XXXIV produces performic acid and retinal upon ozonolysis, and is therefore particularly useful in skin treatment compositions.

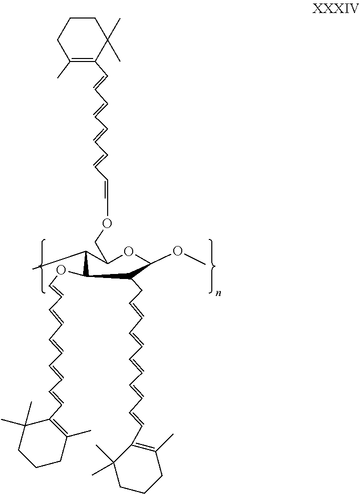

Compound XXXV, upon ozonolysis, is a prodrug that forms vitamin D, peracetic acid, and vitamin D acetate, and is therefore particularly useful in a skin treatment composition, especially as an acne treatment.

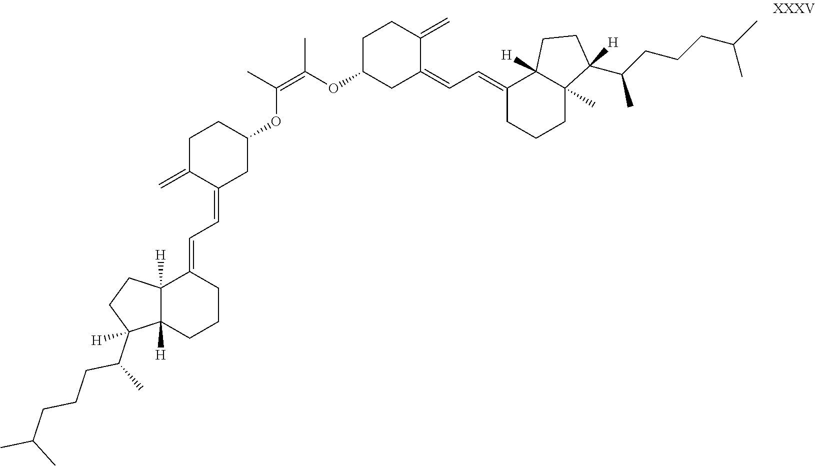 [SMILES: C=C1CC[C@@H](O/C(C)=C(/C)O[C@H]2CCC(=C)/C(=C\C=C3/CCC[C@@H]4[C@@H]([C@H](C)CCCC(C)C)CC[C@@H]34)C2)C/C1=C/C=C1\CCC[C@]2(C)[C@@H]([C@H](C)CCCC(C)C)CC[C@@H]12]

Upon ozonolysis, compound XXXVI produces performic acid, acetyl-coenzyme-A, coenzyme-A and acetone and is particularly useful in a skin treatment composition.

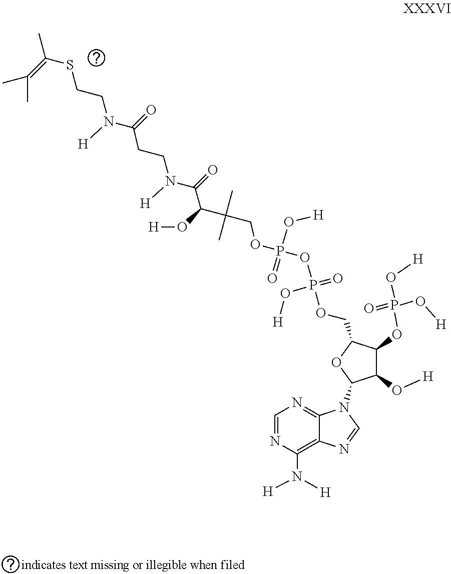 [SMILES: CC(C)=C(C)SCCNC(=O)CCNC(=O)[C@H](O)C(C)(C)COP(=O)(O)OP(=O)(O)OC[C@H]1O[C@@H](n2cnc3c(N)ncnc32)[C@H](O)[C@@H]1OP(=O)(O)O]

Compound XXXVII, upon ozonolysis of the enamine, produces the dye Acid Red 1 and is particularly useful as a coating, e.g., on a filter, where the appearance of the dye shows exposure to ozone.

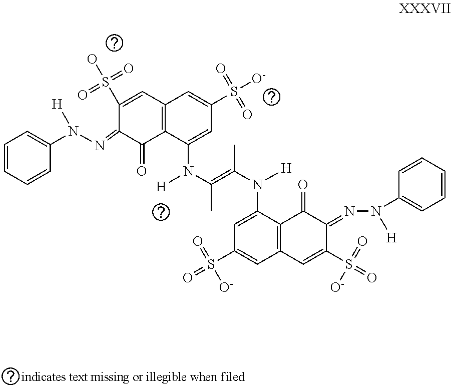 [SMILES: C/C(Nc1cc(S(=O)(=O)[O-])cc2c1C(=O)/C(=N/Nc1ccccc1)C(S(=O)(=O)[O-])=C2)=C(/C)Nc1cc(S(=O)(=O)[O-])cc2c1C(=O)/C(=N/Nc1ccccc1)C(S(=O)(=O)O)=C2]

Upon ozonolysis, compound XXXVIII produces form retinal, performic acid, and an anthracene. The anthracene alters the absorbance of the retinoid moiety by increasing the conjugation of the retinoid, allowing it to absorb light more efficiently in longer wavelengths, helping to stabilize it and be less reactive to UV light.

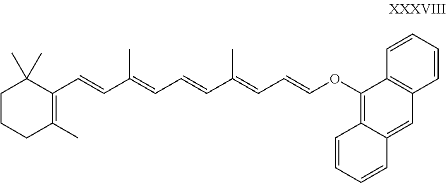 [SMILES: CC1=C(/C=C/C(C)=C/C=C/C(C)=C/C=C/Oc2c3ccccc3cc3ccccc23)C(C)(C)CCC1]

Compound XXXIX is similar to compound XXVIII, but the dienol ether is in the trans position. This compound forms retinyl acetate, retinol, and peracetic acid upon ozonolysis.

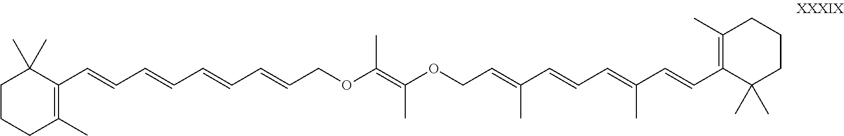 [SMILES: CC1=C(/C=C/C=C/C=C/C=C/CO/C(C)=C(\C)OC/C=C(C)/C=C/C=C(C)/C=C/C2=C(C)CCCC2(C)C)C(C)(C)CCC1]

Compound XL reacts with ozone at the enol ether to form Vitamin D, acetone, and performic acid and is particularly useful in a skin treatment composition.

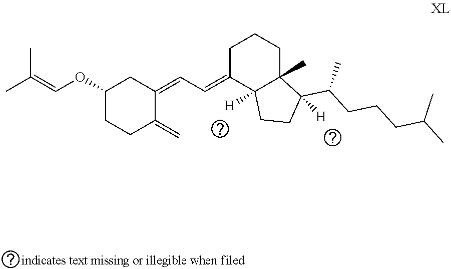 [SMILES: C=C1CC[C@H](OC=C(C)C)C/C1=C/C=C1\CCC[C@]2(C)[C@@H]([C@H](C)CCCC(C)C)CC[C@@H]12]

Compound XLI is similar to compound XL except it produces vitamin D, peracetic acid (not performic acid) and acetone upon ozonolysis.

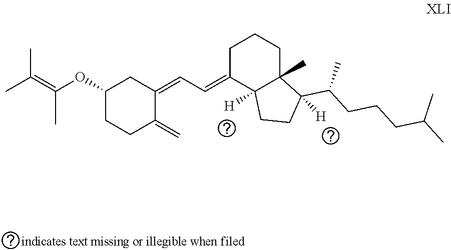 [SMILES: C=C1CC[C@H](OC(C)=C(C)C)C/C1=C/C=C1\CCC[C@]2(C)[C@@H]([C@H](C)CCCC(C)C)CC[C@@H]12]

Compound XLII upon undergoing ozonolysis, provides aspirin, polyvinyl alcohol, and peracetic acid, and is therefore particularly useful in skin care compositions, especially acne treatment and repair mechanisms.

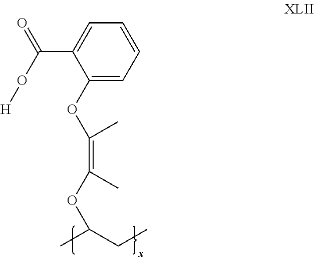

Without being bound by any particular mechanism, this compound would undergo two alternative mechanisms, A and B, as shown in Scheme 9.

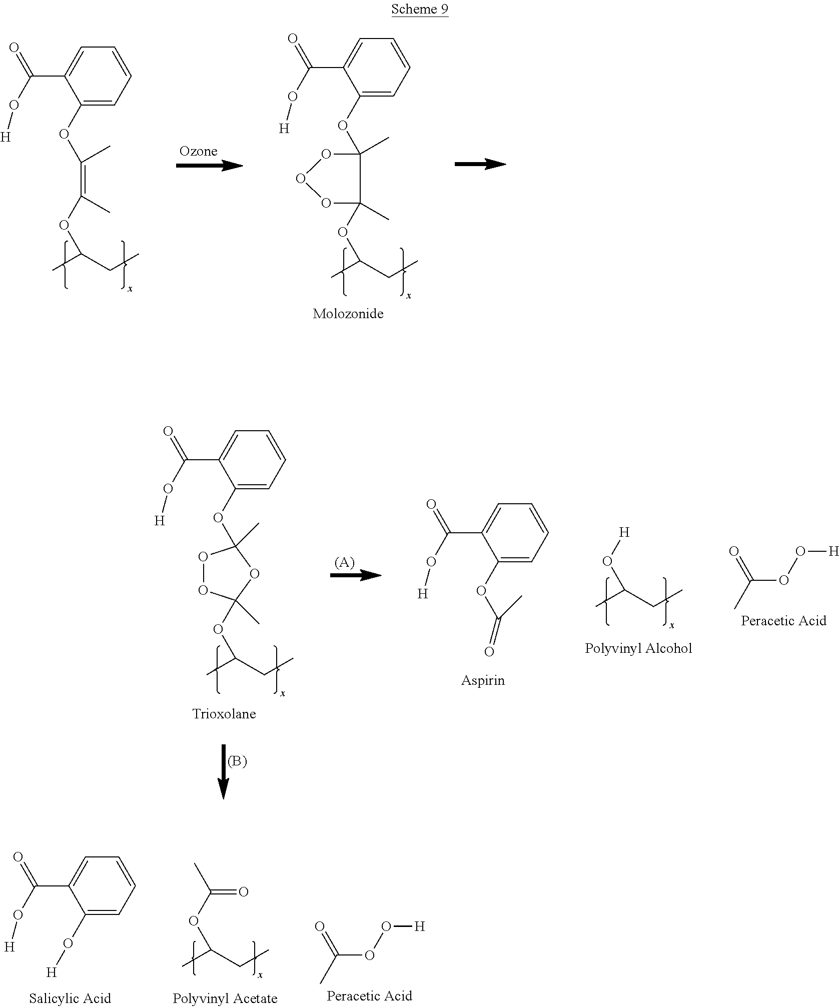

Mechanism A is the predominant mechanism of ozonolysis for this compound, because the aromatic is connected through resonance to the enol ether oxygen atom. In the ortho position on the aromatic ring, there is an electron withdrawing group. This causes the formation of positive charges on its ortho and para positions, which is the carbon adjacent to the oxygen atom in the enol ether. This will cause the formation of an oxonium ion connected to the aromatic ring. This means there is going to be a considerable amount of positive and partial positive charge on the carbon in the alkene closest to the aromatic ring. This will result in a very unstable Criegee intermediate which already has its own large positive partial positive charge. As a result, the Criegee intermediate will primarily be directed towards the side of the alkene closest to the polymer backbone chain. This is Mechanism A, where aspirin and peracetic acid will be formed.

Compound XLIII has the same functional group as compound XLII, but has a cellulose polymer backbone rather than a polyvinyl group.

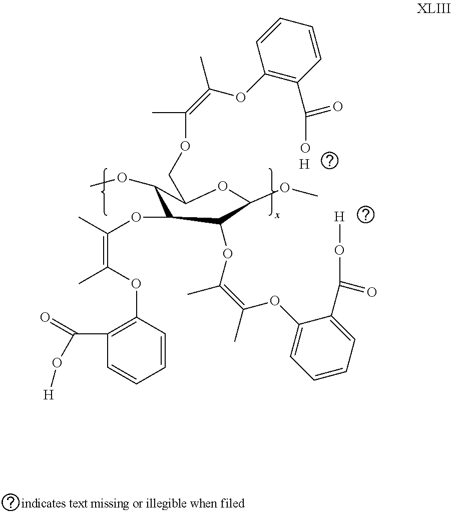

Compound XLIV also has the same functional group as compound XLII, but instead of attaching directly to the cellulose polymer chain, the functional group is attached to hydroxyethyl cellulose, which is easier to attach than compound XLIII because there is less steric hindrance.

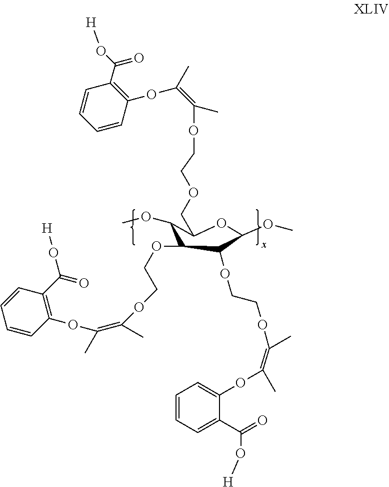

Compound XLV is particularly useful as a prodrug, where the drug is R₁—OH, where the drug is released upon reaction with an ROS (e.g., ozone or superoxide), another reactive species or a free radical. The triphenylphosphonium group targets the mitochondria due to the hyperpolarization of the mitochondrial membrane potential.

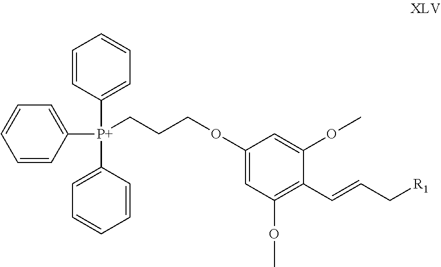

Compound XLVI has three triphenylphosphonium groups so it will target the mitochondria more strongly than compound XLV.

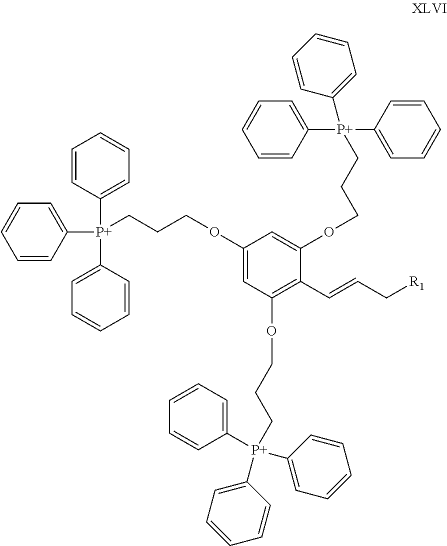

Compound XLVII is a specific example of compound XLVI, that releases camptothecin upon reaction with an ROS or free radical.

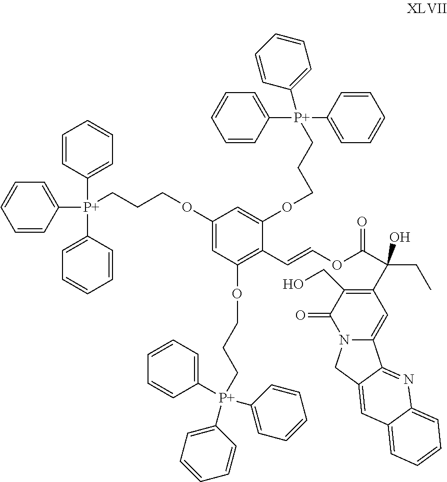 [SMILES: CC[C@@](O)(C(=O)O/C=C/c1c(OCCC[P+](c2ccccc2)(c2ccccc2)c2ccccc2)cc(OCCC[P+](c2ccccc2)(c2ccccc2)c2ccccc2)cc1OCCC[P+](c1ccccc1)(c1ccccc1)c1ccccc1)c1cc2n(c(=O)c1CO)Cc1cc3ccccc3nc1-2]

Compound XLVIII is a prodrug that releases the nucleoside analog gemcitabine upon reaction to an ROS or free radical. This prodrug would be preferably activated in cells experiencing oxidative stress or inflammation, where ROS and free radicals are more prevalent than unstressed cells.

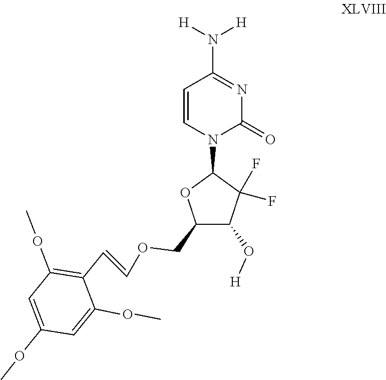 [SMILES: COc1cc(OC)c(/C=C/OC[C@H]2O[C@@H](n3ccc(N)nc3=O)C(F)(F)[C@@H]2O)c(OC)c1]

Compound XLIX produces irinotecan, a camptothecin derivative upon reaction with an ROS, another reactive species, or free radical.

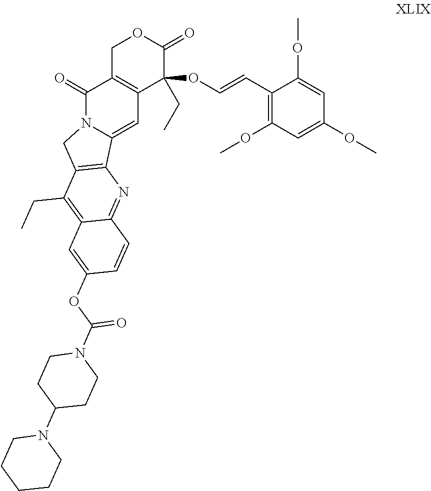 [SMILES: CCc1c2c(nc3ccc(OC(=O)N4CCC(N5CCCCC5)CC4)cc13)-c1cc3c(c(=O)n1C2)COC(=O)[C@@]3(CC)O/C=C/c1c(OC)cc(OC)cc1OC]

Compound L is another example of a prodrug that targets mitochondria and releases camptothecin upon reaction with an ROS or a free radical.

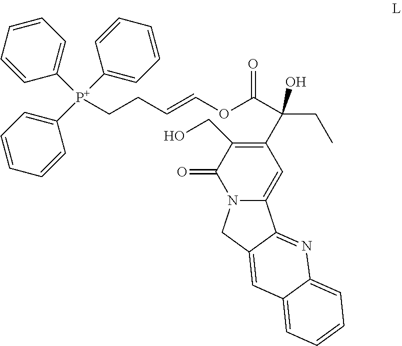 [SMILES: CC[C@@](O)(C(=O)O/C=C/CC[P+](c1ccccc1)(c1ccccc1)c1ccccc1)c1cc2n(c(=O)c1CO)Cc1cc3ccccc3nc1-2]

Compound LI produces a raspberry ketone derivative and performic acid upon reaction with ozone.

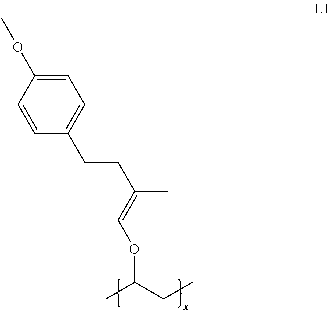

Compound LII produces the fragrance thujone and performic acid upon ozonolysis.

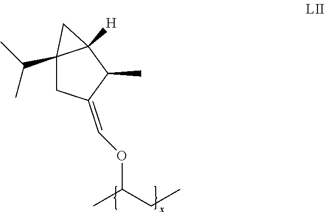

Compound LTII formed geranyl acetate and geraniol (both fragrances) as well as peracetic acid upon ozonolysis.

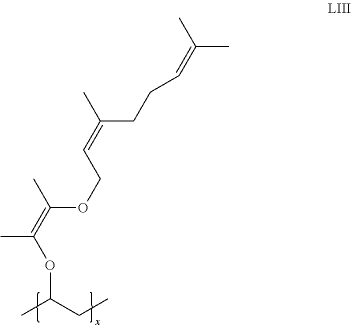

Compound LIV produces vitamin E (tocopherol) and tocopherol acetate as well as peracetic acid, and is therefore particularly useful in skin care compositions, especially acne treatments.

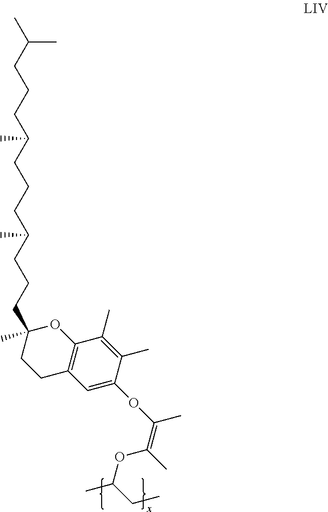

Additionally, other compounds provided in this specification will produce a peroxide on reaction with ozone. Based on the discussion below, the skilled artisan would understand that all of the above compounds also react with, and degrade, other ROS, other reactive species, and free radicals, but may not necessarily generate a peroxide. The skilled artisan can also determine, without undue experimentation, which additional compounds provided in this specification will generate a peroxide on reaction with ozone, another ROS, other reactive species, or free radicals.

### Alkynes

The present invention is also directed to alkyne compounds reactive with a free radical, a reactive oxygen species (ROS) or another reactive species. The compounds comprise internal alkynes having the structure LV, LVI or LVII

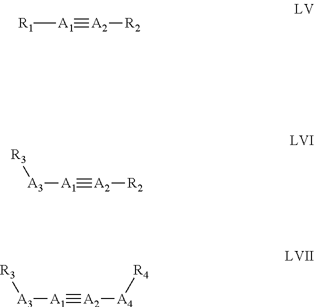

wherein A1, A2, A3, and A4 is each independently C, O, S, N or P and R1, R2, R3, and R4 is each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

These compounds, having internal alkynes, are more effective in reacting with free radicals like nitrogen oxides than alkenes, since free radicals can abstract a hydrogen atom from an alkene double bond (Pryor et al., 1982), whereas an internal triple bond, as here, does not have a hydrogen to be abstracted by the free radical, so the reactivity of alkynes with free radicals is not potentially inhibited by hydrogen abstraction. Additionally, since these compounds do not abstract a hydrogen, they will not produce nitric acid from the reaction with nitrogen oxides.

Scheme 10 shows a general mechanism of the chemistry that takes place with an alkyne, because it is so reactive, we can now target more reactive species and air pollutants in the air, and have them covalently attached to the alkyne, and then have an alcohol or some other functional group fall off upon reaction. The final product with the NO₂ and SO₂ can also have a double or single bond rather than a triple bond. We can speed the falling off of the group by making a good leaving group (some electron withdrawing and stabilization and resonance properties). This will allow us to make pure molecules upon reaction with various reactive species. As shown in Wilkinson (2015), slide 11, the triple bond has increased electrostatic potential energy over the corresponding double bonds, so the triple bond compounds will react better with lower energy air pollutants. Thus, the reactivity of pollutants such as NOx and SOx with alkynes is greater than with alkenes, for example as shown in Prior (1982), Scheme 1 therein, on p. 6690.

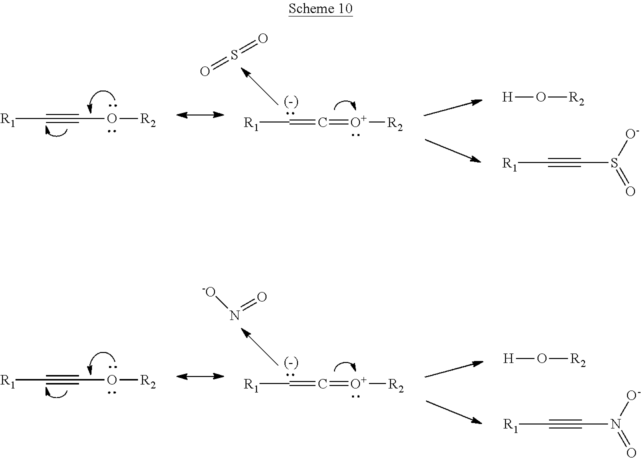

Thus, the alkynes provided herein are useful to target lower energy molecules such as nitrogen dioxide, sulfer dioxide, etc. As with other molecules herein and in PCT/US2016/052529 and WO/2016/023015, and more fully described below, the alkynes can be designed to, upon reaction, produce a nontoxic or useful compound, by having that compound be incorporated into the moiety analogous to the R₁ or R₂ in Scheme 10. The reaction favorability can also be modulated by including enol ether, enamine, vinyl thioether resonance-enhancing or reducing moieties or combinations of any of those, as further elaborated below. The alkyne compounds can also be designed to produce peroxides, as with the compounds described above.

As with other compounds described herein, in some embodiments, these alkyne compounds yield a useful product upon reaction with a free radical, an ROS or another reactive species. In other embodiments, the reaction produces no useful product.

Examples of specific alkynes that undergo ozonolysis as well as reactions with other ROS, another reactive species and free radicals are compounds LVIII, LIX, and LX.

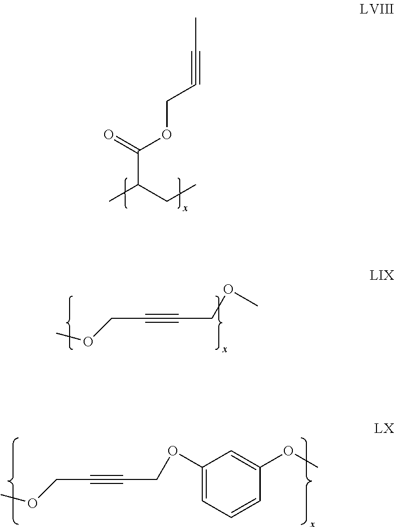

where X is an integer between 1 and 1,000,000.

Particularly useful alkyne general structures are LXI, LXII, LXIII or LXIV

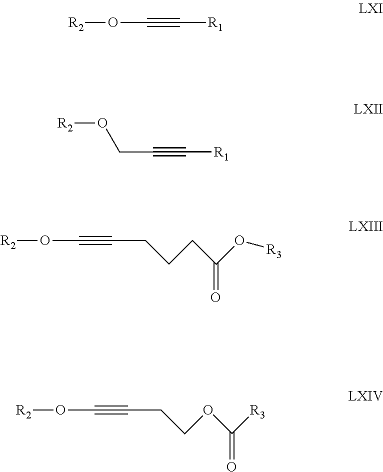

wherein R₂ and R₃ is each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

Specific examples of those compounds and compounds where nitrogen substitutes for an oxygen are LXV, LXVI, LXVII, LXVIII, LXIX, LXX, LXXI, LXXII, LXXIII, LXXIV, LXXV, LXXVI, LXXVII, LXXVIII, LXXIX, LXXX, and LXXXI

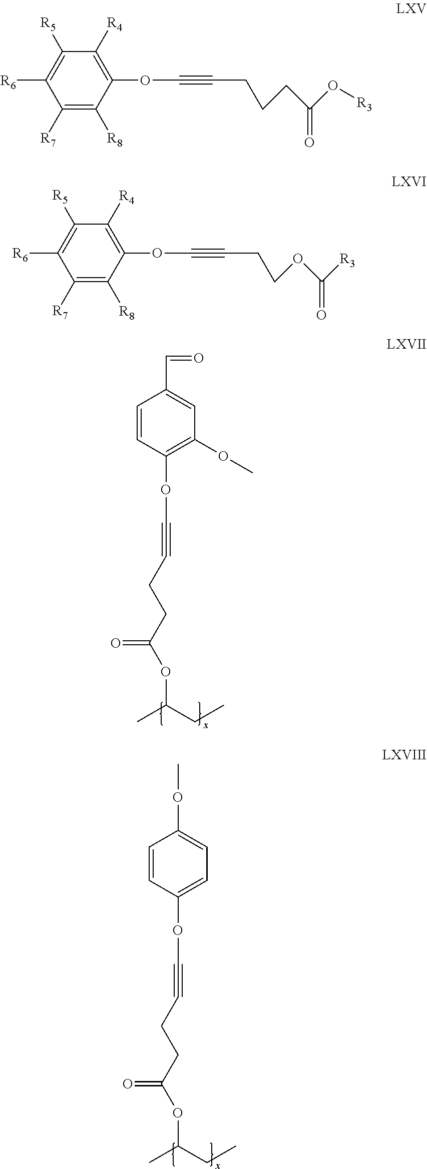

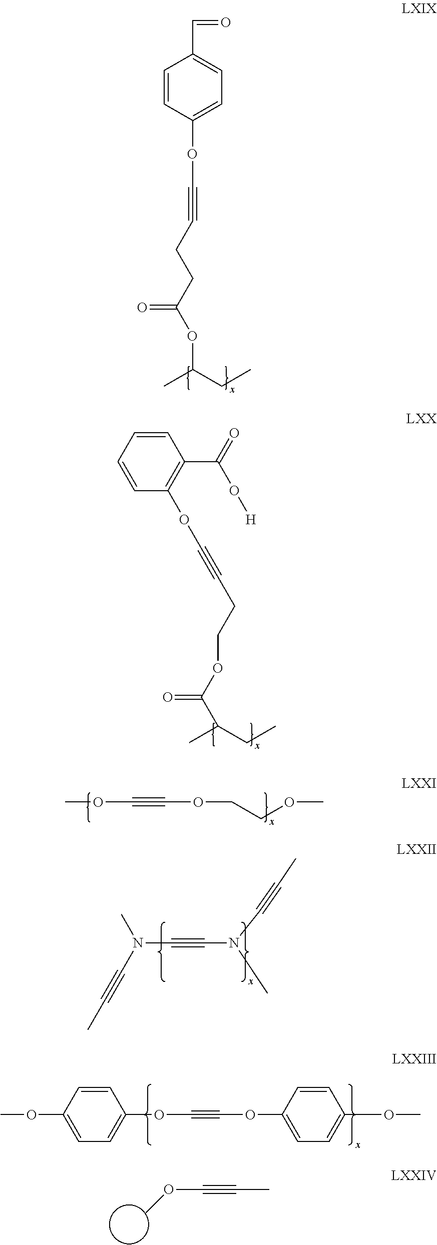

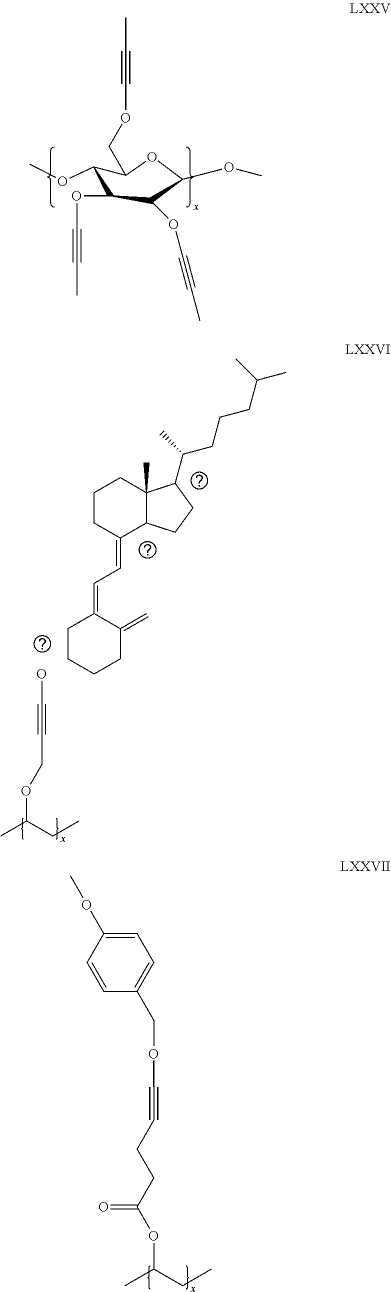

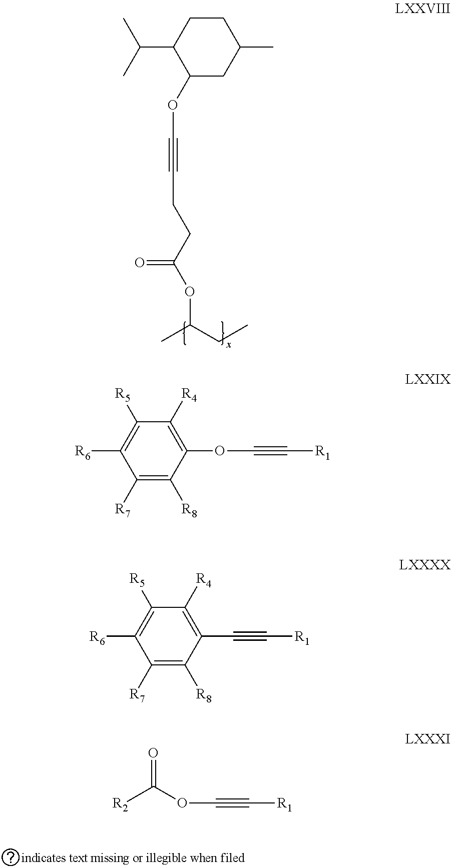

wherein X is an integer from 1 to 1,000,000, the large circle of LXXIV is a particle, and R₁, R₂, R₄, R₅, R₆, R₇ and R₈ is each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

### Enol Ethers, Enamines and Vinyl Thioethers

The present invention is also based on the discovery that enol ethers, enamines, and vinyl thioethers can be used to decompose free radicals, ROS, and other reactive species, and to activate inactive compounds.

Enol ethers, enamines, and vinyl thioethers

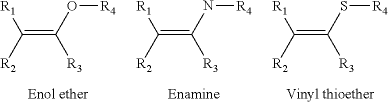

react with free radicals ROS and other reactive species to hydrolyze the R₄ moiety to form R₄OH, R₄NH₂ or R₄SH (U.S. Pat. No. 2,773,072; Stadelmann-Ingrand et al., 2001; US Patent Publication 2003/0026841; European Patent EP1102785B1). Those compounds also react with ROS including ozone. The present invention provides compounds that utilize these reactions to activate inactive compounds and to terminate deleterious free radical and ROS chain reactions.

Thus, in some embodiments, a compound reactive with a free radical, a reactive oxygen species (ROS) or another reactive species is provided. The compound comprises LXXXII, LXXXIII, LXXXIV, LXXXV, or LXXXVI

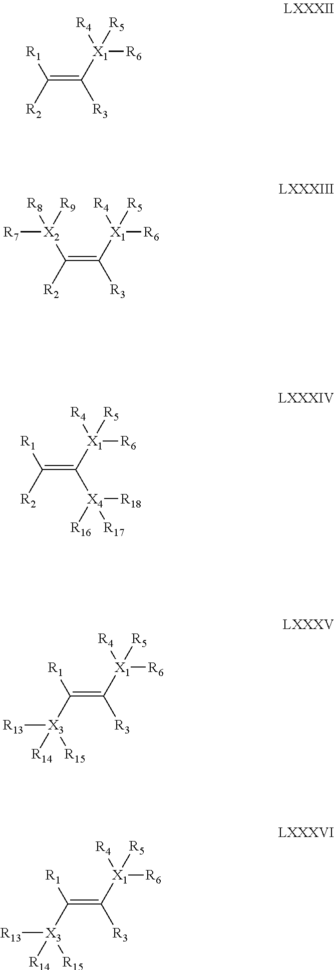

wherein

X₁, X₂, X₃ and X₄ are each independently O, N, P or S; and

R₁-R₁₈ are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl, provided R⁴ is not H or a lone pair of electrons.

Any free radical would cause the above free radical reaction. In various embodiments, the free radical is superoxide, hydrogen peroxide, a hydroxyl radical, nitric oxide, peroxynitrite, an organic radical, a peroxy radical, an alkoxy radical, a thiyl radical, a sulfonyl radical, or a thiyl peroxyl radical.

The ROS reactions of the invention compounds react as those described in PCT Patent Application PCT/US2016/052529 and PCT Patent Publication WO/2016/023015, both incorporated by reference in their entirety. Those references utilize ozone as an exemplary ROS. However, any ROS would cause the above ROS reaction. In various embodiments, the ROS is singlet oxygen, dioxygen, triplet oxygen, ozone, nitrogen oxides, ozonide, dioxygenyl cation, atomic oxygen, sulfur oxides, ammonia, carbon monoxide, hydrogen peroxide, organic hydroperoxides, nitrosoperoxycarbonate anion, nitrocarbonate anion, hypochlorous acid, dinitrogen dioxide, nitronium, atomic oxygen, or hydroxyl anion.

Thus, without being bound to any particular mechanism, the compounds of the present invention are hydrolyzed by reaction with free radicals, acids, ROS and other reactive species.

The present invention thus provides compounds that can react with free radicals, ROS or other reactive species to prevent those reactive species from having a deleterious effect in the environment or in a biological system such as the human body. For example the compound V above has four free radical-, ROS— or other reactive species-reacting moieties, and, by joining any of the individual units LXXXII-LXXXVI into a polymeric compound, e.g., as in compound XCVIII, CVI, CI, and CIII-XCIX, below, hundreds of free radical- or ROS-reacting moieties can be provided in one compound.

Naturally occurring enol ether compounds protect biological systems, including humans from deleterious effects of free radicals, ROS and other reactive species. For example, beneficial effects of plasmalogens are due to the extremely fast enol ether bond reactivity with ROS, thus preventing unwanted ROS and other reactive species reactions with other vulnerable unsaturated molecules, lipids, fatty acids, or even DNA. For example, deficiencies of plasmalogens in the brain are common themes in Alzheimer's disease, Parkinson's disease, Down syndrome, and others (Braverman et al., 2012; Sindelar et al., 1998). In plasmalogens, the enol ether bond acts as a “sacrificial oxidant” because the hydrogen atoms on the vinyl ether bond have “relatively low disassociation energies and are preferentially oxidized over diacyl GP when exposed to various free radicals and singlet oxygen. Plasmalogens are consumed in this reaction. This was proposed to spare the oxidation of polyunsaturated fatty acids and other vulnerable membrane lipids”. Id. In other words, the enol ether bond reacts much quicker than regular double bonds, so it protects those regular double bonds from oxidation.

Additionally, free radicals (hydroxyl radicals and superoxide) and reactive species cause the formation of an alcohol and an α-hydroxy aldehyde. Without being bound by any particular mechanism in this application, the alcohol/α-hydroxy aldehyde formation is proposed to be through an epoxide mechanism (Stadelmann-Ingrand, 2001—see scheme 1 on p. 1270). According to that model, singlet oxygen forms an aldehyde with an (n−1) carbon chain length via a dioxetane intermediate mechanism. In other words, non-radicals such as ozone and singlet oxygen break the plasminogen enol ether double bond in half to produce either aldehydes or formyls. Radicals form an alcohol, and both carbons on the double bond are still attached, with an α-hydroxyl group being formed as well on the aldehyde.

Besides providing compounds that react with free radicals, ROS and other reactive species to prevent deleterious reactions, i.e., a free radical or ROS or other reactive species scavenger, any of the products of the reactions discussed in this specification can be an active agent, e.g., a pharmaceutical, nutrient, fertilizer, biocide, etc., as described below and in PCT/US2016/052529 and WO/2016/023015. Hybrid molecules, providing multiple different useful products in a monomeric, oligomeric or polymeric compound, under any one or combination of the above pathways, are also envisioned.

It is noted that the enol ethers, enamines, and vinyl thioethers that react as above do not have a stabilized double bond, for example as on vanillin (below), where the double bond is stabilized by the aromatic ring:

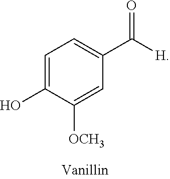

By contrast, the compounds of the instant invention have a reactive double bond, allowing reaction with free radicals, strong acids, ROS or other reactive species.

The compounds provided in this specification can be monomeric, generally having a molecular weight less than 1000, for example compounds LXXXVII-XCIV, C and CII below, or oligomeric or polymeric, generally having a molecular weight more than 1000, for example compounds XCVIII, CVI, CVII, and CIII-XCIX.

Also as discussed above, in some embodiments, the compounds provided in this specification are converted into an active agent (i.e., a useful compound) after reacting with the free radical, an ROS or another reactive species, depending on the design of the compound, as discussed above. Such active agents can have the form

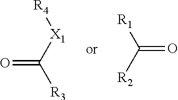

when the compound is designed to create the active agent upon reaction with oxone or singlet oxygen. Alternatively, the active agent has the form

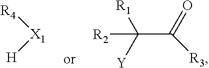

where Y is derived from the free radical, other reactive species, acid or the reaction (e.g., a hydroxyl from water), when the compound is designed to create the active agent upon reaction specific reactive species depending on the conditions, environment (e.g., particular cells or organelles) and whether exposed to the atmosphere or solution.

The compounds and/or the active agents provided throughout this specification are not limited to having any particular physical properties. For example they can be volatile (e.g., as a biomarker) or non-volatile in air, or fully water soluble, sparingly water soluble or non-water soluble, lipophilic or hydrophilic, and charged or uncharged.

In various embodiments, the active agent is a biocide. In some embodiments, the biocide is a pesticide, e.g., a fungicide, an herbicide, an insecticide, an algicide, a molluscicide, a miticide, a repellants, or a rodenticide.

In other embodiments, the biocide is an antimicrobial, e.g., a germicide, an antibiotic, an antibacterial, an antiviral, an antifungal, an antiprotozoal, or an antiparacidal. The antimicrobial can be formulated and utilized as a pharmaceutical or for environmental administration, e.g., inside or outside, and not applied directly to a human or animal. When the antimicrobial is used in the environment, it can be formulated in any form, for example as a paint or a spray, or integrated into a solid material, or coated on the surface of a solid material.

Nonlimiting examples of biocides are (S)-3-anilino-5-methyl-5-phenylimidazolidine-2,4-dione, 1,4-nonyl lactone, 1,4-undecanolide, I-naphthyl-n-methylcarbamate, 2-(1-methylpropyl)phenyl methylcarbamate, 2-(m-chlorophenoxy)propionamide, 2,4-d, 20-hydroxyecdysone, 2-imidazolidone, 2-undecanone, 3′-(trifluoromethyl)acetophenone, 3-hydroxycarbofuran, 3-ketocarbofuran, abamectin, acephate, acetochlor, acetogenins, acetylacetone, acibenzolar-s-methyl, acrinathrin, alachlor, alanycarb, aldicarb, aldicarb-sulfone, aldicarb-sulfoxide, aldoxycarb, allethrin, amicarbazone, amidosulfuron, aminobenzaldehydes, aminocarb, amphotericin b, azadirachtin, azafenidin, azamethiphos, azimsulfuron, azinphos-ethyl, azinphos-methyl, azoxystrobin, barban, benalaxyl, benalaxyl-m, benazolin, benazolin-ethyl, bendiocarb, benodanil, benomyl, benoxacor, bentazon, benzadox, benzaldehydes, benzofenap, benzoin, benzoximate, benzoylureas, bifenazate, bifenthrin, bilanafos, binapacryl, bioallethrin, bioresmethrin, bistrifluron, bixafen, blasticidin s, boscalid, brodaifacoum, bromacil, bromadiolone, bromobutide, bromopropylate, bufencarb, buprofezin, butafenacil, butocarboxim, butoxycarboxim, butoxypropyl ester, caffeine, camphor, capsaicin, captafol, captan, carbaryl, carbendazim, carbetamide, carbofuran, carbofuran-3-keto, carbosulfan, carboxin, carboxine, carpropamid, carvone, chloranil, chiorantraniliprole, chlorbromuron, chlorbufam, chlorfluazuron, chlorimuron ethyl ester, chlorobenzilate, chlorogenic acid, chlorophacinone, chloropropylate, chlorotoluron, chloroxuron, chlorpropham, chlorsulfuron, chlortoluron, chlozolinate, chromafenozide, cinerin, cinnamaldehyde, cinnamyl acetate, cinosulfuron, cis-1,2,3,6-tetrahydrophthalimide, cismethrin, cis-mevinphos, cis-permethrin, citral, citronellal, clethodim, clodinafop-propargyl, cloethocarb, clofencet, clomazone, clomeprop, cloquintocet-mexyl, coumaphos, coumarins, coumatetralyl, crotoxyphos, cyantraniliprole, cyclanilide, cycloheximide, cyclosulfamuron, cycloxydim, cycluron, cyflufenamid, cyfluthrin, cyhalothrin, cymoxanil, cyperethrin, cypermethrin, cyphenothrin, daimuron, daminozide, daptomycin, deet, deguelin, deltamethrin, derris (rotenone), desmedipham, desmethyl-formamido-pirimicarb, dialifos, dibutyl adipate, dichlone, dichlormid, dichlorobenzophenone, diclocymet, diclomezine, dicrotophos, diethofencarb, difenacoum, difenoxuron, difethialone, diflubenzuron, diflufenican, diflufenzopyr, dihydro-5-heptyl-2(3h)-furanone, dihydro-5-pentyl-2(3h)-furanone, dimefluthrin, dimefuron, dimethachlor, dimethenamid, dimethoate, dimethomorph, dimethyl fumarate, dimethyl phthalate, dimetilan, dimoxystrobin, dinobuton, dinocap, dinoterbon, dioxacarb, diphacinone, dipropyl isocinchomeronate, ditalimfos, dithianon, diuron, doramectin, d-phenothrin, drazoxolon, emamectin benzoate, empenthrin, encainide, endrin aldehyde, endrin ketone, eprinomectin, esfenvalerate, ethienocarb, ethiofencarb, ethirimol, ethoxysulfuron, ethyl formate, etobenzanid, famoxadone, fenamidone, fenethacarb, fenfuram, fenobucarb, fenoxacrim, fenoxanil, fenoxaprop ethyl ester, fenoxycarb, fenpropathrin, fenpyroximate, fenuron, fenvalerate, flamprop-isopropyl, flazasulfuron, flocoumafen, flonicamid, fluazifop-p-butyl, fluazolate, fluazuron, flubendiamide, flucycloxuron, flucythrinate, flucytosine, flufenacet, flufenoxuron, flumethrin, flumioxazin, flumipropyn, flumorph, fluometuron, fluopicolide, fluopyram, fluoroacetamide, fluoroimide, fluoroquinolones, flupoxam, flupropacil, flupyrsulfuron, fluquinconazole, fluridone, flurochloridone, fluroxypyr-meptyl, flurtamone, flutolanil, fluxapyroxad, folpet, foramsulfuron, forchlorfenuron, formaldehyde, formetanate, formothion, fosmethilan, fosthiazate, fthalide, furametpyr, furathiocarb, furazolidone, furethrin, furfural, furilazole, glyphosate, glutaraldehyde, griseofulvin, halacrinate, halofenozide, halosafen, haloxyfop methyl ester, hexaflumuron, hexazinone, hexythiazox, hydranal, hydroprene, icaridin, iclosamide, imazamox, imazapic, imazapyr, imazaquin, imazethapyr, imazosulfuron, imiprothrin, inabenfide, indandiones, indanofan, indoxacarb, iprodione, iprovalicarb, isocarbophos, isofenphos, isoprocarb, isoprothiolane, isoproturon, isopyrazam, isotianil, isoxachlortole, ivermectin, jasmolin i,ii, kresoxim-methyl, lactofen, lenacil, linuron, lufenuron, lythidathion, malathion, mandipropamid, mecarbam, mefenacet, mefluidide, mepronil, meptyldinocap, mesotrione, metaflunmizone, metalaxyl, metamitron, meta-phthaldialdehyde, metazachlor, methabenzthiazuron, methasulfocarb, methfuroxam, methidathion, methiocarb, methomyl, methoxyfenozide, metobromuron, metofluthrin, metolachlor, metolazone, metolcarb, metominostrobin, metoxadiazone, metoxuron, metrafenone, metribuzin, molinate, monolinuron, monuron, morfamquat, myclozolin, naftalofos, naphthaleneacetamide, naproanilide, naptalam, neburon, neem (azadirachtin), nicosulfuron, nitrobenzaldehydes, nitrofurantoin, norcotinine, norflurazon, novaluron, octanone, octhilinone, ofurace, omethoate, ortho-phthaldialdehyde, orysastrobin, oxadiargyl, oxadiazon, oxadixyl, oxamyl, oxasulfuron, oxaziclomefone, oxolinic acid, oxycarboxin, oxytetracycline, oxythioquinox, para-phthaldialdehyde, pencycuron, penflufen, penthiopyrad, permethrin, phenisopham, phenmedipham, phenothrin, phenserine, phenthoate, phosalone, phosdrin, phosmet, phosphamidon, phosphocarb, phthalaldehydes, phthalamic acid, phthalates, phthaldialdehydes, phthalide, picaridin, pilsicainide, pindone, piperitone, pirimicarb, prallethrin, pretilachlor, prochloraz, procymidone, prohexadione, promecarb, pronamide, propachlor, propamocarb, propaquizafop, propetamphos, propham, propoxur, proquinazid, prosulfuron, pymetrozin, pymetrozine, pyracarbolid, pyraclostrobin, pyrazolynate, pyrazon, pyrazophos, pyresmethrin, pyrethrin, pyrethroids, pyribencarb, pyridaben, pyridaphenthion, pyridate, pyrinuron, pyroquilon, quinacetol, quinoclamine, rafoxanide, ralfinamide, rimsulfuron, rivastigmine, rotenone, safinamide, s-bioallethrin, scilliroside, sedaxane, sethoxydim, siduron, sintofen, sordarin, spinosad, spinosyn d, spiromesifen, spirotetramat, streptomycin, strychnine, sulcotrione, sulfentrazone, tebufenozide, tebufenpyrad, tebuthiuron, tecloftalam, teflubenzuron, telithromycin, tepraloxydim, terallethrin, terbacil, terbucarb, terephthalaldehyde, tetramethrin, tetranortriterpenoid, thenylchlor, thiacloprid-amide, thidiazimin, thidiazuron, thifluzamide, thiofanox, tiadinil, tocainide, tolfenpyrad, tolperisone, tralkoxydim, tralomethrin, transfluthrin, trans-mevinphos, trans-permethrin, triadimefon, triasulfuron, triazamate, triazofenamide, trichloroisocyanuric acid, trifloxysulfuron, triflumuron, triforin, triforine, trimethacarb, trinexapac-ethyl, valifenalate, vamidothion, vinclozolin, warfarin, ylachlor, and zoxamide.

The effectiveness of these compounds can be tested by any means known in the art. In some embodiments, an inactive antibacterial compound can be tested for the release of the activated compound by spotting the inactive compound on a bacterial lawn, e.g., in a petri dish, in the presence and absence of ROS, another reactive species, free radicals or strong acid, where, with an inactive compound that effectively reacts with free radicals to release the active antibacterial compound, the bacteria around the ozone reacting compound are killed but the bacteria around the compound where ozone is absent will not be killed.

In further embodiments, the active compound is a nontoxic useful compound, such as a cosmetic or a fertilizer, e.g., urea. An inactive compound that provides a fertilizer such as urea after exposure to free radicals, ROS or another reactive species in the air or soil would provide a slow release fertilizer, which would require fewer applications, and potentially avoid fertilizer runoff, providing less fertilizer loss and environmental contamination, than standard fertilizer.

In these embodiments, the fertilizer can be released from an inactive compound that is a small molecule or polymer. The inactive compound can also be cationic, which would be held in soils that have significant cation exchange capacity, thus further avoiding loss of fertilizer by runoff. The cation could also be designed to have antimicrobial properties.

In additional embodiments, the active agent is a pharmaceutical. In these embodiments, the compound, in a pharmaceutical composition, may be administered locally and/or systemically. As used herein, the term “local administration” is meant to describe the administration of a pharmaceutical composition of the compound to a specific tissue or area of the body with minimal dissemination of the composition to surrounding tissues or areas. Locally administered pharmaceutical compositions are not detectable in the general blood stream when sampled at a site not immediate adjacent or subjacent to the site of administration.

As used herein the term “systemic administration” is meant to describe in vivo systemic absorption or accumulation of drugs in the blood stream followed by distribution throughout the entire body. Administration routes which lead to systemic absorption include, without limitation: intravenous, subcutaneous, intraperitoneal, transdermal, inhalation, oral, intrapulmonary and intramuscular. The pharmaceutical can be used anywhere a strong acid, free radicals, ROS or another reactive species are available to react with the inactive compound to form the active agent. Examples include the bloodstream, GI tract, oral administration, intramuscular, intraperitoneal, intranasal, etc Further, the pharmaceutical can be used to treat any disease, e.g., cancer, cardiovascular diseases, neurological disorders, autoimmmune diseases, viral and bacterial diseases, cystic fibrosis, inflammatory diseases, etc.

The pharmaceutical is formulated such that an effective dose of the active agent is provided after administration and exposure to a strong acid, a free radical, an ROS or another reactive species (depending on the design of the compound) at the site of activation. Thus, the administration of an effective dose of a particular active agent would require a greater dose of the inactive compound if administered to a site that has a low level of ROS (e.g., internal tissues), another reactive species, acid, or free radicals (e.g., a building wall) than if administered to a site that has a higher level of ROS, another reactive species (e.g., the air, the lungs or the skin), acid (stomach lumen) or free radicals (e.g., inflammatory tissue). Also, ROS and reactive species can vary inside organelles. For example, the mitochondria has much higher levels of ROS and reactive species compared to other organelles and the cytosol. Therefore, the mitochondria is a great target that contains concentrated ROS that can activate the agent.

Pharmaceutically acceptable carriers for formulation of the inactive compound may be covalently or non-covalently bound, admixed, encapsulated, conjugated, operably-linked, or otherwise associated with the inactive compound such that the excipient increases the cellular uptake, specific or non-specific organelle uptake, stability, solubility, half-life, binding efficacy, specificity, targeting, distribution, absorption, or renal clearance of the inactive or active compound. Alternatively, or in addition, the pharmaceutically acceptable carrier increases or decreases the immunogenicity of the inactive or active compound.

Alternatively, or in addition, pharmaceutically acceptable carriers are salts (for example, acid addition salts, e.g., salts of hydrochloric, hydrobromic, acetic acid, and benzene sulfonic acid), esters, salts of such esters, or any other compound which, upon administration to a subject, are capable of providing (directly or indirectly) the inactive or active compounds of the invention. Pharmaceutically acceptable carriers are alternatively or additionally diluents, excipients, adjuvants, emulsifiers, buffers, stabilizers, and/or preservatives.

Pharmaceutically acceptable carriers of the invention include delivery systems/mechanisms that increase uptake of the inactive compound by targeted cells. For example, pharmaceutically acceptable carriers of the invention are viruses, recombinant viruses, engineered viruses, viral particles, replication-deficient viruses, liposomes, cationic lipids, cationic small molecules, anionic lipids, anionic small molecules, cationic polymers, delocalized lipophilic cations, delocalized lipophilic anions, polymers, hydrogels, micro- or nano-capsules (biodegradable), micropheres (optionally bioadhesive), cyclodextrins, plasmids, mammalian expression vectors, proteinaceous vectors, any type of cell, or any combination of the preceding elements (see, O'Hare and Normand, International PCT Publication No. WO 00/53722; U.S. Patent Publication 2008/0076701). Moreover, pharmaceutically acceptable carriers that increase cellular uptake can be modified with cell-specific proteins or other elements such as receptors, ligands, antibodies to specifically target cellular uptake to a chosen cell type. These molecules, oligomers, polymers, and others as mentioned prior can specifically designed for different types of cellular uptake. For example, they can be engineered to increase or decrease use of phagocytosis, passive diffusion, facilitated diffusion, osmosis, passing through channels, aquaporins, membranes, using carrier proteins, active transport, moving with concentration gradients, filtration, membrane potentials, and different pressures.

In another aspect of the invention, compositions are first introduced into a cell or cell population that is subsequently administered to a subject. In some embodiments, the inactive compound is delivered intracellularly, e.g., in cells of a target tissue such as lung, or in inflamed tissues. Included within the invention are compositions and methods for delivery of the inactive compound and/or composition by removing cells of a subject, delivering the isolated inactive compound or composition to the removed cells, and reintroducing the cells into a subject. In some embodiments, a miRNA and/or miRNA inhibitor molecule is combined with a cationic lipid or transfection material such as LIPOFECTAMINE (Invitrogen).

In one aspect, the active compounds are prepared with pharmaceutically acceptable carriers that will protect inactive or active compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Examples of materials which can form hydrogels include polylactic acid, polyglycolic acid, PLGA polymers, alginates and alginate derivatives, gelatin, collagen, agarose, natural and synthetic polysaccharides, polyamino acids such as polypeptides particularly poly(lysine), polyesters such as polyhydroxybutyrate and poly-epsilon.-caprolactone, polyanhydrides; polyphosphazines, poly(vinyl alcohols), poly(alkylene oxides) particularly poly(ethylene oxides), poly(allylamines) (PAM), poly(acrylates), modified styrene polymers such as poly(4-aminomethylstyrene), pluronic polyols, polyoxamers, poly(uronic acids), poly(vinylpyrrolidone), polydienol ethers, polydienamines, polydivinyl thioethers, polyvinyl ethers, and copolymers of the above, including graft copolymers.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Pharmaceutically acceptable carriers are cationic lipids that are bound or associated with miRNA and/or miRNA inhibitor. Alternatively, or in addition, the inactive compounds are encapsulated or surrounded in cationic lipids, e.g. lipsosomes, for in vivo delivery. Exemplary cationic lipids include, but are not limited to, N41-(2,3-dioleoyloxy)propyliN,N,N-trimethylammonium chloride (DOTMA); 1,2-bis(oleoyloxy)-3-3-(trimethylammonium)propane (DOTAP), triphenylphosnium compounds (TPP), 1,2-bis(dimyrstoyloxy)-3-3-(trimethylammonia)propane (DMTAP); 1,2-dimyristyloxypropyl-3-dimethylhydroxyethylammonium bromide (DMRIE); dimethyldioctadecylammonium bromide (DDAB); 3-(N—(N′,N′-dimethylaminoethane)carbamoyl)cholesterol (DC-Chol); 3β-[N′,N′-diguanidinoethyl-aminoethane)carbamoyl cholesterol (BGTC); 2-(2-(3-(bis(3-aminopropyl)amino)propylamino)acetamido)-N,N-ditetradecyla-cetamide (RPR209120); pharmaceutically acceptable salts thereof, and mixtures thereof. Further exemplary cationic lipids include, but are not limited to, 1,2-dialkenoyl-sn-glycero-3-ethylphosphocholines (EPCs), such as 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, pharmaceutically acceptable salts thereof, and mixtures thereof.

Exemplary polycationic lipids include, but are not limited to, tetramethyltetrapalmitoyl spermine (TMTPS), tetramethyltetraoleyl spermine (TMTOS), tetramethlytetralauryl spermine (TMTLS), tetramethyltetramyristyl spermine (TMTMS), tetramethyldioleyl spermine (TMDOS), pharmaceutically acceptable salts thereof, and mixtures thereof. Further exemplary polycationic lipids include, but are not limited to, 2,5-bis(3-aminopropylamino)-N-(2-(dioctadecylamino)-2-oxoethyl)pentanamide (DOGS); 2,5-bis(3-aminopropylamino)-N-(2-(di(Z)-octadeca-9-dienylamino)-2-oxoethyl)pentanamide (DOGS-9-en); 2,5-bis(3-aminopropylamino)-N-(2-(di(9Z,12Z)-octadeca-9,12-dienylamino)-2-oxoethyl)pentanamide (DLinGS); 3-beta-(N4-(N1, N8-dicarbobenzoxyspermidine)carbamoyl)cholesterol (GL-67); (9Z,9yZ)-2-(2,5-bis(3-amninopropylamino)pentanamido)propane-1,3-diyl-dioct-adec-9-enoate (DOSPER); 2,3-dioleyloxy-N-[2(sperminecarboxamido)ethyl]-N,N-dimethyl-1-propanamini-urn trifluoro-acetate (DOSPA); pharmaceutically acceptable salts thereof, and mixtures thereof.

Examples of cationic lipids are described in U.S. Pat. Nos. 4,897,355; 5,279,833; 6,733,777; 6,376,248; 5,736,392; 5,334,761; 5,459,127; 2005/0064595; U.S. Pat. Nos. 5,208,036; 5,264,618; 5,279,833; 5,283,185; 5,753,613; and 5,785,992; each of which is incorporated herein in its entirety.

Pharmaceutically acceptable carriers of the invention also include non-cationic lipids, such as neutral, zwitterionic, and anionic lipids. Exemplary non-cationic lipids include, but are not limited to, 1,2-Dilauroyl-sn-glycerol (DLG); 1,2-Dimyristoyl-snglycerol (DMG); 1,2-Dipalmitoyl-sn-glycerol (DPG); 1,2-Distearoyl-sn-glycerol (DSG); 1,2-Dilauroyl-sn-glycero-3-phosphatidic acid (sodium salt; DLPA); 1,2-Dimyristoyl-snglycero-3-phosphatidic acid (sodium salt; DMPA); 1,2-Dipalmitoyl-sn-glycero-3-phosphatidic acid (sodium salt; DPPA); 1,2-Distearoyl-sn-glycero-3-phosphatidic acid (sodium salt; DSPA); 1,2-Diarachidoyl-sn-glycero-3-phosphocholine (DAPC); 1,2-Dilauroyl-sn-glycero-3-phosphocholine (DLPC); 1,2-Dimyristoyl-sn-glycero-3-phosphocholine (DMPC); 1,2-Dipalmitoyl-sn-glycero-0-ethyl-3-phosphocholine (chloride or triflate; DPePC); 1,2-Dipalmitoyl-sn-glycero-3-phosphocholine (DPPC); 1,2-Distearoyl-sn-glycero-3-phosphocholine (DSPC); 1,2-Dilauroyl-sn-glycero-3-phosphoethanolamine (DLPE); 1,2-Dimyristoyl-sn-glycero-3-phosphoethanolamine (DMPE); 1,2-Dipalmitoyl-sn-glycero-3-phosphoethanolamine (DPPE); 1,2-Distearoylsn-glycero-3-phosphoethanolamine (DSPE); 1,2-Dilauroyl-sn-glycero-3-phosphoglycerol (sodium salt; DLPG); 1,2-Dimyristoyl-sn-glycero-3-phosphoglycerol (sodium salt; DMPG); 1,2-Dimyristoyl-sn-glycero-3-phospho-sn-1-glycerol (ammonium salt; DMP-sn1-G); 1,2-Dipalmitoyl-sn-glycero-3-phosphoglycerol (sodium salt; DPPG); 1,2-Distearoyl-sn-glycero-3-phosphoglycero (sodium salt; DSPG); 1,2-Distearoyl-snglycero-3-phospho-sn-1-glycerol (sodium salt; DSP-sn-1-G); 1,2-Dipalmitoyl-snglycero-3-phospho-L-serine (sodium salt; DPP S); 1-Palmitoyl-2-linoleoyl-sn-glycero-3-phosphocholine (PLinoPC); 1-Palmitoyl-2-oleoyl-sn-glycero-3-phosphocholine (POPC); 1-Palmitoyl-2-oleoyl-sn-glycero-3-phosphoglycerol (sodium salt; POPG); 1-Palmitoyl-2-oleoyl-sn-glycero-3-phosphoglycerol (sodium salt; POPG); 1-Palmitoyl-2-oleoyl-snglycero-3-phosphoglycerol (ammonium salt; POPG); 1-Palmitoyl-2-4-o-sn-glycero-3-phosphocholine (P-lyso-PC); 1-Stearoyl-2-lyso-sn-glycero-3-phosphocholine (S-lysoPC); and mixtures thereof. Further exemplary non-cationic lipids include, but are not limited to, polymeric compounds and polymer-lipid conjugates or polymeric lipids, such as pegylated lipids, including polyethyleneglycols, N-(Carbonylmethoxypolyethyleneglycol-2000)-1,2-dimyristoyl-sn-glycero-3-phosphoethanolamine (sodium salt; DMPE-MPEG-2000); N-(Carbonyl-methoxypolyethyleneglycol-5000)-1,2-dimyristoyl-sn-glycero-3-phosphoethanolamine (sodium salt; DMPE-MPEG-5000); N(Carbonyl-methoxypolyethyleneglycol 2000)-1,2-dipalmitoyl-sn-glycero-3-phosphoethanolamine (sodium salt; DPPE-MPEG-2000); N-(Carbonyl-methoxypolyethyleneglycol 5000)-1,2-dipalmitoyl-sn-glycero-3-phosphoethanolamine (sodium salt; DPPE-MPEG-5000); N-(Carbonyl-methoxypolyethyleneglycol 750)-1,2-distearoyl-sn-glycero-3-phosphoethanolamine (sodium salt; DSPE-MPEG-750); N(Carbonyl-methoxypolyethyleneglycol 2000)-1,2-distearoyl-sn-glycero-3-phosphoethanolamine (sodium salt; DSPE-MPEG-2000); N-(Carbonylmethoxypolyethyleneglycol 5000)-1,2-distearoyl-sn-glycero-3-phosphoethanolamine (sodium salt; DSPE-MPEG-5000); sodium cholesteryl sulfate (SCS); pharmaceutically acceptable salts thereof, and mixtures thereof. Examples of non-cationic lipids include, but are not limited to, dioleoylphosphatidylethanolamine (DOPE), diphytanoylphosphatidylethanolamine (DPhPE), 1,2-Dioleoyl-sn-Glycero-3-Phosphocholine (DOPC), 1,2-Diphytanoyl-sn-Glycero-3-Phosphocholine (DPhPC), cholesterol, and mixtures thereof.

Pharmaceutically-acceptable carriers of the invention further include anionic lipids. Exemplary anionic lipids include, but are not limited to, phosphatidylserine, phosphatidic acid, phosphatidylcholine, platelet-activation factor (PAF), phosphatidylethanolamine, phosphatidyl-DL-glycerol, phosphatidylinositol, phosphatidylinositol (pi(4)p, pi(4,5)p2), cardiolipin (sodium salt), lysophosphatides, hydrogenated phospholipids, sphingoplipids, gangliosides, phytosphingosine, sphinganines, pharmaceutically acceptable salts thereof, and mixtures thereof.

Supplemental or complementary methods for delivery of nucleic acid molecules for use herein are described, e.g., in Akhtar, et al., Trends Cell Bio. 2:139, 1992; Delivery Strategies for Antisense Oligonucleotide Therapeutics, ed. Akhtar, 1995; Maurer, et al., Mol. Membr. Biol. 16:129-140, 1999; Hofland and Huang, Handb. Exp. Pharmacol. 137:165-192, 1999; and Lee, et al., ACS Symp. Ser. 752:184-192, 2000. Sullivan, et al., International PCT Publication No. WO 94/02595, further describes general methods for delivery of enzymatic nucleic acid molecules. These protocols can be utilized to supplement or complement delivery of virtually any inactive compound of the invention.

Activation of the pharmaceutical is most rapid where the inactive compound is exposed to a relatively high concentration of ROS, another reactive species or free radicals, depending on the compound. Thus, while the compounds of the present invention could be formulated to be administered systemically, pharmaceutical treatments that, e.g., provide for exposure of the active compound to the air (where an ROS reaction creates the active agent) or inflammatory tissues (where a free radical reaction or ROS reaction creates the active agent) can provide effective release of the active compound over time, such that administration of the inactive compound to provide a steady dosage of the active compound can be less frequent than the administration of the active compound.

In various embodiments, the pharmaceutical is useful for treatment of a lung, eye, skin, nasal, oral, scalp, hair or nail disease or disorder. Such tissues are expected to be exposed to a significant about of ROS, from, e.g., ozone in the air. The pharmaceutical can also target particular cells (e.g., tumor cells) or subcellular organelles (e.g., mitochondria) that produce free radicals, ROS or other reactive species.

Inflammatory tissues are also a target of the above compounds, since those tissues have free radicals, ROS, and other reactive species. For example, hydroxyl radicals, superoxide, and hydrogen peroxide are abundant in cells experiencing oxidative stress, for example cancerous and inflammatory tissues, neurological diseases, autoimmune diseases, cardiovascular diseases, arthritis, etc. (Finosh et al., 2013).

Any pharmaceutical that can be activated from the invention compounds are within the scope of the current invention. In certain embodiments, the pharmaceutical is an oligopeptide, a polypeptide, or a steroid, for example estrone, cortisol, corticosterone, aldosterone, progesterone, testosterone, or dihydrotestosterone.

The pharmaceutical can also be a nutrient, e.g., vitamin D, or any other nutrient.

Nonlimiting examples of pharmaceuticals that can be activated from the invention compounds include the pharmaceuticals listed on pp. 16-47 of U.S. Provisional Patent Application 62/428,137, incorporated by reference.

In some of these embodiments, the pharmaceutical is gemcitabine. In some of those embodiments, the compound is LXXXVII, LXXXVIII, LXXXIX, or XC.

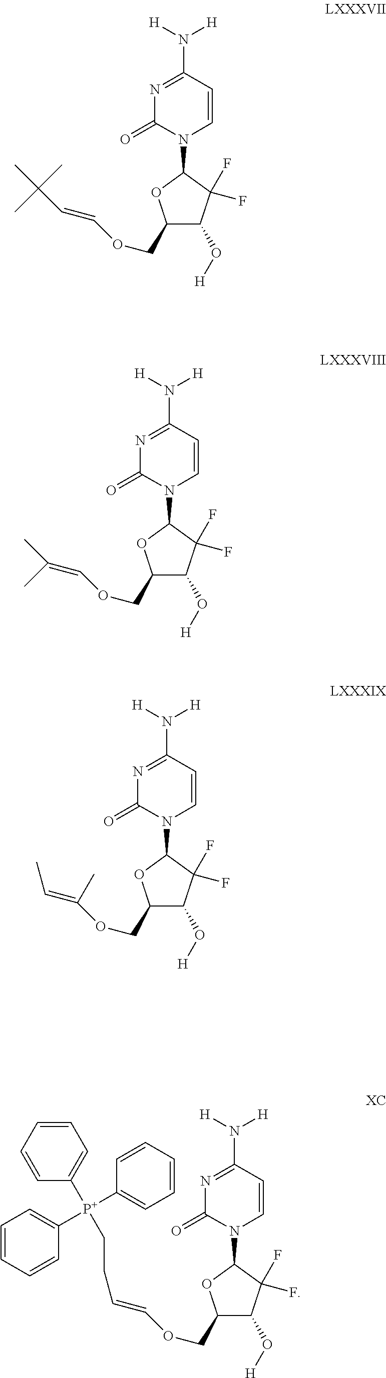 [SMILES: CC(C)(C)/C=C/OC[C@H]1O[C@@H](n2ccc(N)nc2=O)C(F)(F)[C@@H]1O ; CC(C)=COC[C@H]1O[C@@H](n2ccc(N)nc2=O)C(F)(F)[C@@H]1O ; C/C=C(\C)OC[C@H]1O[C@@H](n2ccc(N)nc2=O)C(F)(F)[C@@H]1O ; Nc1ccn([C@@H]2O[C@H](CO/C=C/CC[P+](c3ccccc3)(c3ccccc3)c3ccccc3)[C@@H](O)C2(F)F)c(=O)n1]

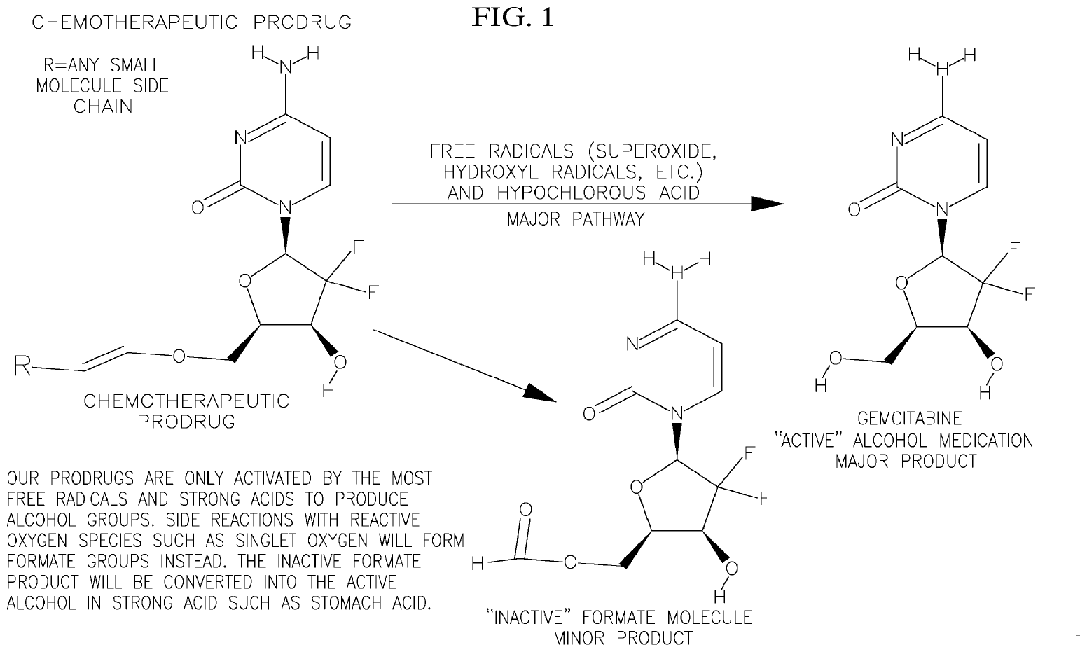

FIG. 1 shows a generalized mechanism for the above four gemcitabine prodrugs. As shown therein, gemcitabine is an alcohol, and the hydroxyl group is formed from the enol ether in the above prodrugs. These compounds illustrate considerations that are made when designing prodrugs encompassing the present invention. All of these compounds form gemcitabine upon exposure to a free radical. However, compounds LXXXVII and LXXXVIII produces gemcitabine and an aldehyde, whereas LXXXIX produces gemcitabine and a ketone, and would likely have a different reaction rate than the reactions with LXXXVII and LXXXVIII. Compound XC is more hydrophobic and has a positive charge, so designed to target the mitochondrial matrix.

In other embodiments, the pharmaceutical is cytarabine. An example of a cytarabine prodrug invention compound is XCI.

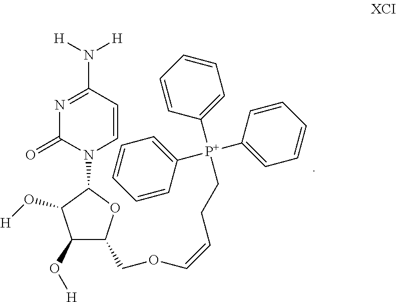 [SMILES: Nc1ccn([C@@H]2O[C@H](CO/C=C\CC[P+](c3ccccc3)(c3ccccc3)c3ccccc3)[C@@H](O)[C@@H]2O)c(=O)n1]

Similar to compound XC above, compound XCI is hydrophobic with a positive charge and would be expected to target the mitochondrial matrix.

In further embodiments, the pharmaceutical is camptothecin. An example of a prodrug of camptothecin is XCII

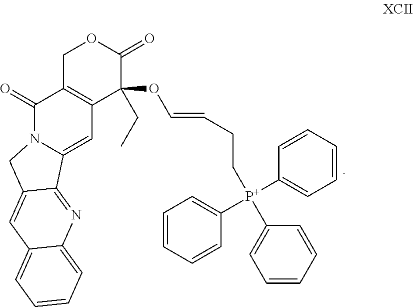 [SMILES: CC[C@@]1(O/C=C/CC[P+](c2ccccc2)(c2ccccc2)c2ccccc2)C(=O)OCc2c1cc1n(c2=O)Cc2cc3ccccc3nc2-1]

Prodrugs of camptothecin, including topotecan, irinotecan, silatecan, cositecan, exatecan, lurtotecan, gimatecan, belotecan, and rubitecan, can be prepared using the guidance provided herein.

In additional embodiments, the pharmaceutical is a nutrient, an antibiotic, an antifungal, an antiviral or an antiparasitic, as described above.

In some of these embodiments, the pharmaceutical is zalcitabine, a reverse transcriptase inhibitor (NARTI) that is used to treat HIV/AIDS. An example of such a compound is XCIII

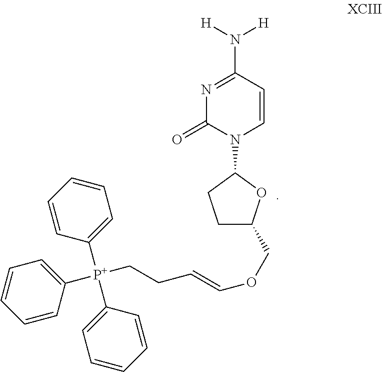 [SMILES: Nc1ccn([C@H]2CC[C@@H](CO/C=C/CC[P+](c3ccccc3)(c3ccccc3)c3ccccc3)O2)c(=O)n1]

A prodrug of lamivudine, another antiviral compound, can also be prepared. Examples of such a compound are XCIV and XCV.

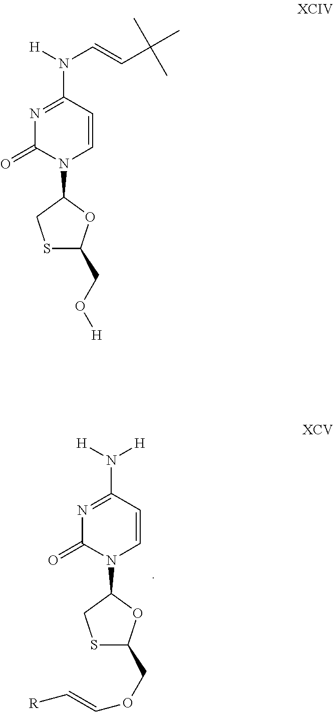

XCIV has an enamine rather than an enol ether for reaction with a free radical.

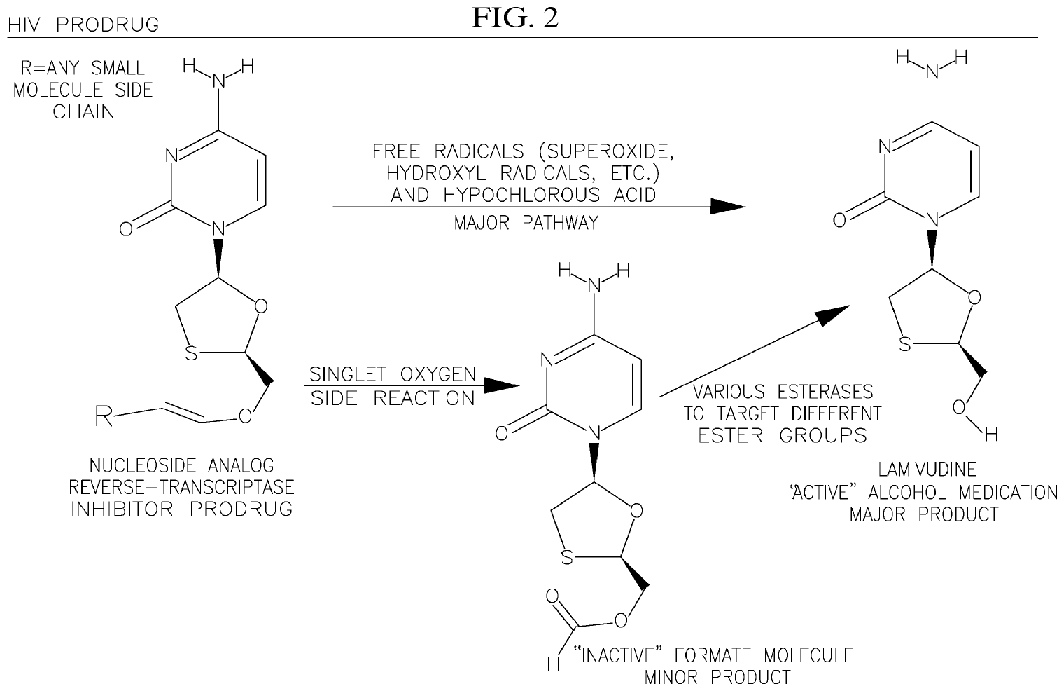

XCV is designed to form the active compound lamivudine in a mammalian body whether the compound reacts with a free radical or an ROS. See FIG. 2. When XCV reacts with an ROS, the formate derivative of lamivudine is produced. That derivative is subject to reaction with an esterase to convert the formate derivative into lamicudine. Since esterases are ubiquitous in the mammalian (including human) body, administration of the above invention compounds that (a) react with a free radical to form the active agent and (b) react with an ROS to form the aldehyde or formate of the active agent, will likely all become the active agent, since the ubiquitous esterases would convert the aldehyde or formate into the active agent. This also applies to enamines, which react with ROS to form an amide bond, and vinyl thioethers, which react with ROS to form a thioester bond, since amides and thioesters are both subject to esterase action to form an amine or thiol.

Compounds that produce an active agent upon reaction with free radicals or ROS can be configured so that the active agent is dimerized such that two active agents are created upon reaction with a free radical or ROS. Such compounds require half as many free radical or ROS reactions to produce an equivalent amount of active agent. Examples are provided in compounds XCVI and XCVII below.

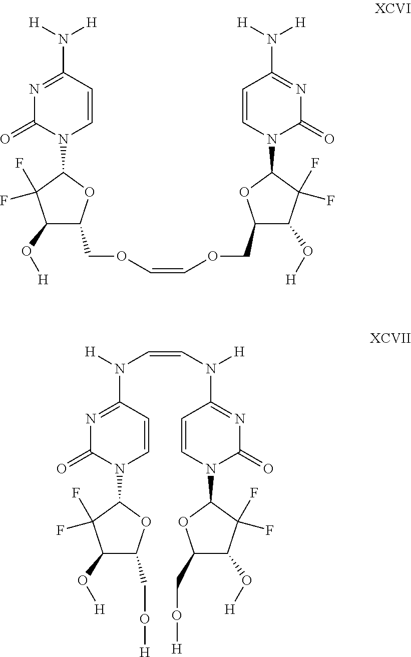 [SMILES: Nc1ccn([C@@H]2O[C@H](CO/C=C\OC[C@H]3O[C@@H](n4ccc(N)nc4=O)C(F)(F)[C@@H]3O)[C@@H](O)C2(F)F)c(=O)n1 ; O=c1nc(N/C=C\Nc2ccn([C@@H]3O[C@H](CO)[C@@H](O)C3(F)F)c(=O)n2)ccn1[C@@H]1O[C@H](CO)[C@@H](O)C1(F)F]

Compound XCVI produces two gemcitabine molecules on reaction with a free radical, hydrogen peroxide or superoxide, through hydrolysis of two joined enol ether bonds. Compound XCVII also produces two gemcitabine molecules on reaction with a free radical, but through two joined enamine bonds.

With all of the prodrug compounds LXXXVII-XCIV, XCVI, XCVII and XCV above, the active pharmaceutical is only formed upon reaction with free radicals, ROS or another reactive species. Prodrug and other invention compounds can be similarly designed to be active upon reaction with free radicals, ROS or another reactive species. Examples are provided in PCT/US2016/052529 and WO/2016/023015, using ozone as an exemplary reactant.

In some embodiments, the invention compound is designed to simply react with a free radical, ROS or another reactive species to prevent deleterious reactions, i.e., a free radical or ROS scavenger. In those embodiments, it is often preferred that, upon reaction with a free radical, ROS or another reactive species, a non-toxic, naturally occurring product, or a non-toxic derivative thereof, is created from the compound. As previously discussed, these agents can not only prevent harmful free radical, oxidation and reactive species reactions, but control the reaction to produce specific molecules that are non-toxic and/or useful.

The compounds of the present invention can be used systemically to generally react with free radicals, ROS or another reactive species throughout a biological system, e.g., a mammal, such as a human. Such a use is akin to taking an antioxidant such as ascorbic acid.

The invention compounds can also be used in a biological system to reduce biologically important free radicals, ROS or other reactive species. An example is nitric oxide (NO). NO is a free radical that is important in neuroscience, physiology and immunity as a signaling molecule. As such, increasing NO signaling by administration of NO or drugs that increase NO production or responsiveness are the basis of current treatments and treatments under development for hypertension, heart failure, ischemia, stroke, erectile dysfunction and other conditions (Forte et al., 2016). However, excess NO exacerbates inflammatory bowel diseases (IBD) (Soufli et al., 2016), and NO administration can cause dangerous side effects such as low blood pressure. The invention compounds can thus be administered to reduce excess NO in IBD and when NO administration causes side effects or is overdosed.

The compounds described herein can be applied to food technology. In that regard, any coating, antioxidant, preservative, flavor component, antibacterial, antifungal, or any other compound used in food preparation can be incorporated into compounds of the present invention that are monomers, oligomers or polymers to provide a slow release compound that maintains its useful characteristic on or in the food or food packaging for a longer time. Such compounds are useful for meat, breads, fruit, vegetables, cheeses, oils, or any other food, and are particularly useful for foods that can undergo oxidative or free radical reactions. Additionally, compounds that produce indicators or volatile compounds when oxidative or free radical reactions occur, or when harmful organisms or toxins such as Salmonella spp., botulism, etc. are present e.g., a fragrance, smell, color change, fluorescent change, etc.

In foods, ROS, other reactive species and free radicals produce a wide range of different byproducts when they target unsaturated fatty acids. Over time, foul-smelling ketones and aldehydes are produced in the reaction, many of which are toxic and can be bad for our health. As a result, antioxidants are incorporated into foods and beverages to help control and prevent these unwanted chemical reactions from taking place. However, creating natural antioxidants synthetically in a lab is a rather expensive process as a result of their complex structures. As a result, new types of antioxidants were created such as butylated hydroxyanisole, butylated hydroxytoluene, propyl gallate, and tertiary butyl hydroquinone. However, these synthetic antioxidants have had much scrutiny, and when incorporating them into food some of them are only allowed to have a maximum certain concentration as a result of possible suspected carcinogenicity. As a result, additional synthetic antioxidants and free radical scavengers are needed that can be produced on a mass scale very inexpensively, while controlling the byproducts created from these chemical reactions in a safe, and potentially beneficial manner.

The compounds of the present invention that can be used as food and beverage antioxidants and free radical scavengers can be designed to produce pure flavors, ingredients, and even essential amino acids upon free radical or ROS reaction.

In some embodiments, the antioxidant and free radical scavenger useful for food preservation produces vanillin on reaction with a free radical or ROS. Examples of such compounds are XCVIII and XCIX.

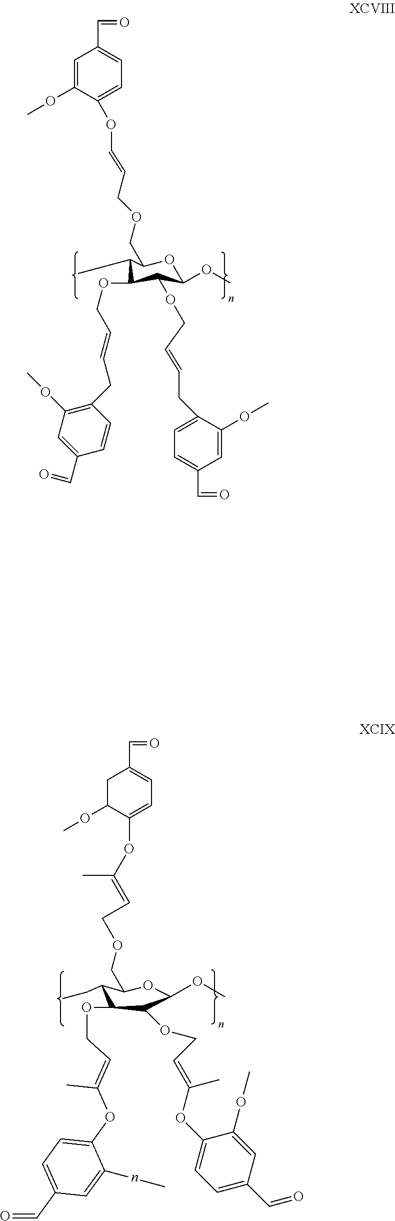

While XCVIII and XCIX produce vanillin upon reaction with a free radical, they produce vanillin formate (XCVIII) or vanillin acetate (XCIX) upon reaction with specific ROS such as ozone or singlet oxygen. However, the ester in vanillin formate hydrolyzes to produce the pure vanillin molecule when exposed to a strong acid such as stomach acid, or through enzyme reactions, and other metabolic means. After free radical or ROS reaction, the remaining polymer is a cellulosic fiber. These two compounds differ in that the enol ether reaction of XCVIII produces an aldehyde on the polymeric product, while XCIX produces a ketone polymeric product. As such, the speed of the reaction with XCVIII should differ from the speed of the reaction with XCIX.

In other embodiments, the antioxidant and free radical scavenger useful for food preservation produces asparagine on reaction with a free radical or ROS. Examples of such compounds are C and CI.

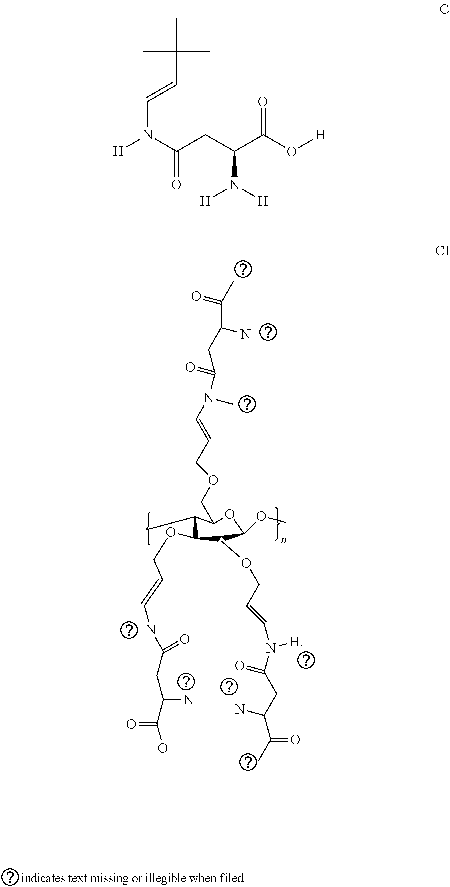

Production of asparagine from reaction of C or CI with a free radical is through an enamine bond. C is a monomer and CI is a polymer that leaves a cellulosic fiber after the reaction, like XCVIII above. Upon reaction with an ROS, the resulting formate or ketone group would hydrolyze in the stomach acid to produce asparagine.

In additional embodiments, antioxidant and free radical scavenger useful for food preservation produces cysteine on reaction with a free radical or ROS. Examples of such compounds are CII and CIII.

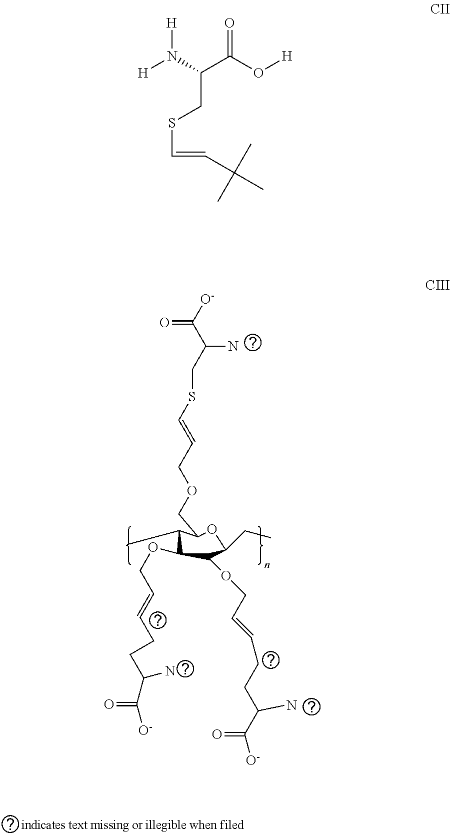

Production of cysteine from reaction of CII or CIII with a free radical is through a vinyl thioether bond. CII is a monomer and CIII is a polymer that leaves a cellulosic fiber after the reaction, like XCVIII above. Upon reaction with an ROS, the resulting formate or ketone group would hydrolyze in the stomach acid to produce cysteine.

In other embodiments, antioxidant and free radical scavenger useful for food preservation produces serine on reaction with a free radical or ROS. Examples of such compounds are CIV and CV.

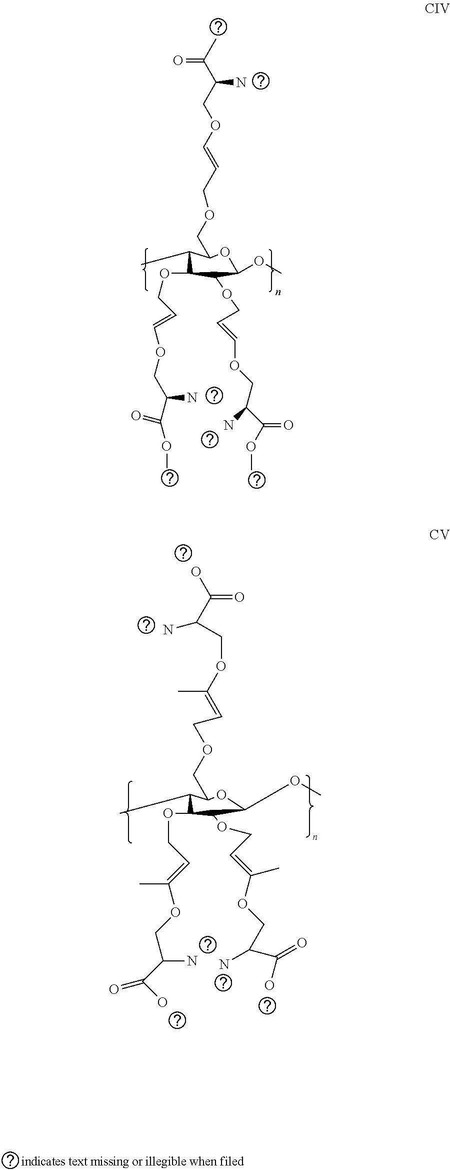

These two compounds differ in that the enol ether reaction of CIV produces an aldehyde on the polymeric product, while CV produces a ketone polymeric product. As such, the speed of the reaction with CV should differ from the speed of the reaction with CIV.

The compounds of the present invention can also be used in formulations for skin care, where the ability of the compounds to scavenge free radicals and ROS protect the skin from damage. As such, the compounds are also useful in sunblocks where they prevent reactivity of free radicals caused by UV light from the sun. The compounds can also be formulated to produce a beneficial product such as vitamin D or an exfoliant. Thus, these invention compounds are also useful in cosmetics, deodorants, fragrances, nails, hair, eye lashes, make-up, perfumes, etc. and can be formulated to be included in absorbents, anti-acne, antioxidants, cleansing agents, coloring agents/pigments, emollients, emulsifiers, exfoliants, film-forming/holding agents, fragrances, hydration, plant extracts, preservatives, scrubbing agents, sensitizing agents, silicones, skin-replenishing, skin-restoring, skin-softening, skin-soothing, slip agents, sunscreen actives, texture enhancers, thickeners, emulsifiers, and vitamins.

In various embodiments, the invention compound produces vitamin D upon reaction with a free radical, ROS or another reactive species. Examples of such compounds are CVI and CVII.

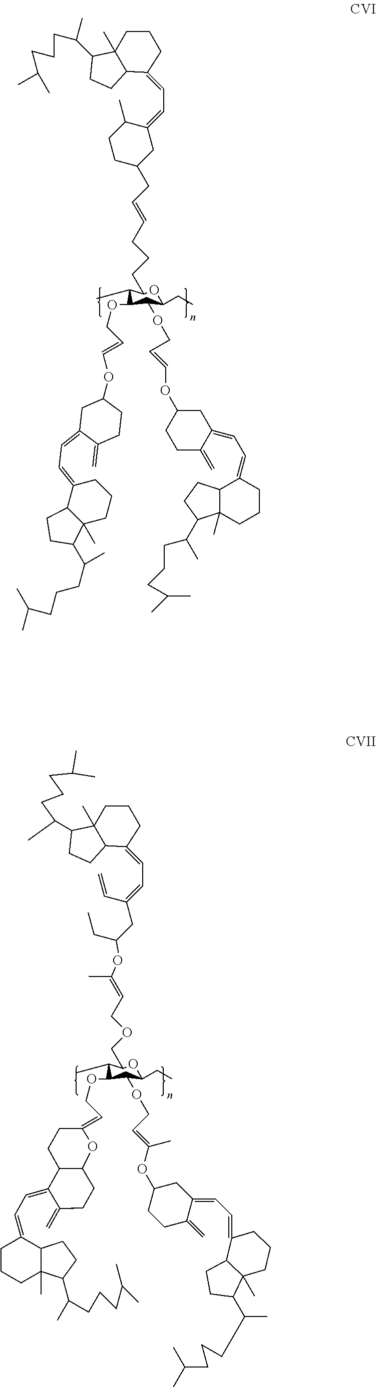

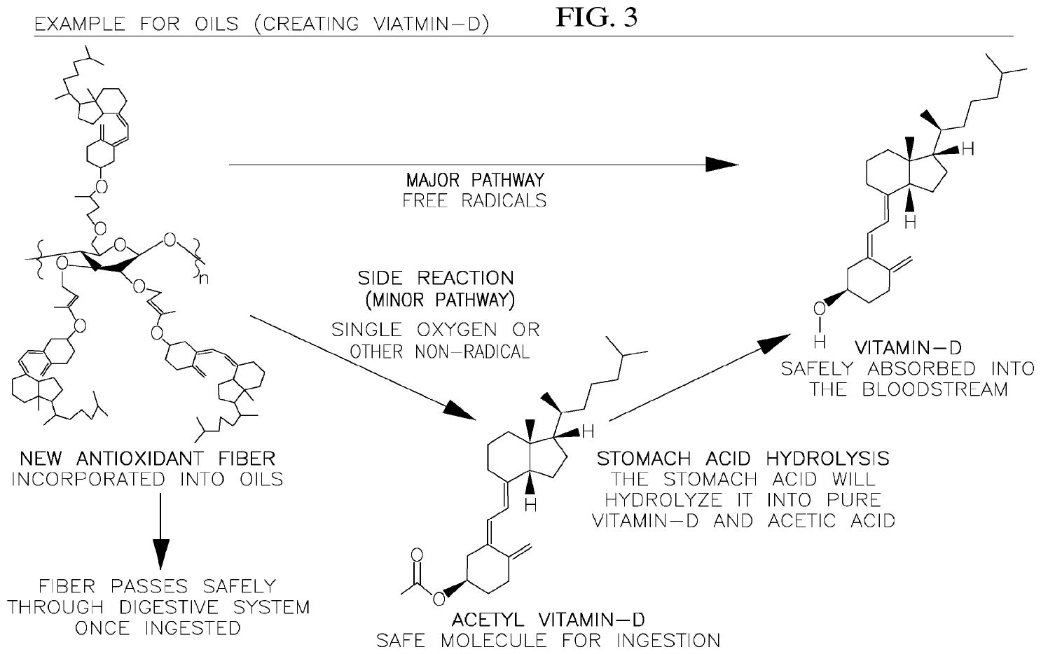

These compounds can also be used in foods, where they prevent reaction of food ingredients with free radicals or ROS. For example they can be incorporated into oils to prevent oil oxidation. See FIG. 3.

Compound CVI differs from CVII in that the enol ether reaction of CVI produces an aldehyde on the polymeric product, while CVII produces a ketone polymeric product. As such, the speed of the reaction with CVI should differ from the speed of the reaction with CVII.

In additional embodiments, the invention compound produces glycolate, an exfoliant, upon reaction with a free radical or ROS. An example of such a compound is CVIII

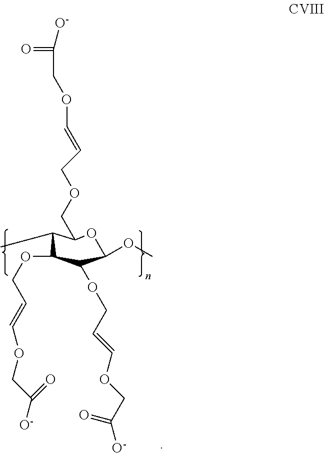

Additional compounds that react with free radicals, ROS and other reactive species include CIX, CX, CXI, CXII, CXIII, CXIX, CXX, CXXI, and CXXII

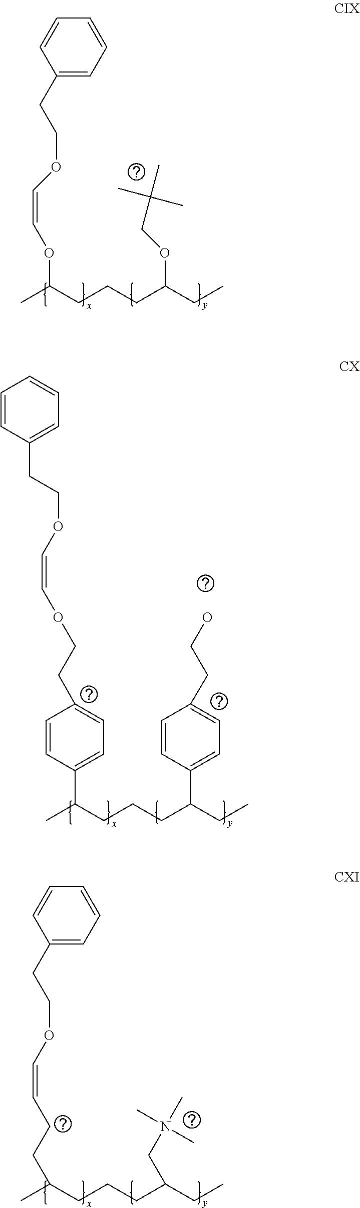

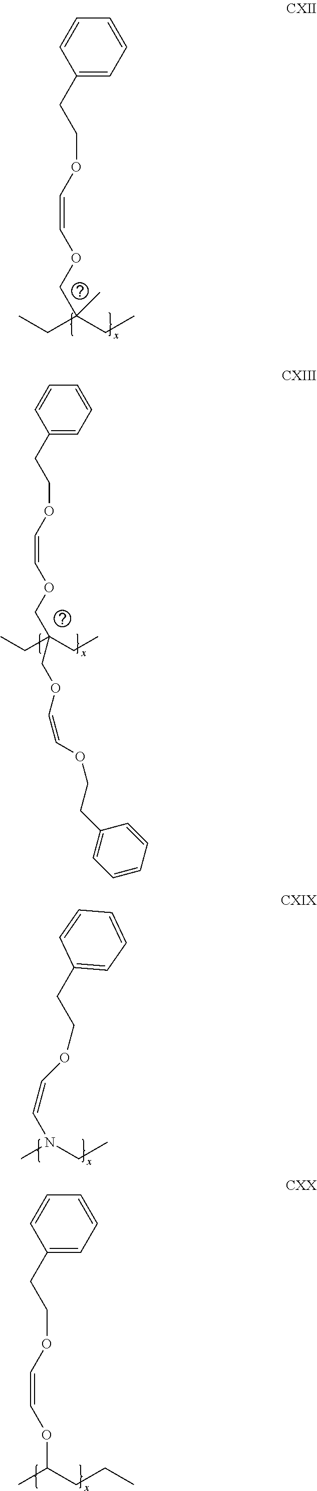

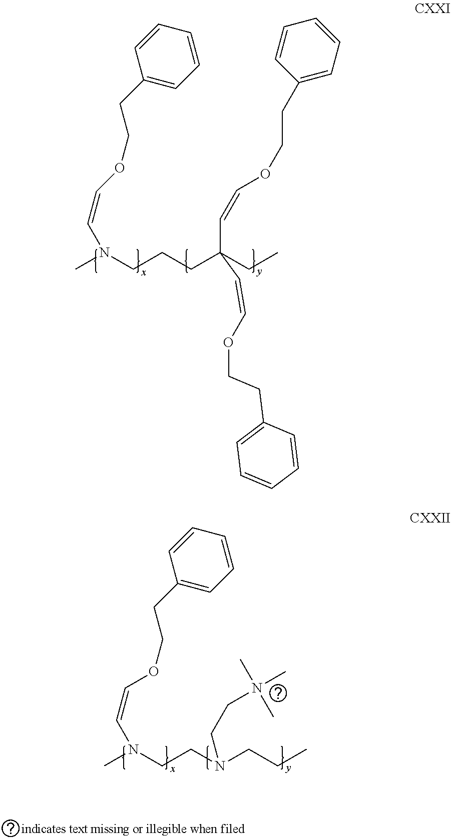

With regard to compound CXIX, it is noted that the nitrogen could be protonated at different pHs, which would make it unreactive in solution, and upon application, could then become unprotonated which will then cause it to be more reactive to ozone. This protonation is a useful tool to stabilize CXIX and similar compounds in a solution or product for long periods of time.

### Conjugated Moieties Increasing Resonance

The present invention introduces a conjugated moiety operably joined to the reacting alkene moiety allowing the use of activating agents that are not adjacent to the alkene moiety, as illustrated in Scheme 11:

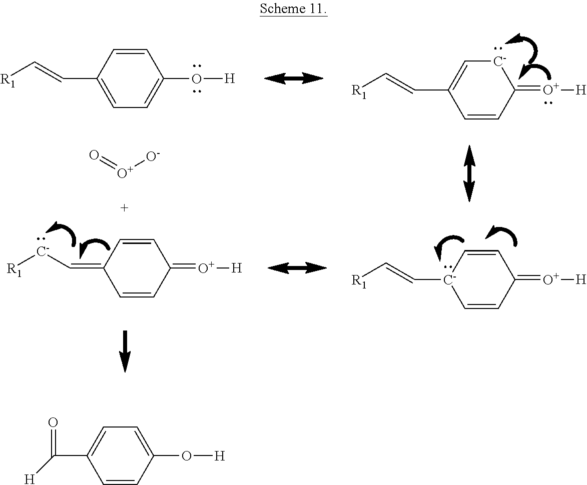

In Scheme 11, the hydroxyl moiety on the aromatic ring activates the reacting double bond through resonance through the aromatic ring. Thus, the present invention provides a similar mechanism as described above with enol ethers, enamines and vinyl thioethers, where resonance causes the formation of an oxonium ion, where an extra electron is gained when the activating group is in the ortho and para positions from the reacting double bond. At the para position, this extra electron resonates through the aromatic group, causing the formation of an extra electron on the ozone reactive double bond, which causes it to gain considerable amount more electron density, and allows it to react faster with the ROS, other reactive species, free radicals and strong acids. However, in contrast to the mechanism described above, in one of the resonance structures in the present compounds, 5 valence electrons are present on one of the carbons in the target double bond system, to give one of those two carbons a formal negative charge in one of the resonance structures. By contrast, in the enol ether, enamine and vinyl thioether embodiments, there is an ester group on one side, which prevents the 5 electrons from being gained in the resonated structure. Therefore, the present mechanism has higher reactivity towards ROS (including ozone), other reactive species, free radicals and strong acids, due to greater electron density. Resonance in conjugated structures such as the compounds of the present invention is highly predictable, as generally described in http://crab.rutgers.edu/˜alroche/Ch17.pdf.

The determination of whether any specific compound, generically provided or not provided herein, can react with a strong acid, or degrade any specific free radical, ROS or other reactive species, can be determined without undue experimentation by the skilled artisan using general knowledge and the information provided herein.

The mechanism of resonance through a conjugated moiety is advantageous over the enol ether/enamine/vinyl thioether mechanism provided above because activating groups at considerable distance from the reactive double bond can be utilized, and an enol ether, a vinyl thioether or an enamine is not required to serve as the activating agent. Also, the conjugated moiety, e.g., an aromatic ring, is not reactive to ozone because it is too stable, so even though there are several double bonds in the system, only the double bond of interest will react with the free radical, strong acid, RNS or ROS, and not the double bonds on the conjugated moiety, so the reaction is predictable, in spite of the presence of multiple double bonds. Also, there can be more than one activating agent on the conjugated moiety to further increase the reactivity.

The conjugated moiety in the compound may be cyclic or not cyclic; when cyclic, the conjugated moiety may be aromatic or not aromatic, and can also be heterocyclic or only containing carbon and hydrogen. Examples include cyclic structures that contain various elements such as oxygen, sulfur, and nitrogen incorporated into the cyclic structure, e.g., pyrrole or quinolone.

In various embodiments, the compound comprises a moiety that increases the ability of the compound to decompose ozone by increasing the electron density in the alkene moiety. This includes moieties on the conjugated group, e.g., the hydroxyl on the aromatic moiety in Scheme 4 above. This activating moiety can be the resonance-transmitting moiety, or it can be another moiety, either part of the conjugated moiety or separate from the conjugated moiety.

The activating group includes moieties that cause the reacting alkene moiety to have an extra electron as well as moieties that simply contribute to an increase in electron density in the alkene moiety. For example, alkyl groups on the ortho or para positions of an aromatic will also increase electron density to the double bond, it just will not be a full extra electron that a methoxy, amine, or hydroxyl group can donate, which ultimately give the double bond a formal negative charge in one of the resonance structures. Non-limiting examples of activating groups are hydroxyl, ether, amine, ester, amide, nitrile, halogen, alkyl, substituted alkyl, aromatic, unsaturated alkyls, nitro, substituted amines, and sulfo group.

Multiple conjugated moieties can also be present on the molecule. Those multiple conjugated moieties can be the same or different conjugated moieties.

When combined with an enol ether, an enamine or a vinylthioether structure, the conjugated moiety can further increase the reactivity of the double bond to an ROS or a free radical. For example, Compound CXXIII is an enol ether with an aromatic group having electron-donating methoxy groups. The aromatic ring with those methoxy groups provides additional reactivity to ROS and free radicals, to form R₁—OH.

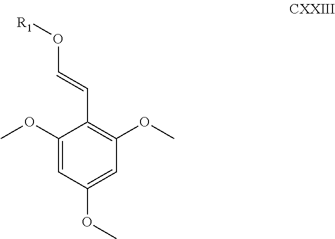

wherein R₁ is a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

Also, Compound CXXIV is an enol ether with an aromatic group having electron-donating amine groups. As with Compound CXXIII, the aromatic ring with the amine groups provides additional reactivity to ROS, another reactive species, and free radicals, to form R₁—OH.

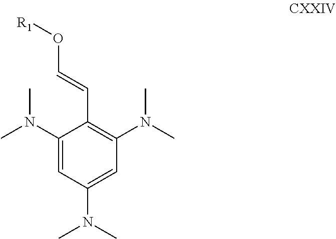

wherein R₁ is a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

Additional enol ether/enamine/vinyl thioether containing compounds that have a resonance structure are CXXV, CXXVI, or CXXVII

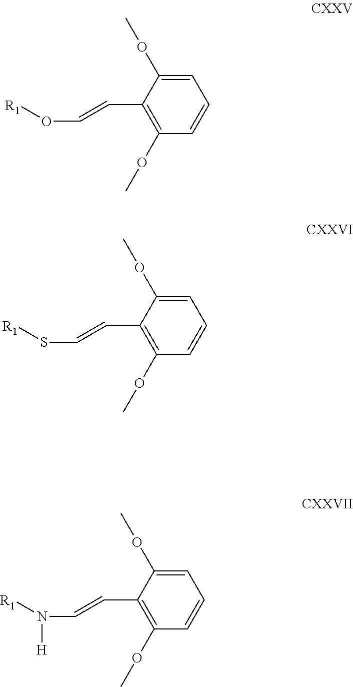

wherein R₁ a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

In some embodiments, e.g., where slower reactivity is desired, the compound comprises a moiety that decreases the reactivity of the compound by decreasing the electron density in the alkene moiety. The same moieties that increase reactivity can also decrease reactivity or have no effect when in a different position. This is illustrated through a comparison of compounds I and II below.

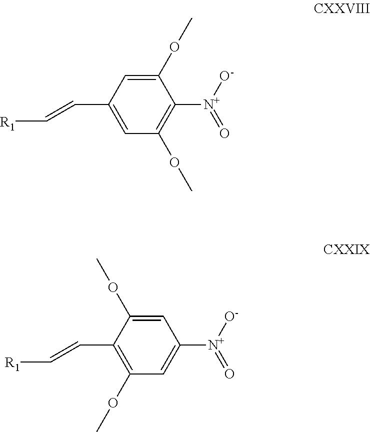

Compound Ia has two methoxy groups in the ortho position next to the nitro group. When the cation from the nitro group resonates to the ortho position having the methyl groups, those groups donate electron density to the cation. By contrast, when the cations move to both the ortho and para positions from the nitro in compound II, which has the methoxy groups in the meta position of the nitro group, the lone pair of electrons on the same ortho and para positions that have been donated from the methoxy group would stabilize the aromatic group, reducing the reactivity of the target double bond, since there is less electron density for the free radical, strong acid, ROS or RNS to react with.

Examples of additional compounds provided herewith are compounds comprising an allylic ether, an allylic ester, an anisaldehyde, and/or compound CXXX

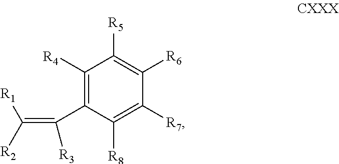

wherein R₁, R₂, R₃, R₄, R₅, R₆, R₇, and R₈ are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

Specific examples of these compounds include the cyclodextrin compound CXXXI and compounds CXXXII, CXXXIII, CXXXIV, CXXXV, CXXXVI and CXXXVII.

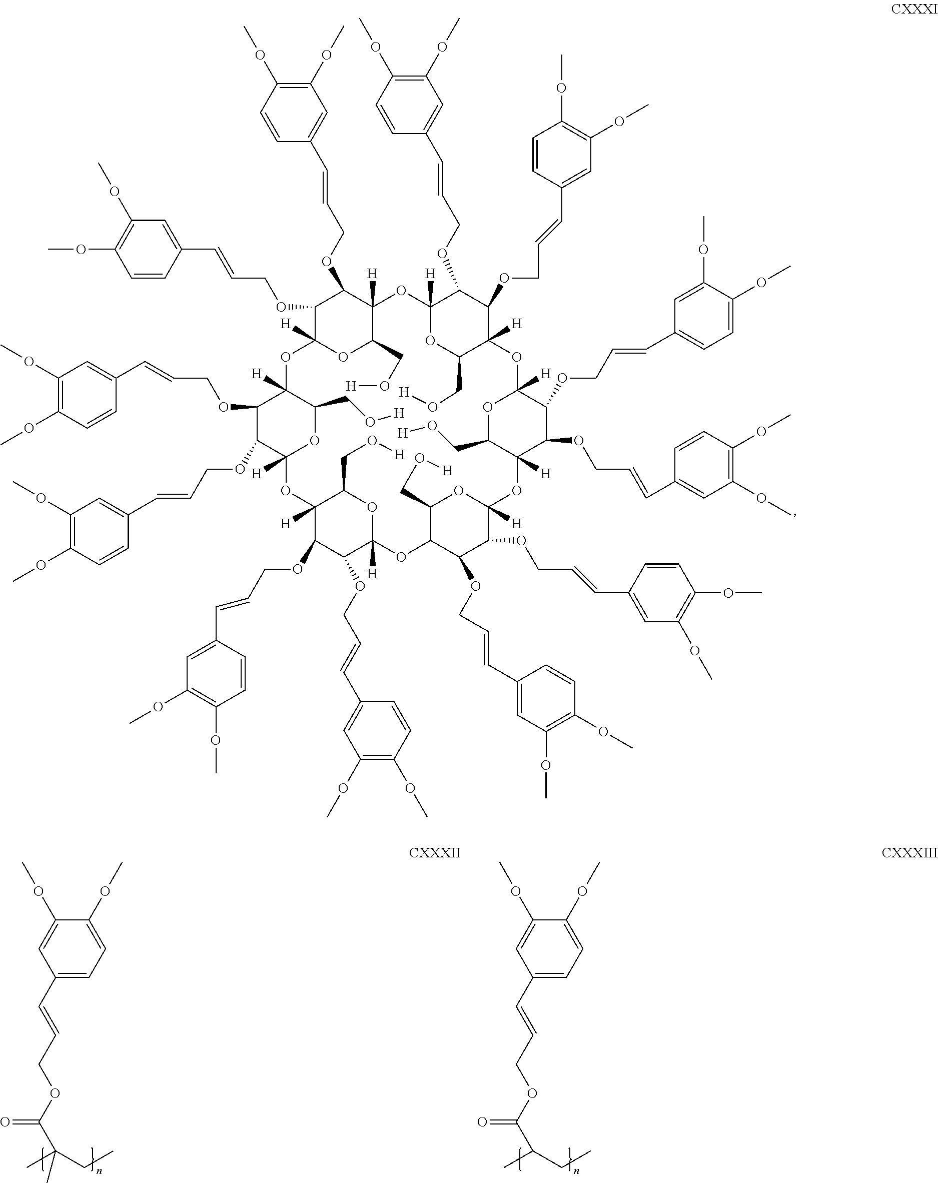

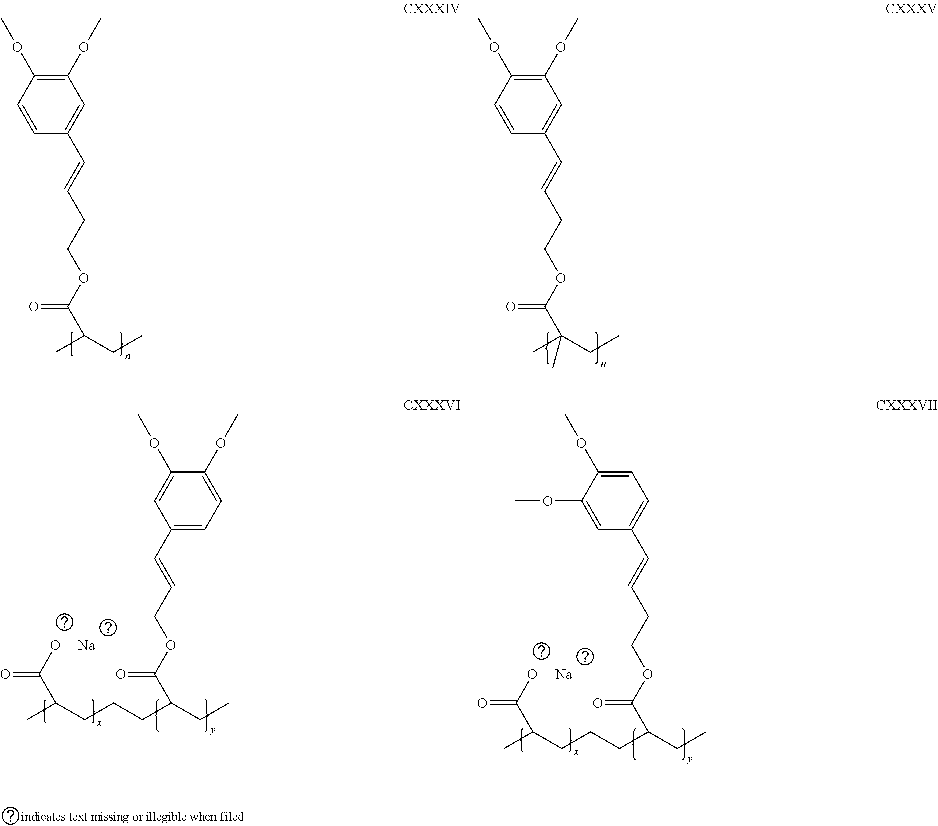

wherein n, X and Y are each independently an integer from 1 to 1,000,000.

### External Stimulation of Ozone- and ROS-Degradation

In some embodiments, the resonance-transmitting moiety or the conjugated moiety is stimulated by an external source, e.g., light or electricity, to transmit the electron through the conjugated moiety to the alkene moiety.

Thus, in additional embodiments, the present invention is directed to a compound reactive with a free radical, a reactive oxygen species (ROS) or another reactive species. The compound comprises a conjugated moiety operably joined to an alkene moiety and, optionally, a resonance-transmitting moiety. In these embodiments, the conjugated moiety or the resonance-transmitting moiety can be stimulated by an external energy source to excite an electron and/or transmit an electron through the conjugated moiety to the alkene moiety, which reacts with the free radical, strong acid, ROS or the another reactive species.

The external energy source can be any energy source that is capable of exciting an electron and/or transmit an electron through the conjugated moiety. Non-limiting examples of energy sources are heat, light, and electricity. In some of these embodiments, the external energy source is light. In other embodiments, the energy source is electricity.

Dyes and other compounds that absorb light at particular wavelengths through conjugated systems are well known in the art. When a photon of light is absorbed, an electron goes from a lower energetic molecular bonding orbital to a higher energetic anti-bonding orbital. This electron becomes part of the conduction band, and is free to be transmitted across the conjugated moiety. As a result of the electron having a higher energy, it donates more electron density to moieties subjected to the resonance of the conjugated system. By linking a free radical-, ROS—, or another reactive species-reactive alkene to the light absorbing compound, when the compound is exposed to light the increased electron density to the reactive alkene will allow the free radical, ROS or another reactive species-degrading reaction to occur much faster than without the light stimulation. As discussed above, increasing electron density at the reactive alkene increases the reaction rate of free radicals, ROS or another reactive species with the alkene, by adding an extra electron to the system, forming a formal negative charge.

These applications are not limited to the use of any particular light wavelengths, since compounds can be designed that respond to any wavelength. The activation can be with solar light, any artificial light including LEDs emitting an activating wavelength, which can be in the visible, UV or infrared bands.

Infrared light stimulation is particularly attractive, because the absorption of infrared light at less energetic wavelengths will cause molecules to vibrate, stretch, twist or bend out of plane, which is a less energetic state than exciting electrons to a higher energy level, while still increasing their potential energy. This allows for a slight increase the reactivity of the alkene to react with ozone since we are adding in energy into the system, and making the alkene more reactive, while avoiding possible collateral reactions on the molecule that the excitation to a higher energy level could induce. Compounds that have more than one light-absorbing moiety reacting to different wavelengths can add another level of control by, e.g., releasing different fragrance molecules with different wavelength stimulation by different LEDs.

Compounds that conduct electricity, e.g., conductive polymers (see, e.g., Ates et al., 2012), are also well-known. By combining those compounds with moieties comprising a reactive double bond, compounds are created that will be more reactive with ROS when exposed to an electric charge.

In these embodiments, compounds can be designed that react to a higher or lower voltage, and have electrical conduction to one or multiple reactive alkenes, that, upon reaction with a free radical, ROS or another reactive species, will release different compounds. The voltage can be generated from an AC or DC source, e.g., from a battery or from an external electrical source, generated by hydroelectric, solar or wind power, or fossil fuels. By adjusting the external voltage, current, and other factors, we can increase or decrease the reactivity of the free radical-, ROS— or another reactive species-reactive alkene by increasing or decreasing the amount of time a negative formal charge goes on the ozone reactive alkene.

As with other compounds discussed herein, the flow of electricity through the compounds can be adjusted by the addition of side chains that enhance or resist the flow of electricity through the compound.

The present invention is not limited to the use of any particular light intensity, electrical voltage, or light or electrical duration.

These light- and electricity-activating compounds are useful for multiple applications. They can be used in coatings, for example paints, or air filter coatings, where light or electricity flowing onto the coating can be controlled to activate, deactivate, or have decreased or increased activation of the free radical-, ROS— or another reactive species-degrading activity. The activation or deactivation (turning on or off the light or electricity) can be controlled to occur at any time desired, e.g., on a sunny day when ozone levels are high, or when an air filter is actively filtering air.

By using penetrating light wavelengths, the compounds can be activated through a physical surface that the light can penetrate.

Through this process, the compounds can protect a surface from UV and other wavelengths from damage by absorbing the energy, and putting the input of energy into the chemical reaction with the free radical, ROS or another reactive species, for more than one benefit. This application is desireable in skin care products, where sun protection can be provided by absorbing the energy from the sun at any choice of wavelengths depending on the absorption range or ranges of our conjugated systems, and transferring that energy to the free radical, ROS or another reactive species reactive alkene, protecting the skin from oxidation even further, while creating molecules that can also provide a benefit.

Since some compounds fluoresce or change color when activated, the compounds can be configured to provide a visual display when activated or when not activated.

The electricity-activating compounds can be deposited on a non-conductive or a conductive surface, to modify the flow of electrons. Additionally, other conductor polymers can be added to the system to help the flow of electrons, even if they don't remove ozone. Aromatics, lone pairs, and other pi electrons can be conjugated in different ways to increase or decrease the flow of electrons.

A particularly useful application is in air filters. Various compounds can be utilized that are activated by various wavelengths or voltages, where they can be differentially activated depending on the level or the free radical, ROS (e.g., ozone) or another reactive species in the environment. Additionally, an air filter in a car can be configured to turn on when a car starts, so it is activated when it is needed the most, or has a program that monitors your location, and adjusts filter capabilities automatically, or varies by speed, or whether traffic is stop and go.

Compounds CXXXVIII and CXXXIX are examples of compounds that are activated by both light and electricity. The polyaniline polymeric backbone is conductive, and the high degree of conjugation allows it to absorb light and excite an electron into the conduction band, and into a higher energy orbital, giving the free radical-, ROS— or another reactive species-reactive double bond greater electron density, even though it does not have a negative charge or extra electron. Thus, exposure of these compounds to light adds energy to the specific double bond by making one of the electrons in the system more energetic than before. Additionally, the electron from either light or electricity does not cause untoward reactions of free radical, ROS or another reactive species with the aromatic moieties since aromatics are too stable. Thus, only the alkene increases reactivity with free radicals, ROSs and other reactive species.

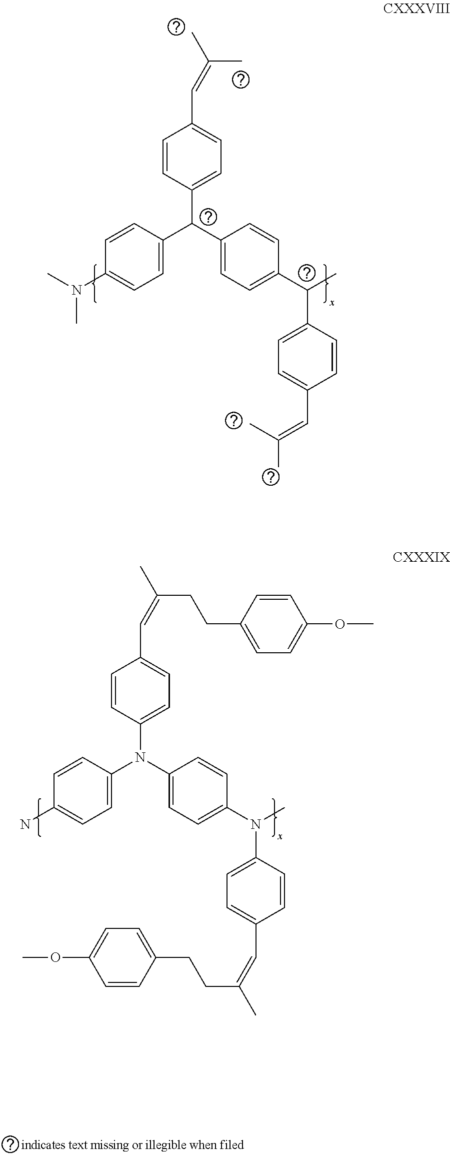

wherein X is an integer of between 1 and 1,000,000; and R₁ and R₂ are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

These polymers, on reaction with a free radical, an ROS (including ozone) or another reactive species, reactively eliminate the free radical, ROS or another reactive species, leaving the byproduct

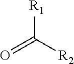

### for CXXXVIII and

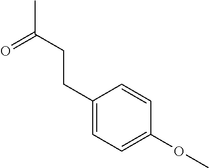

(a fragrance aromatic) for CXXXIX.

The alkenes in these compounds are very reactive as a result of having the very highly electron donating amine groups conjugated with it in para positions. These tertiary amines are highly activating electron donating groups because they are less electronegative than oxygen atoms, so they accept positive charges easier than oxygen atoms. They are also reactive because they accept electron density from their neighboring amines in a para position, essentially causing a cascade of resonance electron donating. Tertiary amines are also very stable, avoiding reaction with any moieties other than the alkene. Also, while compounds where the double bond reacts to form an aldehyde are typically more reactive than compounds that react to form a ketone, the ketone-producing compound CXXXIX is just as reactive as the aldehyde because the methyl group is close enough to the aromatic ring to cause steric hindrance, imparting some instability to the double bond, bringing the double bond to a slightly higher energy level as a result. It is important to note that the fragrance aromatic is not conjugated with the free radical-, ROS— and another reactive species-reactive alkene because that would give too much stabilization to it, and decrease the reactivity of our target alkene.

The above considerations for activation by light or electricity, and the effect of the various structural components on activation can be utilized in the design of other free radical-, ROS— and another reactive species-reactive compounds, as appropriate.

The copolymer compounds CXL and CXLI utilize a Y group that breaks up the conjugation of the polymer backbone chain altering, through the use of different ratios of the monomers, the absorbance of wavelengths of light for specific ranges. This is because the longer the conjugation chain, the lower energy wavelengths it will absorb. Spacer groups such as the Y groups here can also be utilized in the design of other free radical-, ROS— and another reactive species-reactive compounds.

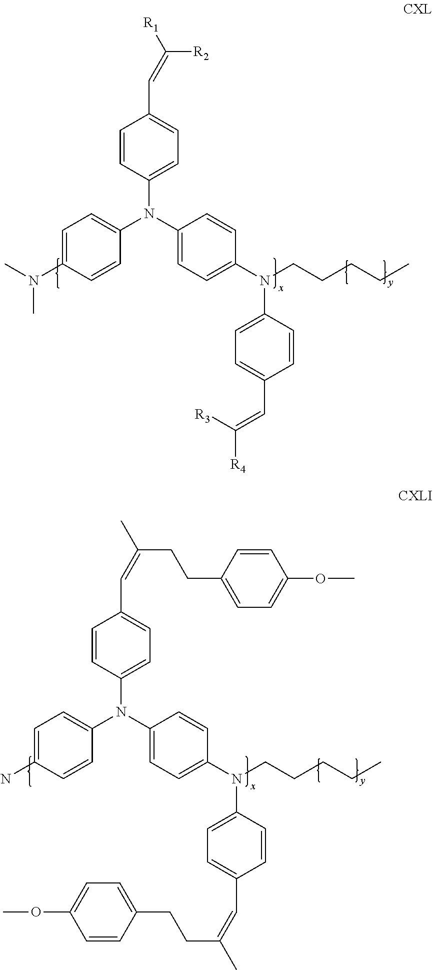

wherein X and Y are independently integers of between 1 and 1,000,000; and R₁, R₂, R₃ and R₄ are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

Compounds CXLII and CXLIII are examples of copolymers that have two different absorption spectrums, allowing the targeting of one or the other monomer with specific wavelengths. When such copolymers are designed with different R groups (e.g., R₁ and R₂ being different from R₅ and R₆ in compound CXLII), different products are produced depending on whether the activating light activates one or the other monomer. It is noted that monomer Y does not have a conjugated backbone chain, while monomer X does.

This use of copolymers (including with any number of different monomers) having monomers activated by different wavelengths, or one activated by light and another by electricity, or by different voltages of electricity, or different intensities of light, is contemplated for the design of free radical-, ROS— and another reactive species-reactive compounds.

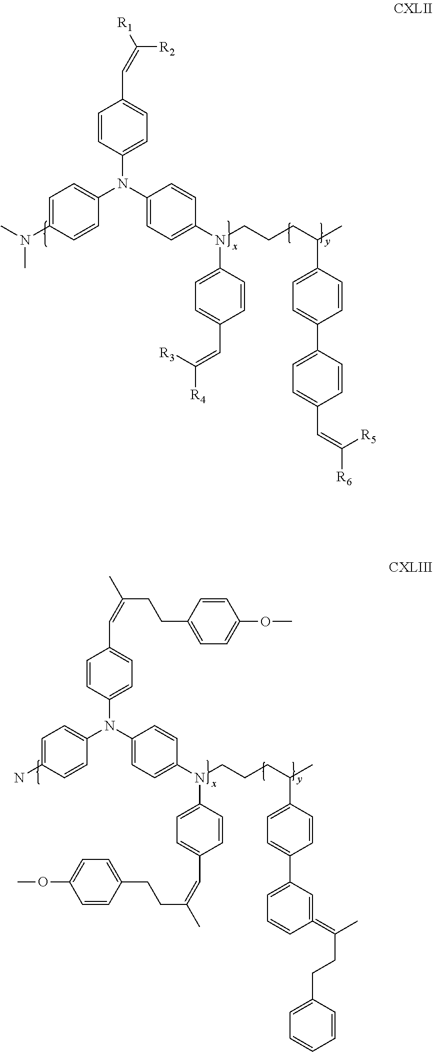

wherein X and Y are independently integers of between 1 and 1,000,000; and R₁, R₂, R₃, R₄, R₅ and R₆, are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

Compound CXLIV is an example of the use of a fluorescent dye to create an free radical-, ROS— and another reactive species-reactive compound. Upon activation with light having a wavelength of 384 nm, this compound reacts with a free radical, ROS or another reactive species to form 2,4-Diphenyl-2-methoxy-3(2H)-furanone, having an excitation wavelength at 384 nm and an emission wavelength at 472 nm in acetonitrile.

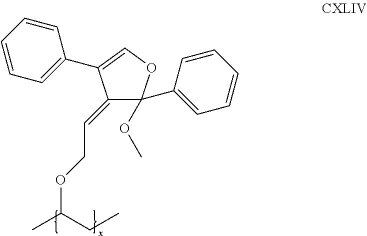

wherein X is an integer of between 1 and 1,000,000.

Compound CXLV produces naphthalene-2,3-dicarboxaldehyde upon reaction with a free radical, an ROS— or another reactive species. Commonly used in biological research, that compound is essentially nonfluorescent until reacted with a primary amine in the presence of excess cyanide or a thiol, to yield a fluorescent isoindole with excitation/emission maxima −419/493 nm.

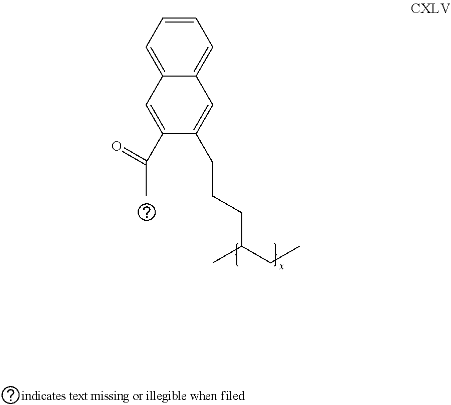

wherein X is an integer of between 1 and 1,000,000.

Compounds CXLVI, CXLVII, CXLVIII, CXLIX, CL, and CLI are activated with infrared light. These compounds are stronger electron donators than oxygen donators since the tertiary amine has less electronegativity and increased electron density and stability. The use of similar compounds with secondary amines is also contemplated, as is copolymers with different monomers than in compounds CL and CLI.

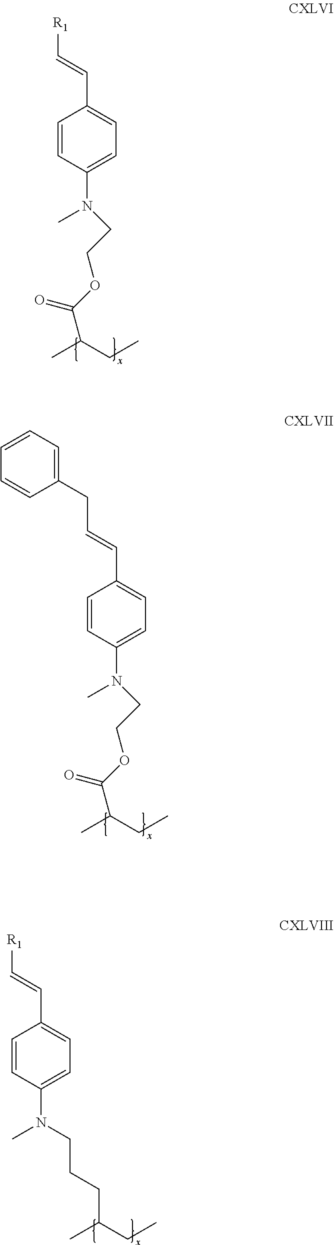

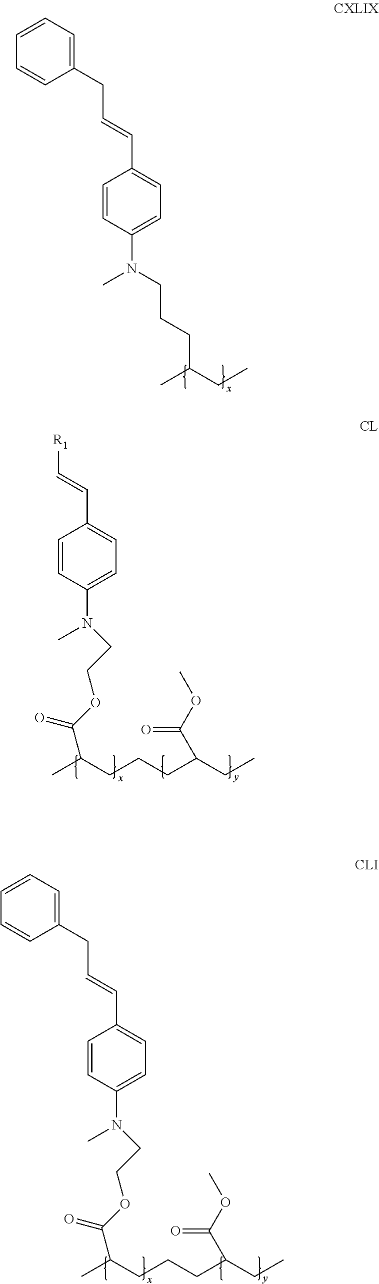

wherein X and Y are independently integers of between 1 and 1,000,000; R₁ is a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

It is to be understood that each compound provided in this specification can be further generalized, e.g., by replacing the specific moiety distal to the reactive alkene or alkyne with an R group, where any R is independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl. Additionally, any polymer provided in this specification can be provided as a monomer or as a polymer or copolymer with any other polymeric or copolymeric backbone provided herein or in PCT Publications WO 2016/023015 or WO 2017/049305, and any monomer can be provided as a polymer or copolymer, with any of the polymeric or copolymeric backbones provided herein or in PCT Publications WO 2016/023015 or WO 2017/049305. With any of the polymers and copolymers provided herein, there may be some polymer units that do not have the activating group attached.

The polymers described herein are not narrowly limited to any particular number of monomeric units, and can generally be produced in any monomer number up to 1,000,000 or more, for example 10, 25, 50, 100, 200, 500, 1000, 2000, 5000, 10,000, 20,000, 50,000, 100,000, 200,000, 500,000 or any number in between or greater or less.

Any of the compounds provided herein can also be covalently or noncovalently bound to, or incorporated in, any degradable (e.g., biodegradable) or nondegradable material by any method known in the art, for example a glass, a metal, a biological or an organic surface (e.g., activated carbon) or membrane (e.g., micelles) or in internal areas such as openings or microencapsulations thereof, including particles of any size (e.g., a nanoparticle or microparticle), used for any purpose. Nonlimiting examples include a paint, a spray, a solid material, coated on the surface of a solid material, an item of clothing, a fan, rotating blades for more air exposure, a pharmaceutical, a skin care product, a fabric, a carpet, a paint, a sealant, a finish, an air filter, a water filter, a face-mask, a cosmetic, a cream, a lotion, a wipe, a cloth, a coating, a cleaner, an air freshener, a window cleaner, a food, an animal feed, a bag or product packaging.

### Synthesis

Also provided herewith are methods of synthesizing compound CCC, which includes alkenes provided herein that react with free radicals, ROS, or another reactive species.

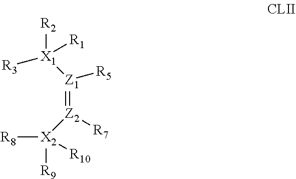

wherein

with respect to X₁ and X₂, CLII can be cis or trans, or a mixture thereof;

X₁ and X₂ are each independently O, P, N, C, Si or S;

Z₁ and Z₂ are each independently C, S, P, N or Si; and

R₁, R₂, R₃, R₄, R₅, R₆, R₇, R₈, R₉, and R₁₀ are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl. The method comprises

combine compound CLIII with CLIV in the presence of a first deprotonating agent or a base

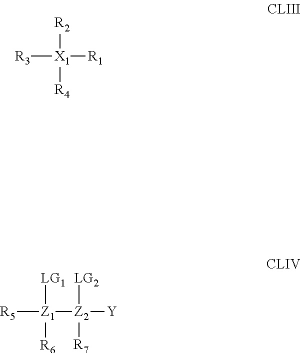

wherein

Y is a halogen, and

LG₁ and LG₂ are each independently a leaving group selected from the group consisting of a halogen, a tosylate, a mesylate, water, an alcohol, dinitrogen, a dialkyl ether, a perfluoroalkylsulfonate, a nitrate, a phosphate, an inorganic ester, an ester, a thioether, an amine, ammonia, a carboxylate, an aromatic, a substituted amine, an amide, an alkoxide, and a hydroxide,

to form Mixture 1;

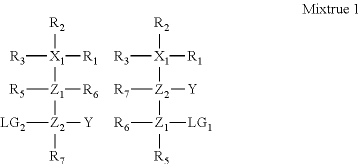

combine Mixture 1 with compound CLV in the presence of a second deprotonating agent or base to form Mixture 2;

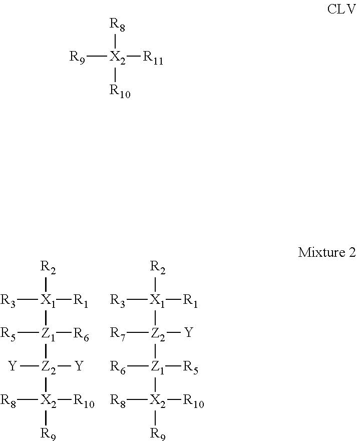

wherein R₁₁ is a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl,

and react Mixture 2 with a third deprotonating agent or base to form CCC.

Any deprotonating agent known in the art can be used in this method. Particularly useful deprotonating agents are NaH or KH. The skilled artisan can determine useful deprotonating agents for synthesizing any particular compound using this scheme without undue experimentation. Similarly, the skilled artisan can determine useful leaving groups to use in the synthesis of any particular compound using this scheme without undue experimentation.

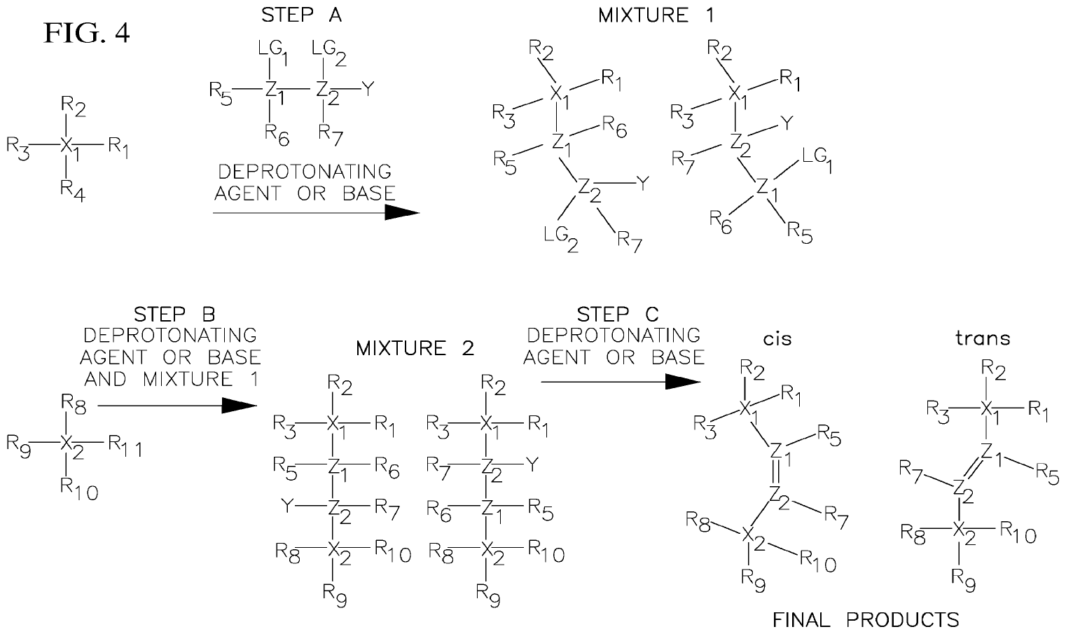

This method is illustrated in FIG. 4. The final product illustrated therein is a mixture of the cis and trans isomers. If desired, the isomers

In some embodiments, CLII is polymerized. See, e.g., compounds CLVIII, CLXII, and CLXIV below. In other embodiments, CLII is not polymerized.

In various embodiments, Z₁═Z₂ and/or R₅═R₇. In additional embodiments, R₆ is H and/or R₄ and/or R₁₁ are each independently either a lone pair of electrons or H.

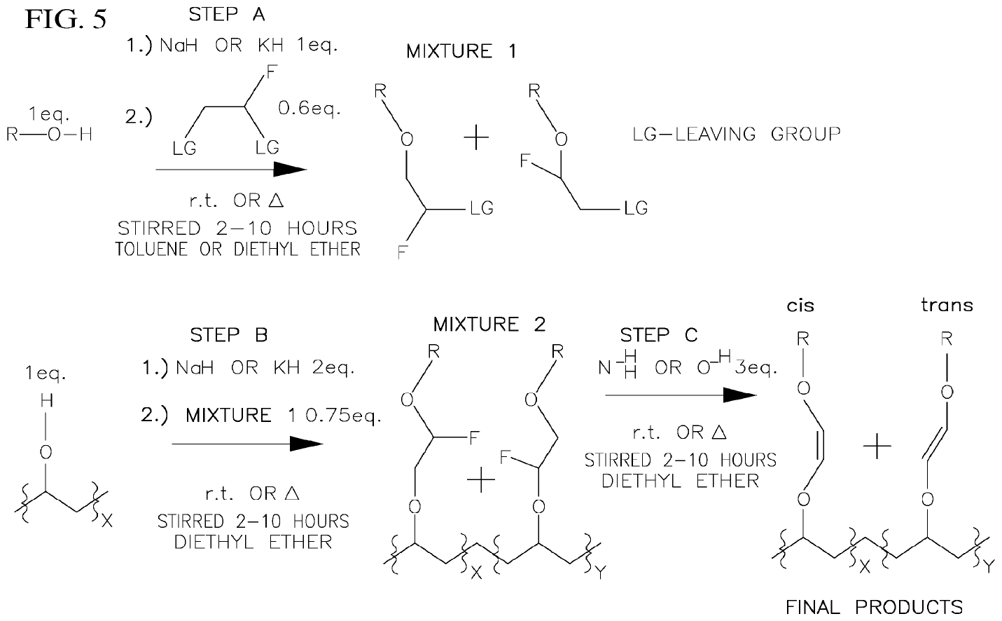

A example of this synthesis scheme is illustrated in FIG. 5. In those embodiments, the first and second deprotonating agent is NaH or KH; the third deprotonating agent is NH₂⁻ or OH⁻; CLIII is R₆—H; CLIV is CLVI; Mixture 1 is Mixture 3; CLV is CLVII; Mixture 2 comprises Mixture 4; and the final product is CLVIII, as provided below.

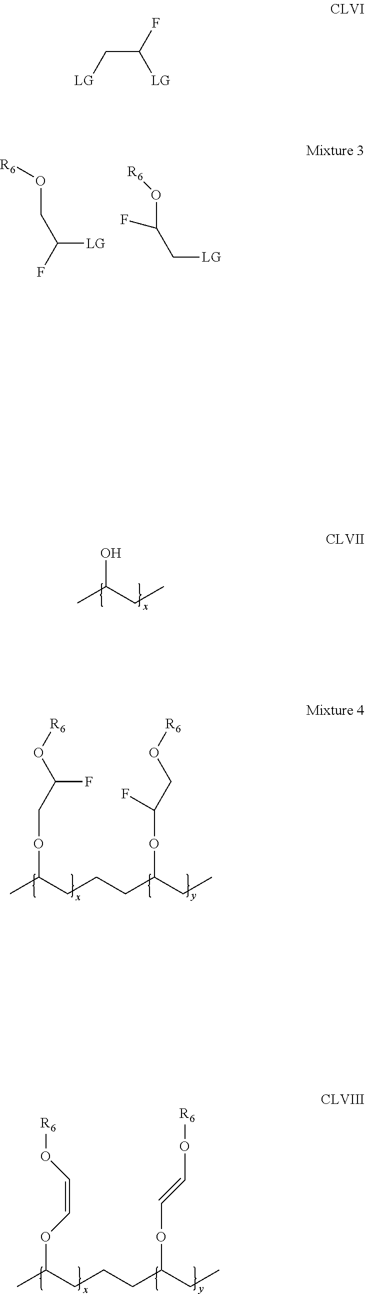

wherein X and Y are each independently an integer from 1-1,000,000.

In some of those embodiments, the first and second deprotonating agent is NaH or KH; the third deprotonating agent is NH₂⁻ or OH⁻; CLIII is CLIX; CLIV is CLX; Mixture 1 is Mixture 5; CLV is CLXI; and Mixture 2 comprises Mixture 6, as illustrated below.

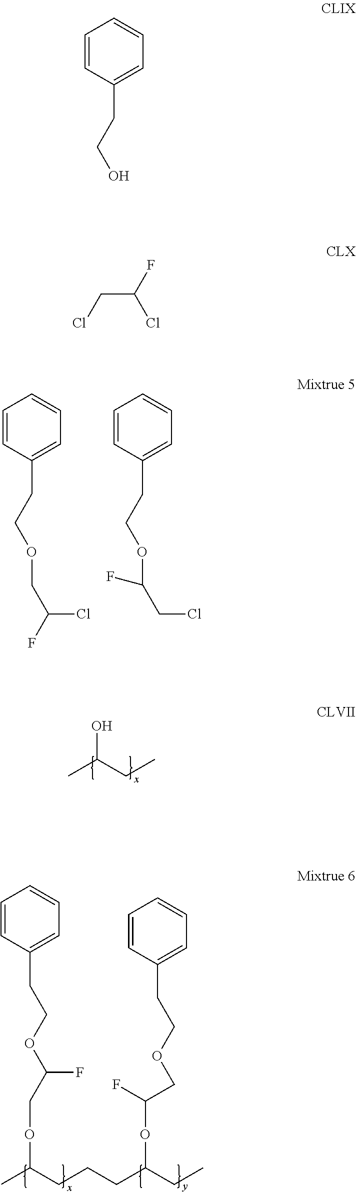

wherein X and Y are each independently an integer from 1-1,000,000.

In some of these copolymer embodiments, a third copolymer component is incorporated into the compound. The third copolymer component is provided in these examples where the ratio of the polymer, here exemplified as CLVII, to Mixture 1 (e.g., Mixture 5) is high, so that each polymer “attachment point” does not have a Mixture 1 moiety. This leaves the “empty” polymer attachment point available for covalent binding of the third copolymer component, or allows it to remain the same. Under those circumstances, in the above example, Mixture 4 is Mixture 7.

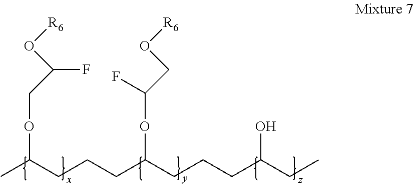

wherein Z is an integer from 1-1,000,000.

Here, for example, Cl—R₁₂ can added with Mixture 7 and the third deprotonating agent, providing the final product CLXII.

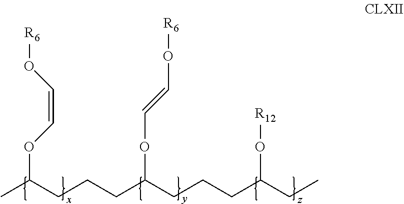

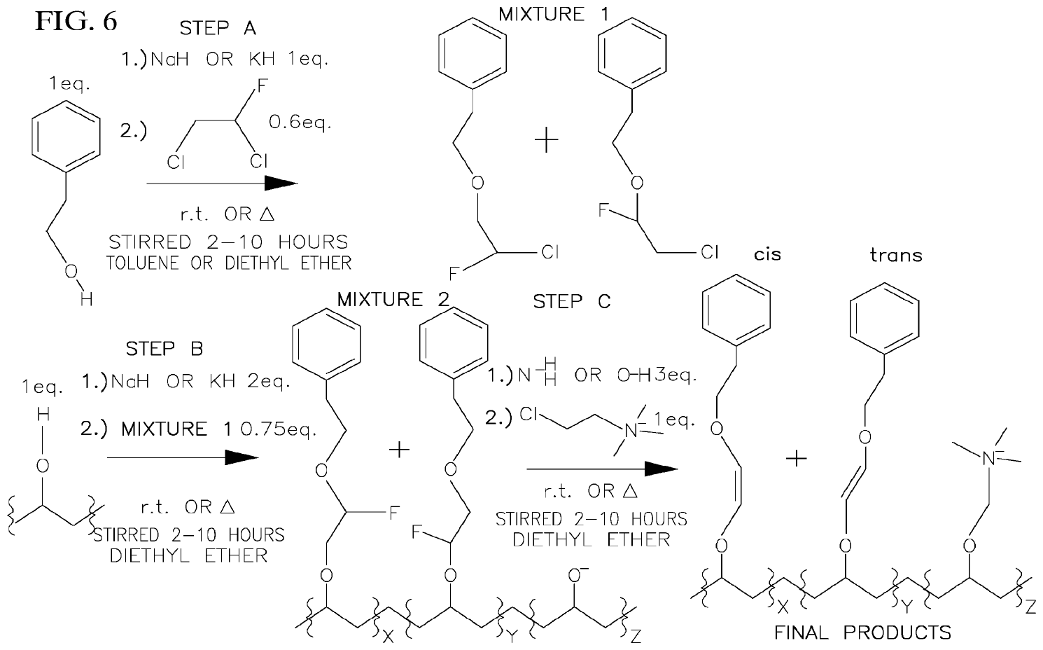

In a specific embodiment, illustrated in FIG. 6, compound CLXIII is added with Mixture 6 and the third deprotonating agent, and the final product is CLXIV.

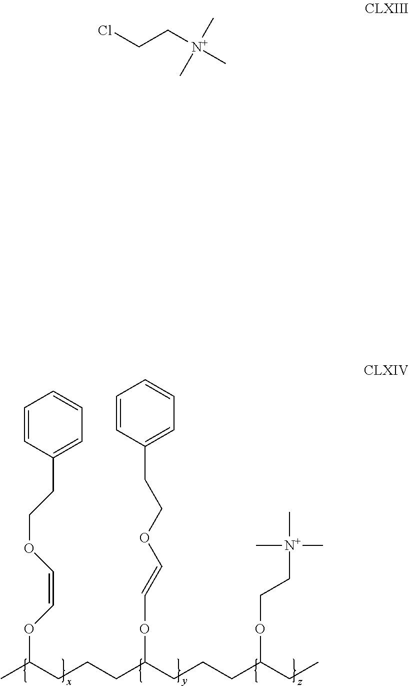

wherein Z is an integer from 1-1,000,000.

For any step of any of these synthesis schemes, the skilled artisan could establish effective reaction conditions without undue experimentation, since all of the individual steps are established and predictable.

### REFERENCES

Arulselvan et al., 2016, Oxidative Med. Cell. Longevity 2016:5276130.
Ates et al., 2012, Current Physical Chemistry 2:224-240.
Braverman and Moser, 2012, Biochim. Biophys. Acta 1822:1442-1452.
Finosh and Jayabalan, 2013, Adv. Biosci. Biotechnol. 4:1134-1146.
Forte et al., 2016, Oxidative Med. Cell. Longevity 2016:7364138.
Hasson et al., 2001, J. Geophysical Res. 106:34131-34142.
Kalinowski et al., 2014, J. Phys. Chem. A 119:2318-2325.
Kelly, 2003, Occup. Environ. Med. 60:612-616.
Lalkovicova and Danielisova, 2016, Neural Regen. Res. 11:865-874.
Liu et al., 2014, Science 345:1596-1598.
Long et al., 2016, J. Am. Chem. Soc. 138:14409-14422.
Nguyen et al., 2006, Phys. Chem. Chem. Phys. 18:10241-10254.
Panth et al., 2016, Adv. Med. 2016:9152732.
Pryor et al., 1982, J. Am. Chem. Soc. 104:6685-6692.
Sindelar et al., 1999, Free Radical Biol. Med. 26:318-324.
Sinha, 2013, Clin. Toxicol. S13:e001.
Soufli et al., 2016, World J. Gastrointest. Pharmacol. Ther. 7:353-360.
Stadelmann-Ingrand et al., 2001, Free Radical Biol. Med. 31:1263-1271.
Su et al., 2014, Nature Chem. 6:477-483.
Taatjes, 2017, Annu. Rev. Phys. Chem. 68:183-207.
Thukkani et al., 2003, Circulation 108:3128-3133.
Ullah et al., 2015, Saudi Pharma. J. 24:547-553.
Wilkinson, Sandra. “Alkynes.” SlidePlayer, 2015, slideplayer.com/slide/6246065/.
Womack et al., 2015, Sci. Adv. 2015; 1:e1400105.
Wynalda and Murphy, 2010, Chem. Res. Toxicol. 23:108-117.
http://crab.rutgers.edu/˜alroche/Ch17.pdf.
PCT Publication No. WO 2000/053722.
PCT Patent Publication WO 2016/023015.
PCT Patent Publication WO 2017/049305.
U.S. Provisional Patent Application 62/428,137.
U.S. Provisional Patent Application 62/542,404.
U.S. Pat. No. 2,773,072.
US Patent Application Publication 2003/0026841.
U.S. Patent Application Publication 2008/0076701.
European Patent EPI 1102785B1.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A compound that generates a peroxide when it reacts with ozone in the presence of water, the compound comprising the structure VIII, IX, or X 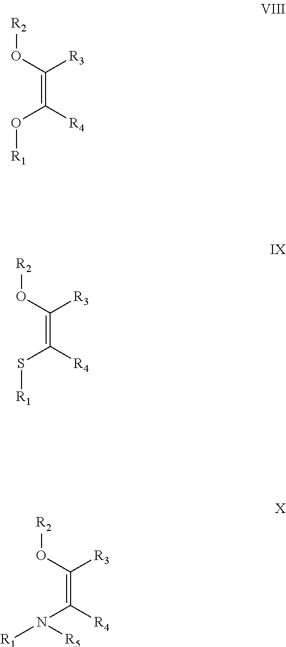
wherein R₁, R₂, R₃, R₄ and R₅ are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, a conjugated or unconjugated group, an electron donating and/or electron withdrawing conjugated and/or unconjugated group, a halogen, a substituted or unsubstituted arylalkyl, or a substituted or unsubstituted heteroarylalkyl.

2. The compound of claim 1, wherein the peroxide is hydrogen peroxide, an organic peroxide, an organic hydroperoxide, a peracid, a peroxide ion, superoxide, benzoyl peroxide, performic acid, peracetic acid, meta-chloroperoxybenzoid acid, peroxybenzoic acid, a peroxy acid, or R—O—O—R.

3. The compound of claim 1, wherein the compound is a monomer.

4. The compound of claim 3, wherein the monomer is less than 1000 MW.

5. The compound of claim 1, wherein the compound is a polymer.

6-9. (canceled)

10. The compound of claim 1, comprising the structure XI, XII, XIII, or XIV 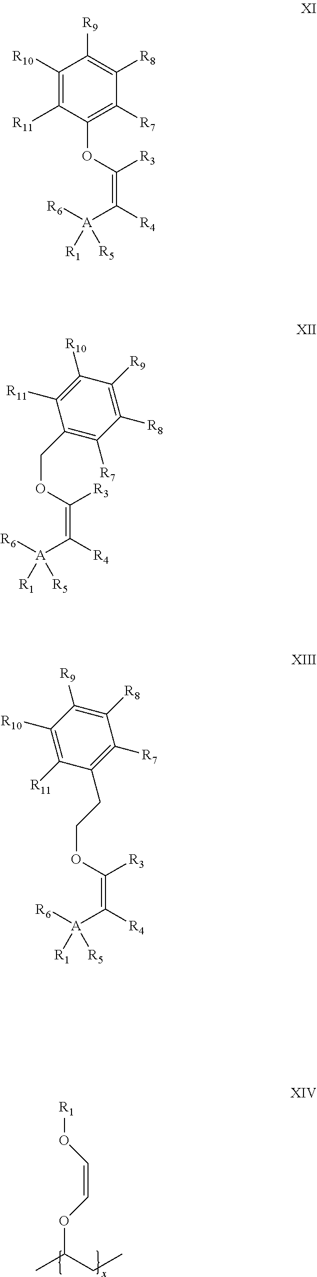
wherein A is O, N, S or P, X is an integer from 1 to 1,000,000, and R₁, R₃, R₄, R₅, R₆, R₇, R₈, R₉, R₁₀, and R₁₁ are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group, conjugated or unconjugated groups, electron donating and/or electron withdrawing conjugated and/or unconjugated groups, a halogen, substituted or unsubstituted arylalkyl, or substituted or unsubstituted heteroarylalkyl.

11. (canceled)

12. The compound of claim 10, comprising the structure XV, XVI, XVII, XVIII, XIX, XX, XXII, XXIII, XLIII, XLIV, XXXIX, XXXV, LIII, or XXI 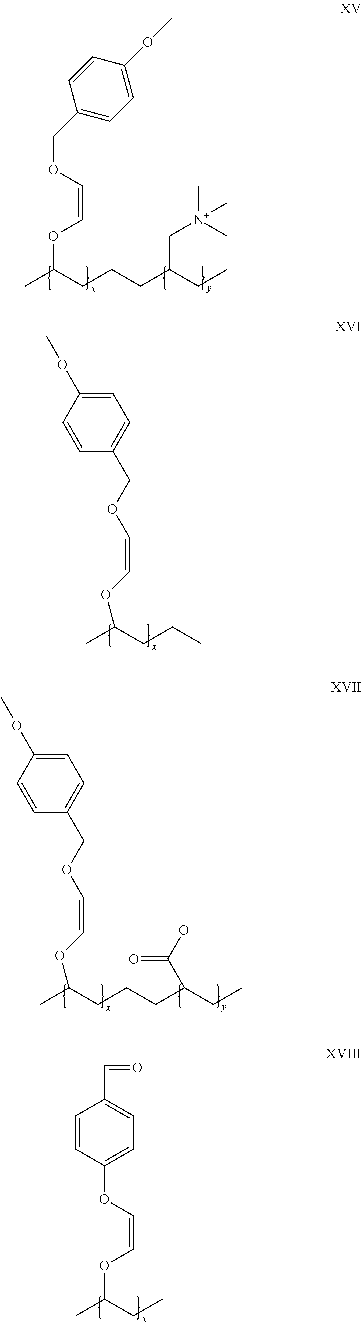 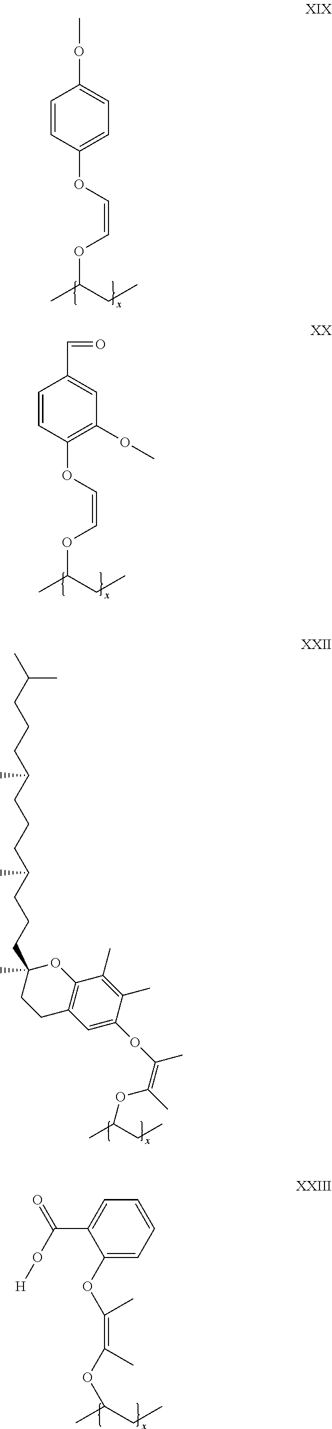 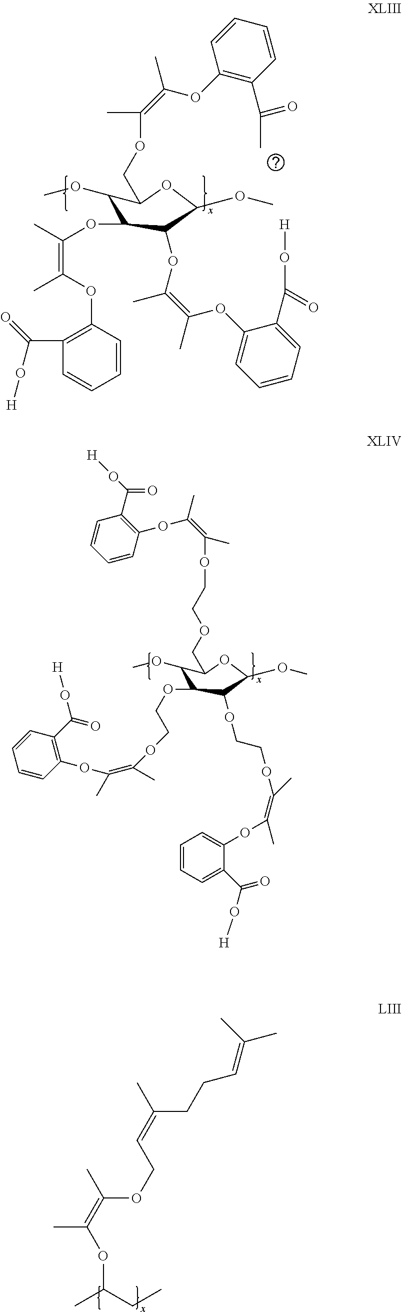 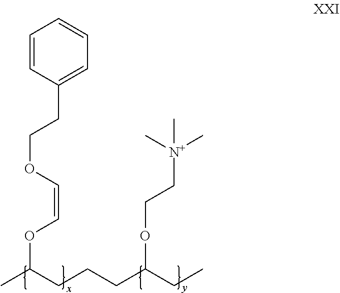
wherein X and Y are independently integers from 1 to 1,000,000.

13. (canceled)

14. The compound of claim 1, wherein the compound is formulated as a skin treatment composition or as a coating.

15. (canceled)

16. A method of generating a peroxide, the method comprising reacting the compound of claim 1 with ozone in the presence of water.

17. The method of claim 16, wherein the compound is formulated as a skin treatment composition or as a coating.

18-87. (canceled)

88. The compound of claim 1, which is inactive and releases an active or useful molecule when the compound reacts with ozone.

89. The compound claim 88, wherein the active or useful molecule is a food ingredient, a specific binding agent, a biocide, a fragrance, a cosmetic, a dye, an antioxidant, a fertilizer, a nutrient, or a pharmaceutical.

90. (canceled)

91. The compound of claim 89, wherein active or useful molecule is a food ingredient, wherein the food ingredient is a sugar, vanillin, or veratraldehyde.

92. The compound of claim 89, wherein the active agent is a biocide.

93-94. (canceled)

95. The compound of claim 92, wherein the biocide is an antimicrobial.

96-98. (canceled)

99. A method of decomposing a free radical, a reactive oxygen species (ROS) or another reactive species, the method comprising contacting the free radical, the ROS, or the another reactive species with the compound of claim 1.

100. The method of claim 100, wherein the free radical, the ROS, or the another reactive species is ozone.

101-104. (canceled)

105. The method of claim 99, wherein the compound is incorporated into a paint, a spray, a solid material, coated on the surface of a solid material, an item of clothing, a fabric, a carpet, a paint, a sealant, a finish, an air filter, a water filter, a face-mask, a cosmetic, a cream, a pharmaceutical, a lotion, a wipe, a cloth, a coating, a cleaner, an air freshener, a window cleaner, a food, an animal feed, a bag or product packaging.

106. A paint, a spray, a solid material, an item of clothing, a fabric, a carpet, a paint, a sealant, a finish, an air filter, a water filter, a face-mask, a cosmetic, a cream, a lotion, a wipe, a cloth, a coating, a cleaner, an air freshener, a window cleaner, a pharmaceutical, a food, an animal feed, a bag or product packaging comprising the compound of claim 1.

107-116. (canceled)
